(19) United States
(12) Patent Application Publication
(10) Pub. No.: US 2025/0145592 A1
(43) Pub. Date: May 8, 2025

(54) COMPOUNDS, COMPOSITIONS, AND METHODS

(71) Applicant: NICO Therapeutics, Inc., Chicago, IL (US)

(72) Inventors: Alex L. Bagdasarian, South San Francisco, CA (US); Robert A. Craig, II, South San Francisco, CA (US); Javier de Vicente Fidalgo, Foster City, CA (US); Anthony A. Estrada, Danville, CA (US); Brian M. Fox, Redwood City, CA (US); Cheng Hu, San Mateo, CA (US); Benjamin J. Huffman, Brisbane, CA (US); Katrina W. Lexa, San Francisco, CA (US); Lizanne G. Nilewski, Pacifica, CA (US); Maksim Osipov, Redwood City, CA (US); Arun Thottumkara, San Francisco, CA (US)

(21) Appl. No.: 18/837,817

(22) PCT Filed: Feb. 15, 2023
(86) PCT No.: PCT/US2023/013164

§ 371 Date: Aug. 12, 2024

Related U.S. Application Data
(60) Provisional application No. 63/311,011, filed on Feb. 16, 2022.

(51) Int. Cl. C07D 403/12; A61K 31/502; A61K 31/506; A61K 31/53; C07D 237/32; C07D 471/04

(52) U.S. Cl. CPC C07D 403/12; A61K 31/502; A61K 31/506; A61K 31/53; C07D 237/32; C07D 471/04

(57) ABSTRACT

The present disclosure relates generally to small molecule modulators of NLR Family Pyrin Domain Containing 3 (NL-RP3), or a pharmaceutically acceptable salt, isotopically enriched analog, stereoisomer, mixture of stereoisomers, or prodrug thereof, methods of making and intermediates thereof, and methods of using thereof. Further disclosed a method for treating a disease or condition mediated, at least in part, by NLRP3, the method comprising administering an effective amount of the pharmaceutical composition, to a subject in need thereof.

### CROSS REFERENCE TO RELATED APPLICATIONS

This application claims the benefit under 35 U.S.C. § 119(e) to U.S. Provisional Application No. 63/311,011, filed Feb. 16, 2022, which is incorporated by reference in its entirety.

### FIELD

The present disclosure relates generally to small molecule modulators of NLR Family Pyrin Domain Containing 3 (NLRP3), and their use as therapeutic agents.

### BACKGROUND

Inhibition of NLRP3 activation has been shown to result in potent therapeutic effects in animal models of inflammatory diseases. Modulators of NLRP3, inhibitors in particular, have broad therapeutic potential in a wide array of auto-inflammatory and chronic inflammatory diseases that either require better treatment options or for which no adequate therapies exist. Therapies targeting NLRP3-dependent cytokines are already approved for therapeutic use; however, they have notable disadvantages relative to direct NLRP3 antagonists. There remains a strong impetus for the discovery and clinical development of molecules that antagonize NLRP3.

### DESCRIPTION

Provided herein are compounds, or a pharmaceutically acceptable salt, isotopically enriched analog, stereoisomer, mixture of stereoisomers, or prodrug thereof, that are useful in treating and/or preventing diseases mediated, at least in part, by NLRP3.

In some embodiments, provided are compounds that modulate the activity of NLRP3. In some embodiments, the compounds inhibit the activation of NLRP3.

In another embodiment, provided is a pharmaceutical composition comprising a compound as described herein, or a pharmaceutically acceptable salt, isotopically enriched analog, stereoisomer, mixture of stereoisomers, or prodrug thereof, and a pharmaceutically acceptable carrier.

In another embodiment, provided is a method for treating a disease or condition mediated, at least in part, by NLRP3, the method comprising administering an effective amount of the pharmaceutical composition comprising a compound as described herein, or a pharmaceutically acceptable salt, isotopically enriched analog, stereoisomer, mixture of stereoisomers, or prodrug thereof.

In another embodiment, provided is a method for treating a disease or condition mediated, at least in part, by TNF-α, the method comprising administering an effective amount of the pharmaceutical composition comprising a compound as described herein, or a pharmaceutically acceptable salt, isotopically enriched analog, stereoisomer, mixture of stereoisomers, or prodrug thereof. In some embodiments the administration is to a subject resistant to treatment with an anti-TNF-α agent. In some embodiments, the disease is a gut disease or condition. In some embodiments, the disease or condition is inflammatory bowel disease, Crohn's disease, or ulcerative colitis.

The disclosure also provides compositions, including pharmaceutical compositions, kits that include the compounds, or a pharmaceutically acceptable salt, isotopically enriched analog, stereoisomer, mixture of stereoisomers, or prodrug thereof, methods of using (or administering) and making the compounds, or a pharmaceutically acceptable salt, isotopically enriched analog, stereoisomer, mixture of stereoisomers, or prodrug thereof, and intermediates thereof.

The disclosure further provides compounds, or a pharmaceutically acceptable salt, isotopically enriched analog, stereoisomer, mixture of stereoisomers, or prodrug thereof, or compositions thereof for use in a method of treating a disease, disorder, or condition that is mediated, at least in part, by NLRP3.

Moreover, the disclosure provides uses of the compounds, or a pharmaceutically acceptable salt, isotopically enriched analog, stereoisomer, mixture of stereoisomers, or prodrug thereof, or compositions thereof in the manufacture of a medicament for the treatment of a disease, disorder, or condition that is mediated, at least in part, by NLRP3.

The description herein sets forth exemplary embodiments of the present technology. It should be recognized, however, that such description is not intended as a limitation on the scope of the present disclosure but is instead provided as a description of exemplary embodiments.

### 1. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through a line in a structure indicates a specified point of attachment of a group. Unless chemically or structurally required, no directionality or stereochemistry is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

Provided are also a pharmaceutically acceptable salt, isotopically enriched analog, deuterated analog, stereoisomer, mixture of stereoisomers, and prodrugs of the compounds described herein. “Pharmaceutically acceptable” or “physiologically acceptable” refer to compounds, salts, compositions, dosage forms, and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, aluminum, ammonium, calcium, and magnesium salts. Salts derived from organic bases include, but are not limited to, salts of NH₃, or primary, secondary, tertiary amines, such as salts derived from a N-containing heterocycle, a N-containing heteroaryl, or derived from an amine of formula N(R^{N})₃ (e.g., HN⁺(R^{N})₃ or (alkyl)N⁺(R^{N})₃) where each R^{N} is independently hydrogen, alkyl, alkenyl, alkynyl, haloalkyl, cycloalkyl, heterocyclyl, aryl, or heteroaryl, wherein each is optionally substituted, such as by one or more (e.g., 1-5 or 1-3) substituents (e.g., halo, cyano, hydroxy, amino, alkyl, alkenyl, alkynyl, haloalkyl, alkoxy, or haloalkoxy). Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

“Alkyl” refers to an unbranched or branched saturated hydrocarbon chain. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl) or 1 to 4 carbon atoms (i.e., C₁₋₄ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂), and tert-butyl (i.e., —C(CH₃)₃); and “propyl” includes n-propyl (i.e., —(CH₂)₂CH₃) and isopropyl (i.e., —CH(CH₃)₂).

“Alkenyl” refers to an alkyl group containing at least one (e.g., 1-3, or 1) carbon-carbon double bond and having from 2 to 20 carbon atoms (i.e., C₂₋₂₀ alkenyl), 2 to 12 carbon atoms (i.e., C₂₋₁₂ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include, e.g., ethenyl, propenyl, butadienyl (including 1,2-butadienyl and 1,3-butadienyl).

“Alkynyl” refers to an alkyl group containing at least one (e.g., 1-3, or 1) carbon-carbon triple bond and having from 2 to 20 carbon atoms (i.e., C₂₋₂₀ alkynyl), 2 to 12 carbon atoms (i.e., C₂₋₁₂ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy, and 1,2-dimethylbutoxy.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl, or heteroaryl.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl, and anthryl. Aryl, however, does not encompass or overlap in any way with heteroaryl defined below. If one or more aryl groups are fused with a heteroaryl, the resulting ring system is heteroaryl regardless of point of attachment. If one or more aryl groups are fused with a heterocyclyl, the resulting ring system is heterocyclyl regardless of point of attachment. If one or more aryl groups are fused with a cycloalkyl, the resulting ring system is cycloalkyl regardless of point of attachment.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl), 3 to 14 ring carbon atoms (i.e., C₃₋₁₂ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Monocyclic groups include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl, and the like. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Halogen” or “halo” refers to atoms occupying group VIIA of the periodic table, such as fluoro, chloro, bromo, or iodo.

“Haloalkyl” refers to an unbranched or branched alkyl group as defined above, wherein one or more (e.g., 1 to 6 or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Haloalkoxy” refers to an alkoxy group as defined above, wherein one or more (e.g., 1 to 6 or 1 to 3) hydrogen atoms are replaced by a halogen.

“Heteroalkyl” refers to an alkyl group in which one or more of the carbon atoms (and any associated hydrogen atoms), excluding any terminal carbon atom(s), are each independently replaced with the same or different heteroatomic group, provided the point of attachment to the remainder of the molecule is through a carbon atom. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR^{y}—, —O—, —S—, —S(O)—, —S(O)₂—, and the like, wherein R is hydrogen, alkyl, alkenyl, alkynyl, cycloalkyl, heterocyclyl, aryl, heteroalkyl, or heteroaryl; each of which may be optionally substituted, as defined herein. Examples of heteroalkyl groups include, e.g., ethers (e.g., —CH₂OCH₃, —CH(CH₃)OCH₃, —CH₂CH₂OCH₃, —CH₂CH₂OCH₂CH₂OCH₃, etc.), thioethers (e.g., —CH₂SCH₃, —CH(CH₃)SCH₃, —CH₂CH₂SCH₃, —CH₂CH₂SCH₂CH₂SCH₃, etc.), sulfones (e.g., —CH₂S(O)₂CH₃, —CH(CH₃)S(O)₂CH₃, —CH₂CH₂S(O)₂CH₃, —CH₂CH₂S(O)₂CH₂CH₂OCH₃, etc.), and amines (e.g., —CH₂NR^{y}CH₃, —CH(CH₃)NR^{y}CH₃, —CH₂CH₂NR^{y}CH₃, —CH₂CH₂NR^{y}CH₂CH₂NR^{y}CH₃, etc., where R^{y} is hydrogen, alkyl, alkenyl, alkynyl, cycloalkyl, heterocyclyl, aryl, heteroalkyl, or heteroaryl; each of which may be optionally substituted, as defined herein). As used herein, heteroalkyl includes 2 to 10 carbon atoms, 2 to 8 carbon atoms, or 2 to 4 carbon atoms; and 1 to 3 heteroatoms, 1 to 2 heteroatoms, or 1 heteroatom.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen, and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen, and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or partially unsaturated cyclic alkyl group, with one or more ring heteroatoms independently selected from nitrogen, oxygen, and sulfur. The term “heterocyclyl” includes heterocycloalkenyl groups (i.e., the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro, and may comprise one or more (e.g., 1 to 3) oxo (═O) or N-oxide (—O—) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to a cycloalkyl, an aryl, or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋ₛ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur, or oxygen. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), thiomorpholinyl, thiamorpholinyl, 1-oxo-thiomorpholinyl, and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

The terms “optional” or “optionally” means that the subsequently described event or circumstance may or may not occur and that the description includes instances where said event or circumstance occurs and instances in which it does not. Also, the term “optionally substituted” refers to any one or more (e.g., 1 to 5 or 1 to 3) hydrogen atoms on the designated atom or group may or may not be replaced by a moiety other than hydrogen.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example, by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life, reduced dosage requirements, and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino, and/or carboxyl groups, or groups similar thereto.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds described herein, or their pharmaceutically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and/or fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers, or mixtures thereof, and includes “enantiomers,” which refers to two stereoisomers whose molecules are nonsuperimposeable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Prodrugs” means any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate, and benzoate derivatives), amides, guanidines, carbamates (e.g., N,N-dimethylaminocarbonyl) of hydroxy functional groups in compounds described herein, and the like. Preparation, selection, and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

### 2. Compounds

Provided herein are compounds that are modulators of NLRP3. In certain embodiments, provided is a compound selected from Table 1, or a pharmaceutically acceptable salt, isotopically enriched analog, prodrug, stereoisomer, or a mixture of stereoisomers thereof:

TABLE 1

| Ex. | Structure |
|---|---|
| 1 | 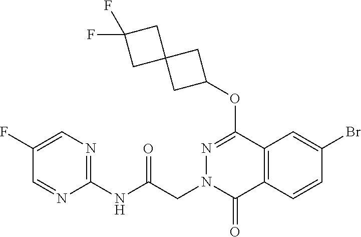 [SMILES: O=C(Cn1nc(OC2CC3(C2)CC(F)(F)C3)c2cc(Br)ccc2c1=O)Nc1ncc(F)cn1] |
| 2 | 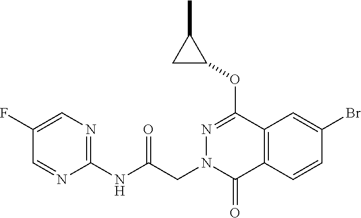 first eluting isomer |
| 3 | 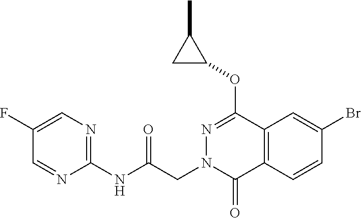 second eluting isomer |
| 4 | 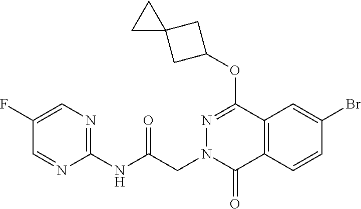 [SMILES: O=C(Cn1nc(OC2CC3(CC3)C2)c2cc(Br)ccc2c1=O)Nc1ncc(F)cn1] |
| 5 | 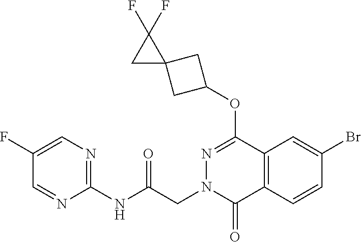 [SMILES: O=C(Cn1nc(OC2CC3(C2)CC3(F)F)c2cc(Br)ccc2c1=O)Nc1ncc(F)cn1] first eluting isomer |
| 6 | 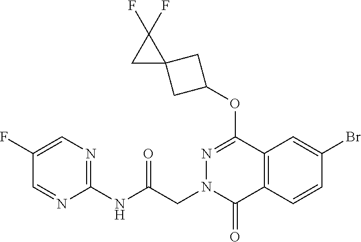 [SMILES: O=C(Cn1nc(OC2CC3(C2)CC3(F)F)c2cc(Br)ccc2c1=O)Nc1ncc(F)cn1] second eluting isomer |
| 7 | 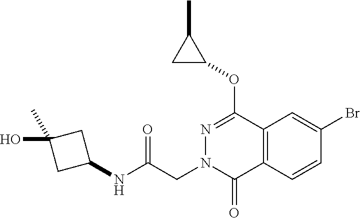 first eluting isomer |
| 8 | 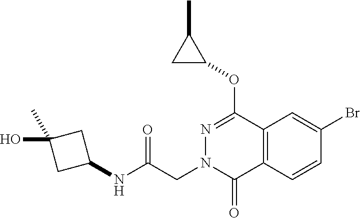 second eluting isomer |
| 9 | 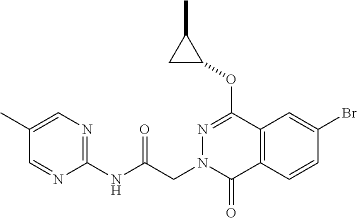 first eluting isomer |
| 10 | 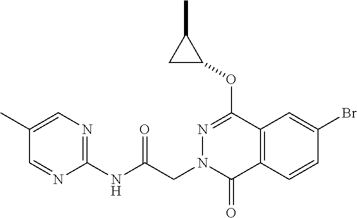 second eluting isomer |
| 11 | 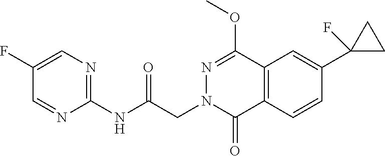 [SMILES: COc1nn(CC(=O)Nc2ncc(F)cn2)c(=O)c2ccc(C3(F)CC3)cc12] |
| 12 | 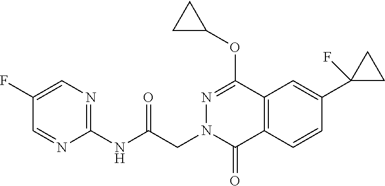 [SMILES: O=C(Cn1nc(OC2CC2)c2cc(C3(F)CC3)ccc2c1=O)Nc1ncc(F)cn1] |
| 13 | 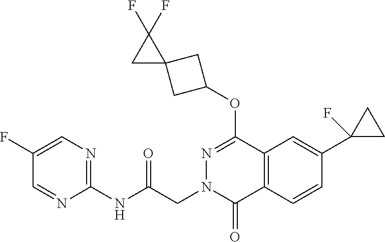 [SMILES: O=C(Cn1nc(OC2CC3(C2)CC3(F)F)c2cc(C3(F)CC3)ccc2c1=O)Nc1ncc(F)cn1] first eluting isomer |
| 14 | 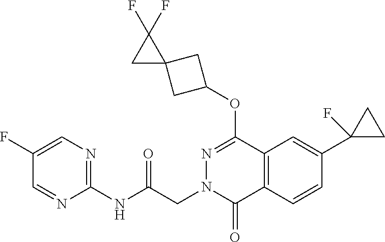 [SMILES: O=C(Cn1nc(OC2CC3(C2)CC3(F)F)c2cc(C3(F)CC3)ccc2c1=O)Nc1ncc(F)cn1] second eluting isomer |
| 15 | 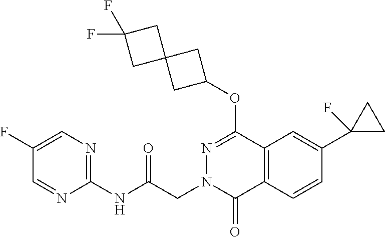 [SMILES: O=C(Cn1nc(OC2CC3(C2)CC(F)(F)C3)c2cc(C3(F)CC3)ccc2c1=O)Nc1ncc(F)cn1] |
| 16 | 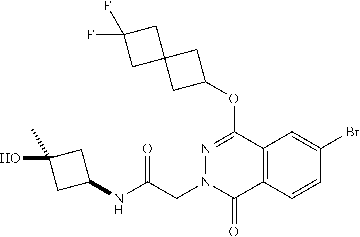 [SMILES: C[C@]1(O)C[C@@H](NC(=O)Cn2nc(OC3CC4(C3)CC(F)(F)C4)c3cc(Br)ccc3c2=O)C1] |
| 17 | 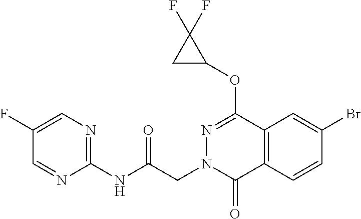 [SMILES: O=C(Cn1nc(OC2CC2(F)F)c2cc(Br)ccc2c1=O)Nc1ncc(F)cn1] first eluting isomer |
| 18 | 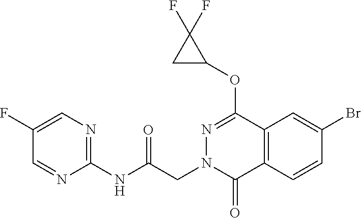 [SMILES: O=C(Cn1nc(OC2CC2(F)F)c2cc(Br)ccc2c1=O)Nc1ncc(F)cn1] second eluting isomer |
| 19 | 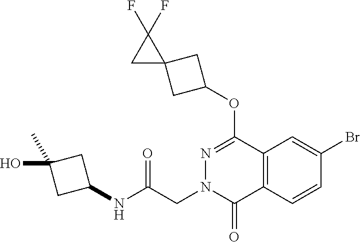 [SMILES: C[C@]1(O)C[C@@H](NC(=O)Cn2nc(OC3CC4(C3)CC4(F)F)c3cc(Br)ccc3c2=O)C1] first eluting isomer |
| 20 | 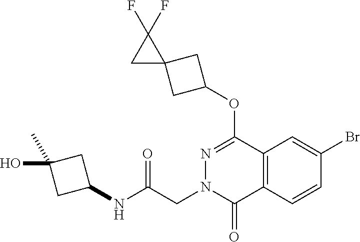 [SMILES: C[C@]1(O)C[C@@H](NC(=O)Cn2nc(OC3CC4(C3)CC4(F)F)c3cc(Br)ccc3c2=O)C1] second eluting isomer |
| 21 | 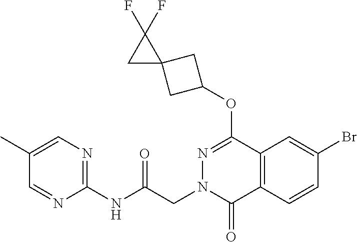 [SMILES: Cc1cnc(NC(=O)Cn2nc(OC3CC4(C3)CC4(F)F)c3cc(Br)ccc3c2=O)nc1] first eluting isomer |
| 22 | 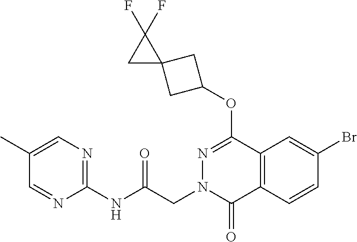 [SMILES: Cc1cnc(NC(=O)Cn2nc(OC3CC4(C3)CC4(F)F)c3cc(Br)ccc3c2=O)nc1] second eluting isomer |
| 23 | 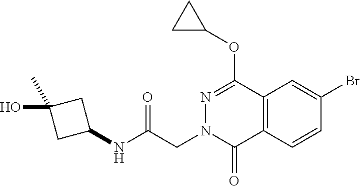 [SMILES: C[C@]1(O)C[C@@H](NC(=O)Cn2nc(OC3CC3)c3cc(Br)ccc3c2=O)C1] |
| 24 | 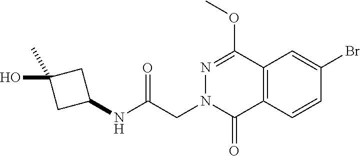 [SMILES: COc1nn(CC(=O)N[C@H]2C[C@@](C)(O)C2)c(=O)c2ccc(Br)cc12] |
| 25 | 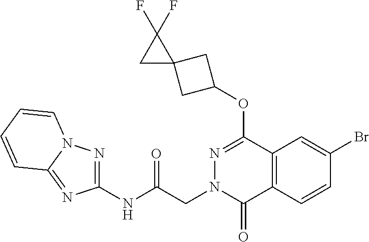 [SMILES: O=C(Cn1nc(OC2CC3(C2)CC3(F)F)c2cc(Br)ccc2c1=O)Nc1nc2ccccn2n1] first eluting isomer |
| 26 | 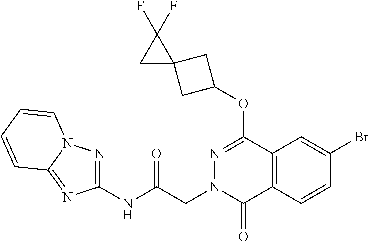 [SMILES: O=C(Cn1nc(OC2CC3(C2)CC3(F)F)c2cc(Br)ccc2c1=O)Nc1nc2ccccn2n1] second eluting isomer |
| 27 | 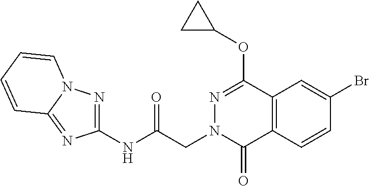 [SMILES: O=C(Cn1nc(OC2CC2)c2cc(Br)ccc2c1=O)Nc1nc2ccccn2n1] |
| 28 | 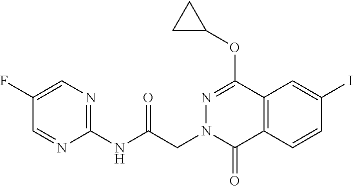 [SMILES: O=C(Cn1nc(OC2CC2)c2cc(I)ccc2c1=O)Nc1ncc(F)cn1] |
| 29 | 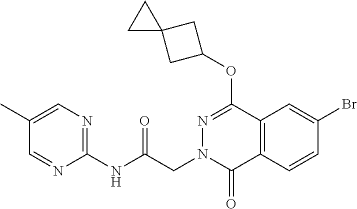 [SMILES: Cc1cnc(NC(=O)Cn2nc(OC3CC4(CC4)C3)c3cc(Br)ccc3c2=O)nc1] |
| 30 | 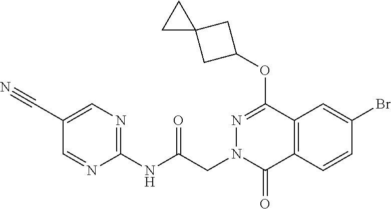 [SMILES: N#Cc1cnc(NC(=O)Cn2nc(OC3CC4(CC4)C3)c3cc(Br)ccc3c2=O)nc1] |
| 31 | 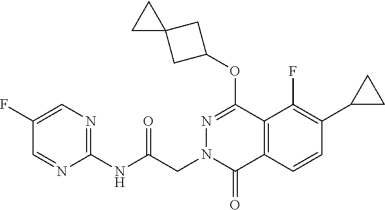 [SMILES: O=C(Cn1nc(OC2CC3(CC3)C2)c2c(F)c(C3CC3)ccc2c1=O)Nc1ncc(F)cn1] |
| 32 | 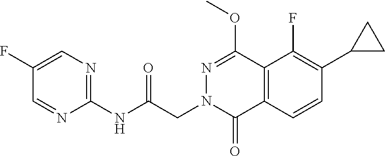 [SMILES: COc1nn(CC(=O)Nc2ncc(F)cn2)c(=O)c2ccc(C3CC3)c(F)c12] |
| 33 | 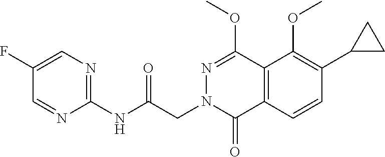 [SMILES: COc1nn(CC(=O)Nc2ncc(F)cn2)c(=O)c2ccc(C3CC3)c(OC)c12] |
| 34 | 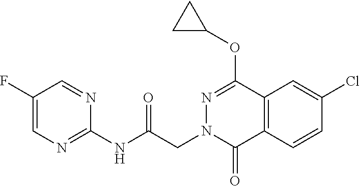 [SMILES: O=C(Cn1nc(OC2CC2)c2cc(Cl)ccc2c1=O)Nc1ncc(F)cn1] |
| 35 | 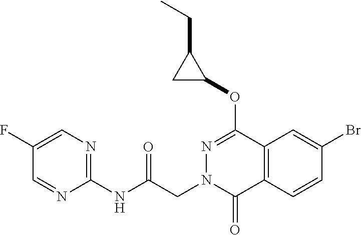 first eluting isomer |
| 36 | 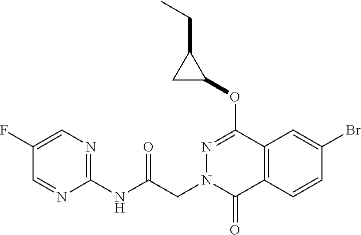 second eluting isomer |
| 37 | 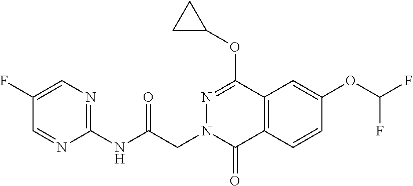 [SMILES: O=C(Cn1nc(OC2CC2)c2cc(OC(F)F)ccc2c1=O)Nc1ncc(F)cn1] |
| 38 | 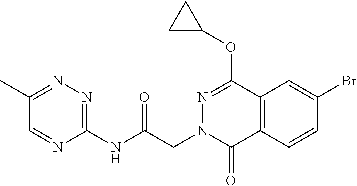 [SMILES: Cc1cnc(NC(=O)Cn2nc(OC3CC3)c3cc(Br)ccc3c2=O)nn1] |
| 39 | 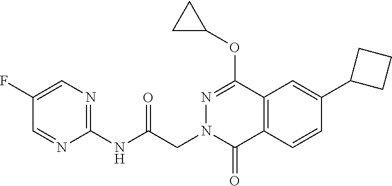 [SMILES: O=C(Cn1nc(OC2CC2)c2cc(C3CCC3)ccc2c1=O)Nc1ncc(F)cn1] |
| 40 | 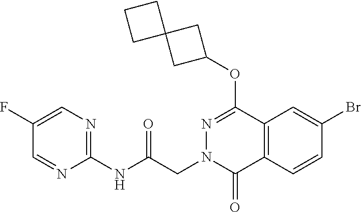 [SMILES: O=C(Cn1nc(OC2CC3(CCC3)C2)c2cc(Br)ccc2c1=O)Nc1ncc(F)cn1] |
| 41 | 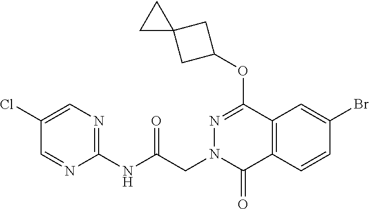 [SMILES: O=C(Cn1nc(OC2CC3(CC3)C2)c2cc(Br)ccc2c1=O)Nc1ncc(Cl)cn1] |
| 42 | 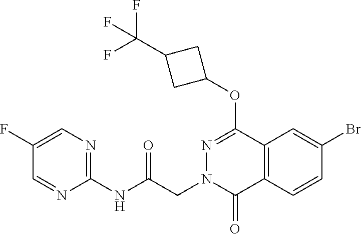 [SMILES: O=C(Cn1nc(OC2CC(C(F)(F)F)C2)c2cc(Br)ccc2c1=O)Nc1ncc(F)cn1] |
| 43 | 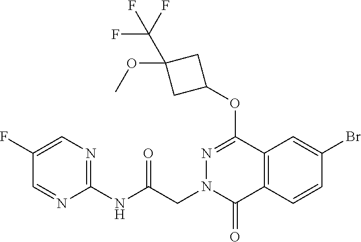 [SMILES: COC1(C(F)(F)F)CC(Oc2nn(CC(=O)Nc3ncc(F)cn3)c(=O)c3ccc(Br)cc23)C1] |
| 44 | 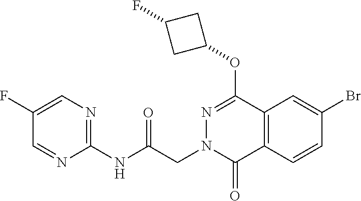 |
| 45 | 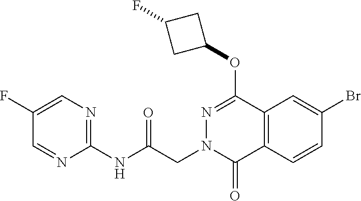 |
| 46 | 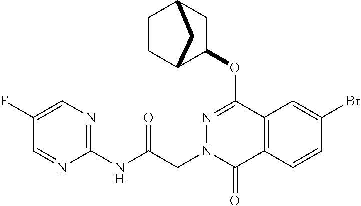 |
| 47 | 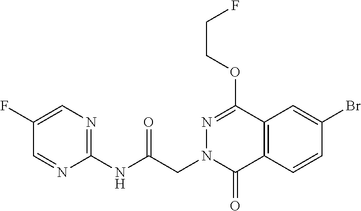 [SMILES: O=C(Cn1nc(OCCF)c2cc(Br)ccc2c1=O)Nc1ncc(F)cn1] |
| 48 | 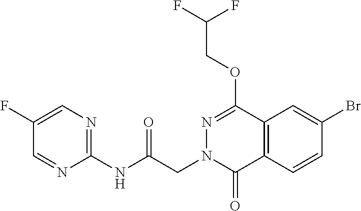 [SMILES: O=C(Cn1nc(OCC(F)F)c2cc(Br)ccc2c1=O)Nc1ncc(F)cn1] |
| 49 | 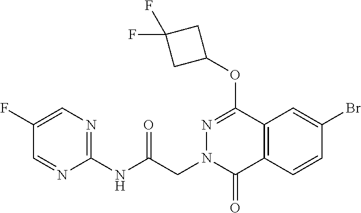 [SMILES: O=C(Cn1nc(OC2CC(F)(F)C2)c2cc(Br)ccc2c1=O)Nc1ncc(F)cn1] |
| 50 | 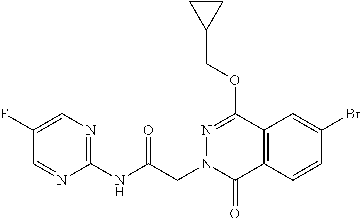 [SMILES: O=C(Cn1nc(OCC2CC2)c2cc(Br)ccc2c1=O)Nc1ncc(F)cn1] |
| 51 | 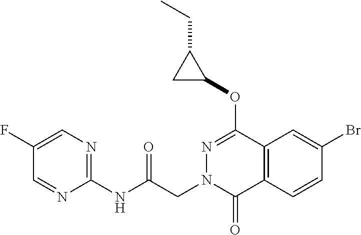 |
| 52 | 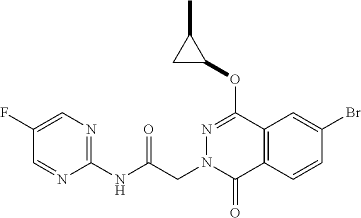 |
| 53 | 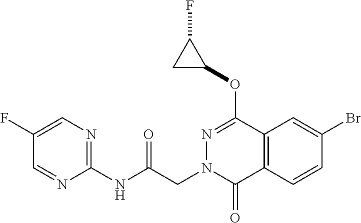 |
| 54 | 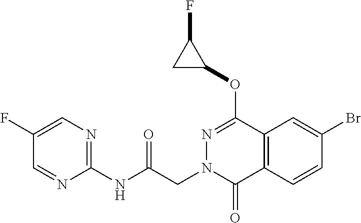 |

In certain embodiments, provided is a compound selected from Table 2 or a pharmaceutically acceptable salt, isotopically enriched analog, prodrug, stereoisomer, or a mixture of stereoisomers thereof:

TABLE 2

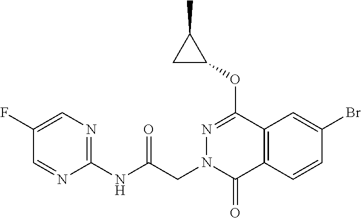 [SMILES: C[C@@H]1C[C@H]1Oc1nn(CC(=O)Nc2ncc(F)cn2)c(=O)c2ccc(Br)cc12]

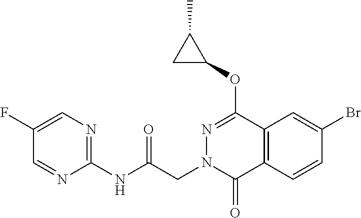 [SMILES: C[C@H]1C[C@@H]1Oc1nn(CC(=O)Nc2ncc(F)cn2)c(=O)c2ccc(Br)cc12]

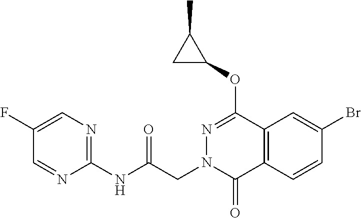 [SMILES: C[C@@H]1C[C@@H]1Oc1nn(CC(=O)Nc2ncc(F)cn2)c(=O)c2ccc(Br)cc12]

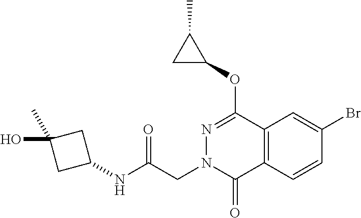 [SMILES: C[C@H]1C[C@@H]1Oc1nn(CC(=O)N[C@H]2C[C@](C)(O)C2)c(=O)c2ccc(Br)cc12]

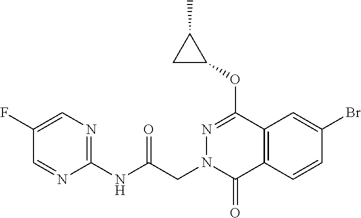 [SMILES: C[C@H]1C[C@H]1Oc1nn(CC(=O)Nc2ncc(F)cn2)c(=O)c2ccc(Br)cc12]

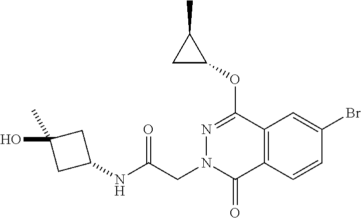 [SMILES: C[C@@H]1C[C@H]1Oc1nn(CC(=O)N[C@H]2C[C@](C)(O)C2)c(=O)c2ccc(Br)cc12]

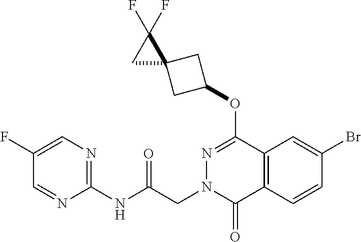

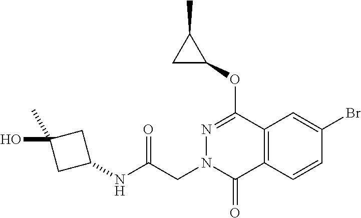 [SMILES: C[C@@H]1C[C@@H]1Oc1nn(CC(=O)N[C@H]2C[C@](C)(O)C2)c(=O)c2ccc(Br)cc12]

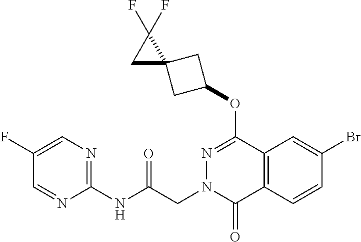

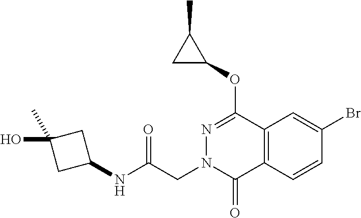 [SMILES: C[C@@H]1C[C@@H]1Oc1nn(CC(=O)N[C@H]2C[C@@](C)(O)C2)c(=O)c2ccc(Br)cc12]

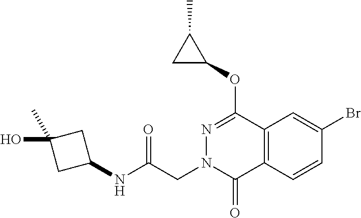 [SMILES: C[C@H]1C[C@@H]1Oc1nn(CC(=O)N[C@H]2C[C@@](C)(O)C2)c(=O)c2ccc(Br)cc12]

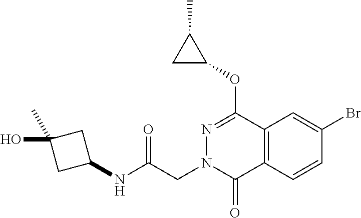 [SMILES: C[C@H]1C[C@H]1Oc1nn(CC(=O)N[C@H]2C[C@@](C)(O)C2)c(=O)c2ccc(Br)cc12]

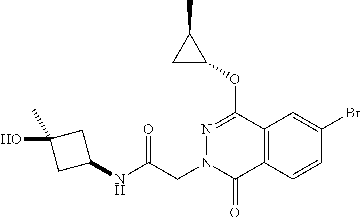 [SMILES: C[C@@H]1C[C@H]1Oc1nn(CC(=O)N[C@H]2C[C@@](C)(O)C2)c(=O)c2ccc(Br)cc12]

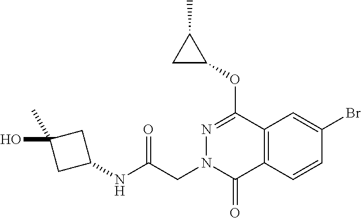 [SMILES: C[C@H]1C[C@H]1Oc1nn(CC(=O)N[C@H]2C[C@](C)(O)C2)c(=O)c2ccc(Br)cc12]

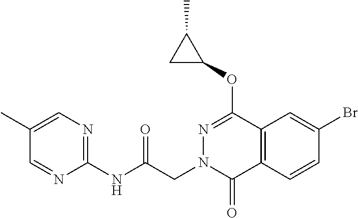 [SMILES: Cc1cnc(NC(=O)Cn2nc(O[C@H]3C[C@@H]3C)c3cc(Br)ccc3c2=O)nc1]

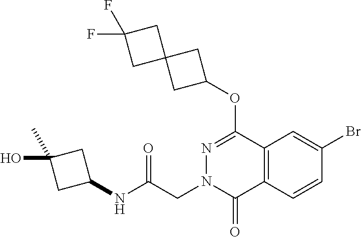 [SMILES: C[C@]1(O)C[C@@H](NC(=O)Cn2nc(OC3CC4(C3)CC(F)(F)C4)c3cc(Br)ccc3c2=O)C1]

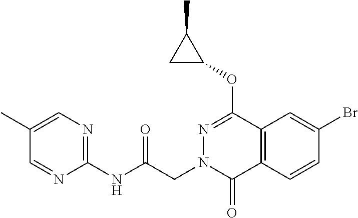 [SMILES: Cc1cnc(NC(=O)Cn2nc(O[C@@H]3C[C@H]3C)c3cc(Br)ccc3c2=O)nc1]

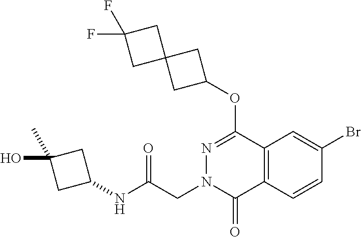 [SMILES: C[C@]1(O)C[C@H](NC(=O)Cn2nc(OC3CC4(C3)CC(F)(F)C4)c3cc(Br)ccc3c2=O)C1]

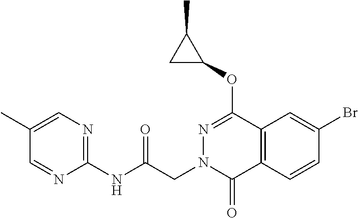 [SMILES: Cc1cnc(NC(=O)Cn2nc(O[C@H]3C[C@H]3C)c3cc(Br)ccc3c2=O)nc1]

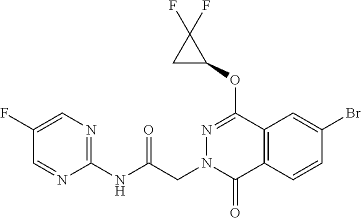 [SMILES: O=C(Cn1nc(O[C@H]2CC2(F)F)c2cc(Br)ccc2c1=O)Nc1ncc(F)cn1]

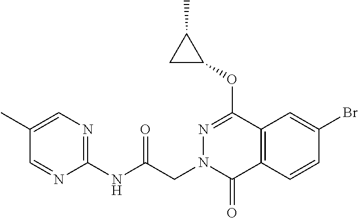 [SMILES: Cc1cnc(NC(=O)Cn2nc(O[C@@H]3C[C@@H]3C)c3cc(Br)ccc3c2=O)nc1]

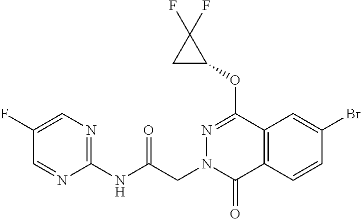 [SMILES: O=C(Cn1nc(O[C@@H]2CC2(F)F)c2cc(Br)ccc2c1=O)Nc1ncc(F)cn1]

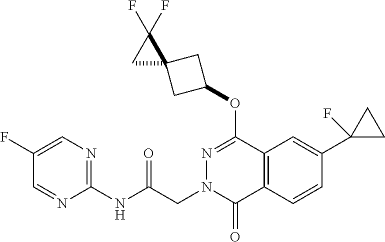

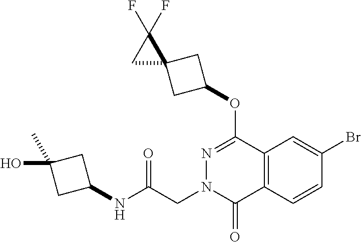

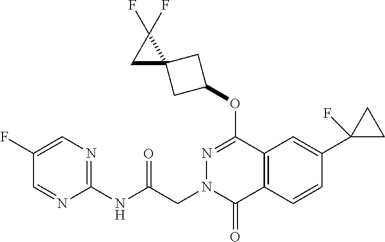

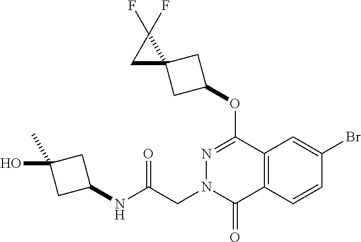

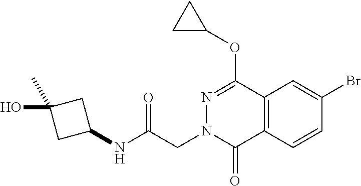 [SMILES: C[C@]1(O)C[C@@H](NC(=O)Cn2nc(OC3CC3)c3cc(Br)ccc3c2=O)C1]

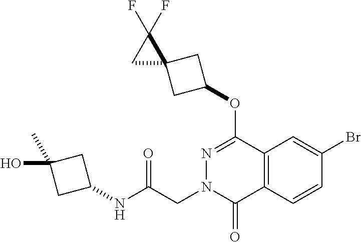

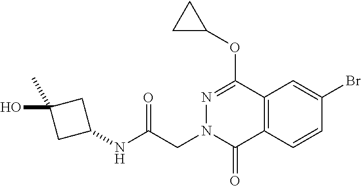 [SMILES: C[C@]1(O)C[C@H](NC(=O)Cn2nc(OC3CC3)c3cc(Br)ccc3c2=O)C1]

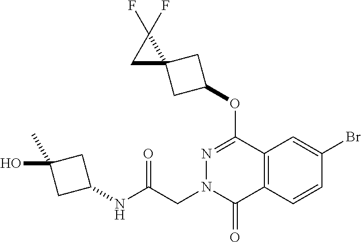

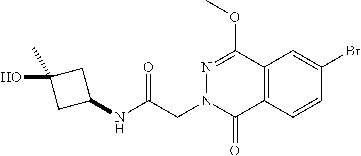 [SMILES: COc1nn(CC(=O)N[C@H]2C[C@@](C)(O)C2)c(=O)c2ccc(Br)cc12]

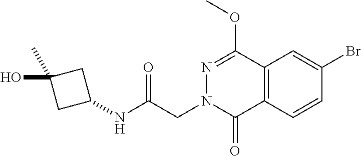 [SMILES: COc1nn(CC(=O)N[C@H]2C[C@](C)(O)C2)c(=O)c2ccc(Br)cc12]

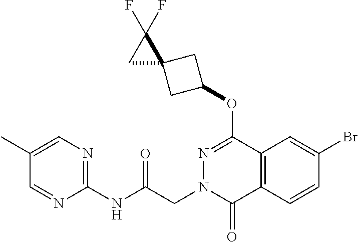

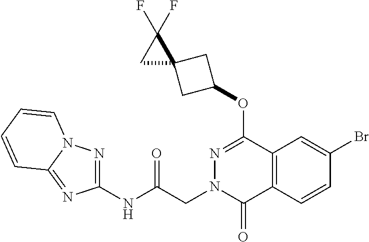

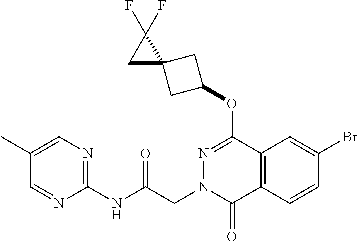

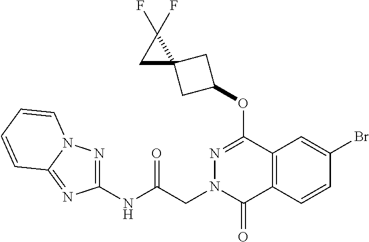

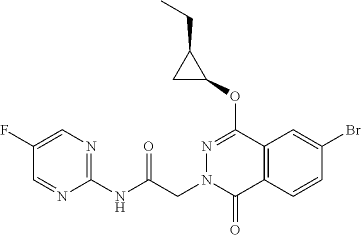 [SMILES: CC[C@@H]1C[C@@H]1Oc1nn(CC(=O)Nc2ncc(F)cn2)c(=O)c2ccc(Br)cc12]

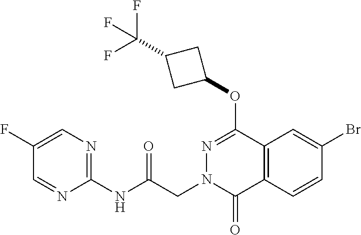

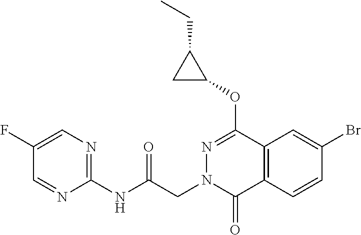 [SMILES: CC[C@H]1C[C@H]1Oc1nn(CC(=O)Nc2ncc(F)cn2)c(=O)c2ccc(Br)cc12]

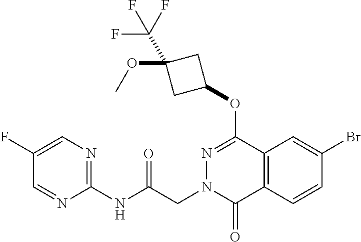

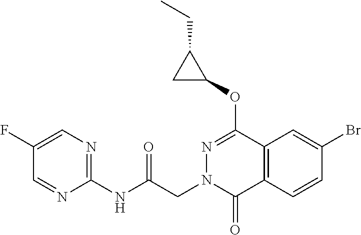 [SMILES: CC[C@H]1C[C@@H]1Oc1nn(CC(=O)Nc2ncc(F)cn2)c(=O)c2ccc(Br)cc12]

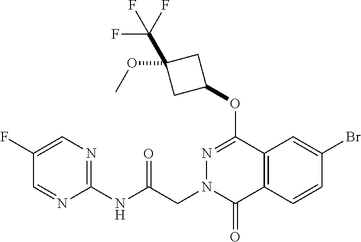

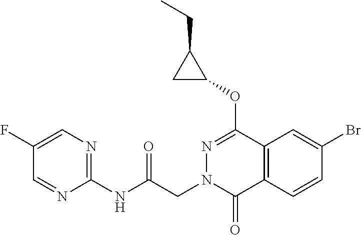 [SMILES: CC[C@@H]1C[C@H]1Oc1nn(CC(=O)Nc2ncc(F)cn2)c(=O)c2ccc(Br)cc12]

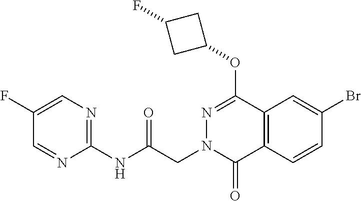

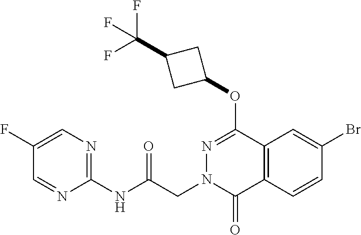

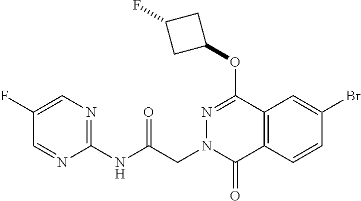

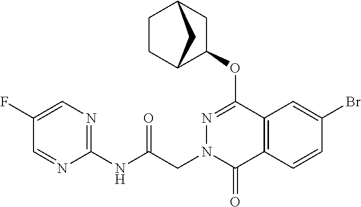 [SMILES: O=C(Cn1nc(O[C@@H]2C[C@H]3CC[C@@H]2C3)c2cc(Br)ccc2c1=O)Nc1ncc(F)cn1]

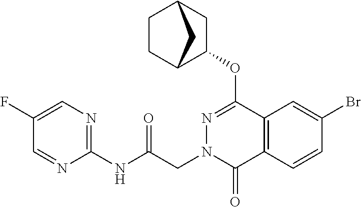 [SMILES: O=C(Cn1nc(O[C@H]2C[C@H]3CC[C@@H]2C3)c2cc(Br)ccc2c1=O)Nc1ncc(F)cn1]

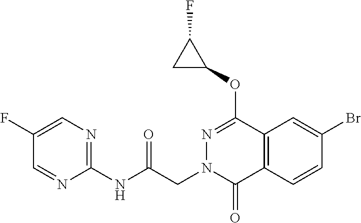 [SMILES: O=C(Cn1nc(O[C@H]2C[C@@H]2F)c2cc(Br)ccc2c1=O)Nc1ncc(F)cn1]

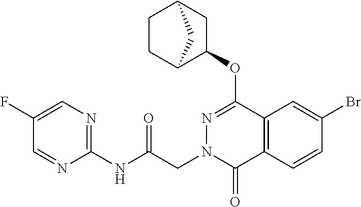 [SMILES: O=C(Cn1nc(O[C@@H]2C[C@@H]3CC[C@H]2C3)c2cc(Br)ccc2c1=O)Nc1ncc(F)cn1]

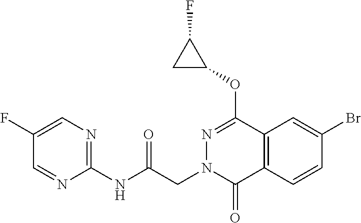 [SMILES: O=C(Cn1nc(O[C@@H]2C[C@@H]2F)c2cc(Br)ccc2c1=O)Nc1ncc(F)cn1]

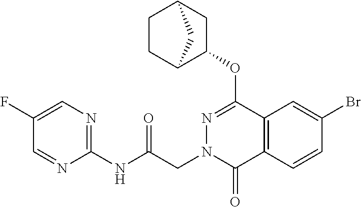 [SMILES: O=C(Cn1nc(O[C@H]2C[C@@H]3CC[C@H]2C3)c2cc(Br)ccc2c1=O)Nc1ncc(F)cn1]

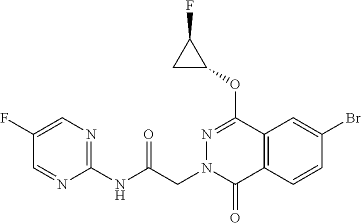 [SMILES: O=C(Cn1nc(O[C@@H]2C[C@H]2F)c2cc(Br)ccc2c1=O)Nc1ncc(F)cn1]

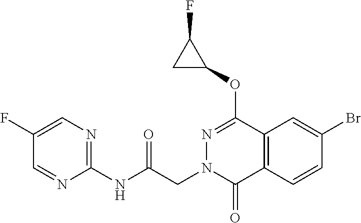 [SMILES: O=C(Cn1nc(O[C@H]2C[C@H]2F)c2cc(Br)ccc2c1=O)Nc1ncc(F)cn1]

### 3. Methods

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy, and/or veterinary applications. In some embodiments, the subject is a mammal. In certain embodiments, the subject is a human.

The term “therapeutically effective amount” or “effective amount” of a compound described herein or a pharmaceutically acceptable salt, isotopically enriched analog, stereoisomer, mixture of stereoisomers, or prodrug thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition of as described herein. The therapeutically effective amount may vary depending on the subject, and disease or condition being treated, the weight and age of the subject, the severity of the disease or condition, and the manner of administering, which can readily be determined by one of ordinary skill in the art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The compounds may be further characterized to examine the safety or tolerance dosage in human or non-human subjects. Such properties may be examined using commonly known methods to those skilled in the art.

In certain embodiments, provided are compounds, or a pharmaceutically acceptable salt, isotopically enriched analog, stereoisomer, mixture of stereoisomers, or prodrug thereof, that modulate the activity of NLR Family Pyrin Domain Containing 3 (NLRP3). In certain embodiments, the compounds provided herein, or a pharmaceutically acceptable salt, isotopically enriched analog, stereoisomer, mixture of stereoisomers, or prodrug thereof, inhibit the activation of NLRP3.

NLR proteins are involved in the immune system, helping to start and regulate the immune system's response to injury, toxins, or invasion by microorganisms. NLRP3 (also known as cryopyrin, NALP3, LRR and PYD domains-containing protein 3), is a protein encoded by the NLRP3 gene (also known as CIAS1). Once activated, NLRP3 molecules assemble, along with other proteins, into inflammasomes. The activation of NLRP3 by cellular stress leads to inflammasome activation and downstream proteolytic events, including the formation of active proinflammatory cytokines such as interleukin (IL)-1β and IL-18 which are then secreted. Among other cytokines, IL-1β and IL-18 are known mediators of inflammation, e.g., artery wall inflammation, atherosclerosis and the aging process.

In certain embodiments, provided is a method of inhibiting inflammasome (e.g., the NLRP3 inflammasome) activity comprising contacting a cell with an effective amount of a compound disclosed herein, or a pharmaceutically acceptable salt, isotopically enriched analog, stereoisomer, mixture of stereoisomers, or prodrug thereof. The inhibiting can be in vitro or in vivo.

In certain embodiments, provided is a compound as disclosed herein, or a pharmaceutically acceptable salt, isotopically enriched analog, stereoisomer, mixture of stereoisomers, or prodrug thereof, for use in inhibiting inflammasome (e.g., the NLRP3 inflammasome) activity (e.g., in vitro or in vivo).

In certain embodiments, the present disclosure provides use of a compound as disclosed herein, or a pharmaceutically acceptable salt, isotopically enriched analog, stereoisomer, mixture of stereoisomers, or prodrug thereof, in the manufacture of a medicament for inhibiting inflammasome (e.g., the NLRP3 inflammasome) activity (e.g., in vitro or in vivo).

Chronic inflammation responses have been associated with various types of cancer. During malignant transformation or cancer therapy, inflammasomes may become activated in response to certain signals; and IL-Iβ expression is elevated in a variety of cancers (e.g., breast, prostate, colon, lung, head and neck cancers, melanomas, etc.), where patients with IL-Iβ producing tumors generally have a worse prognosis.

In certain embodiments, provided is a method for treating a disease or condition mediated, at least in part, by NLRP3, comprising administering an effective amount of a compound disclosed herein, or a pharmaceutically acceptable salt, isotopically enriched analog, stereoisomer, mixture of stereoisomers, or prodrug thereof, to a subject in need thereof.

In certain embodiments, provided is a method for treating a disease or condition selected from an autoinflammatory disorder, an autoimmune disorder, a neurodegenerative disease or cancer, comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a compound as disclosed herein, or a pharmaceutically acceptable salt, isotopically enriched analog, stereoisomer, mixture of stereoisomers, or prodrug thereof, for use in treating an autoinflammatory disorder, an autoimmune disorder, a neurodegenerative disease or cancer in a subject in need thereof.

In certain embodiments, the present disclosure provides use of a compound as disclosed herein, or a pharmaceutically acceptable salt, isotopically enriched analog, stereoisomer, mixture of stereoisomers, or prodrug thereof in the manufacture of a medicament for treating or preventing an autoinflammatory disorder, an autoimmune disorder, a neurodegenerative disease or cancer in a subject in need thereof.

In certain embodiments, provided is a method for treating inflammation, an auto-immune disease, cancer, an infection, a central nervous system disease, a metabolic disease, a cardiovascular disease, a respiratory disease, a liver disease, a renal disease, an ocular disease, a skin disease, a lymphatic condition, a psychological disorder, graft versus host disease, allodynia, and any disease where an individual has been determined to carry a germline or somatic non-silent mutation in NLRP3, comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, the disease or condition may be a disease or condition of the immune system, the cardiovascular system, the endocrine system, the gastrointestinal tract, the renal system, the hepatic system, the metabolic system, the respiratory system, the central nervous system, may be a cancer or other malignancy, and/or may be caused by or associated with a pathogen. It will be appreciated that these general embodiments defined according to broad categories of diseases, disorders and conditions are not mutually exclusive.

In certain embodiments, the disease or condition includes, inflammation, including inflammation occurring as a result of an inflammatory disorder, e.g. an autoinflammatory disease, inflammation occurring as a symptom of a non-inflammatory disorder, inflammation occurring as a result of infection, or inflammation secondary to trauma, injury or autoimmunity; auto-immune diseases such as acute disseminated encephalitis, Addison's disease, ankylosing spondylitis, antiphospholipid antibody syndrome (APS), anti-synthetase syndrome, aplastic anemia, autoimmune adrenalitis, autoimmune hepatitis, autoimmune oophoritis, autoimmune polyglandular failure, autoimmune thyroiditis, Coeliac disease, Crohn's disease, type 1 diabetes (TID), Goodpasture's syndrome, Graves' disease, Guillain-Barré syndrome (GBS), Hashimoto's disease, idiopathic thrombocytopenic purpura, Kawasaki's disease, lupus erythematosus including systemic lupus erythematosus (SLE), multiple sclerosis (MS) including primary progressive multiple sclerosis (PPMS), secondary progressive multiple sclerosis (SPMS) and relapsing remitting multiple sclerosis (RRMS), myasthenia gravis, opsoclonus myoclonus syndrome (OMS), optic neuritis, Ord's thyroiditis, pemphigus, pernicious anemia, polyarthritis, primary biliary cirrhosis, rheumatoid arthritis (RA), psoriatic arthritis, juvenile idiopathic arthritis or Still's disease, refractory gouty arthritis, Reiter's syndrome, Sjögren's syndrome, systemic sclerosis a systemic connective tissue disorder, Takayasu's arteritis, temporal arteritis, warm autoimmune hemolytic anemia, Wegener's granulomatosis, alopecia universalis, Behçet's disease, Chagas' disease, dysautonomia, endometriosis, hidradenitis suppurativa (HS), interstitial cystitis, neuromyotonia, psoriasis, sarcoidosis, scleroderma, ulcerative colitis, Schnitzler syndrome, macrophage activation syndrome, Blau syndrome, vitiligo or vulvodynia; cancer including lung cancer, pancreatic cancer, gastric cancer, myelodysplastic syndrome, leukemia including acute lymphocytic leukemia (ALL) and acute myeloid leukemia (AML), adrenal cancer, anal cancer, basal and squamous cell skin cancer, bile duct cancer, bladder cancer, bone cancer, brain and spinal cord tumors, breast cancer, cervical cancer, chronic lymphocytic leukemia (CLL), chronic myeloid leukemia (CML), chronic myelomonocytic leukemia (CMML), colorectal cancer, endometrial cancer, oesophagus cancer, Ewing family of tumors, eye cancer, gallbladder cancer, gastrointestinal carcinoid tumors, gastrointestinal stromal tumor (GIST), gestational trophoblastic disease, glioma, Hodgkin lymphoma, Kaposi sarcoma, kidney cancer, laryngeal and hypopharyngeal cancer, liver cancer, lung carcinoid tumor, lymphoma including cutaneous T cell lymphoma, malignant mesothelioma, melanoma skin cancer, Merkel cell skin cancer, multiple myeloma, nasal cavity and paranasal sinuses cancer, nasopharyngeal cancer, neuroblastoma, non-Hodgkin lymphoma, non-small cell lung cancer, oral cavity and oropharyngeal cancer, osteosarcoma, ovarian cancer, penile cancer, pituitary tumors, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer including anaplastic thyroid cancer, uterine sarcoma, vaginal cancer, vulvar cancer, Waldenstrom macroglobulinemia, and Wilms tumor; infections including viral infections (e.g. from influenza virus, human immunodeficiency virus (HIV), alphavirus (such as Chikungunya and Ross River virus), flaviviruses (such as Dengue virus and Zika virus), herpes viruses (such as Epstein Barr Virus, cytomegalovirus, Varicella-zoster virus, and KSHV), poxviruses (such as vaccinia virus (Modified vaccinia virus Ankara) and Myxoma virus), adenoviruses (such as Adenovirus 5), or papillomavirus), bacterial infections (e.g. from Staphylococcus aureus, Helicobacter pylori, Bacillus anthracis, Bordatella pertussis, Burkholderia pseudomallei, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi or Yersinia pestis), fungal infections (e.g. from Candida or Aspergillus species), protozoan infections (e.g. from Plasmodium, Babesia, Giardia, Entamoeba, Leishmania or Trypanosomes), helminth infections (e.g. from schistosoma, roundworms, tapeworms or flukes) and prion infections; central nervous system diseases such as Parkinson's disease, Alzheimer's disease, dementia, motor neuron disease, Huntington's disease, cerebral malaria, brain injury from pneumococcal meningitis, intracranial aneurysms, traumatic brain injury, and amyotrophic lateral sclerosis; metabolic diseases such as type 2 diabetes (T2D), atherosclerosis, obesity, gout, and pseudo-gout; cardiovascular diseases such as hypertension, ischemia, reperfusion injury including post-MI ischemic reperfusion injury, stroke including ischemic stroke, transient ischemic attack, myocardial infarction including recurrent myocardial infarction, heart failure including congestive heart failure and heart failure with preserved ejection fraction, embolism, aneurysms including abdominal aortic aneurysm, and pericarditis including Dressler's syndrome; respiratory diseases including chronic obstructive pulmonary disorder (COPD), asthma such as allergic asthma and steroid-resistant asthma, asbestosis, silicosis, nanoparticle induced inflammation, cystic fibrosis and idiopathic pulmonary fibrosis; liver diseases including non-alcoholic fatty liver disease (NAFLD) and non-alcoholic steatohepatitis (NASH) including advanced fibrosis stages F3 and F4; alcoholic fatty liver disease (AFLD), and alcoholic steatohepatitis (ASH); renal diseases including chronic kidney disease, oxalate nephropathy, nephrocalcinosis, glomerulonephritis, and diabetic nephropathy; ocular diseases including those of the ocular epithelium, age-related macular degeneration (AMD) (dry and wet), uveitis, corneal infection, diabetic retinopathy, optic nerve damage, dry eye, and glaucoma; skin diseases including dermatitis such as contact dermatitis and atopic dermatitis, contact hypersensitivity, sunburn, skin lesions, hidradenitis suppurativa (HS), other cyst-causing skin diseases, and acne conglobata; lymphatic conditions such as lymphangitis and Castleman's disease; psychological disorders such as depression and psychological stress; graft versus host disease; allodynia including mechanical allodynia; and any disease where an individual has been determined to carry a germline or somatic non-silent mutation in NLRP3.

In certain embodiments, the disease, disorder or condition is an autoinflammatory disease such as cryopyrin-associated periodic syndromes (CAPS), Muckle-Wells syndrome (MWS), familial cold autoinflammatory syndrome (FCAS), familial Mediterranean fever (FMF), neonatal onset multisystem inflammatory disease (NOMID), tumor Necrosis Factor (TNF) Receptor-Associated Periodic Syndrome (TRAPS), hyperimmunoglobulinemia D and periodic fever syndrome (HIDS), deficiency of interleukin 1 receptor antagonist (DIRA), Majeed syndrome, pyogenic arthritis, pyoderma gangrenosum and acne syndrome (PAPA), adult-onset Still's disease (AOSD), haploinsufficiency of A20 (HA20), pediatric granulomatous arthritis (PGA), PLCG2-associated antibody deficiency and immune dysregulation (PLAID), PLCG2-associated autoinflammatory, antibody deficiency and immune dysregulation (APLAID), or sideroblastic anemia with B-cell immunodeficiency, periodic fevers, and developmental delay (SIFD).

In certain embodiments, provided is a method for treating a disease or condition selected from an autoinflammatory disorder and/or an autoimmune disorder selected from cryopyrin-associated autoinflammatory syndrome (CAPS; e.g., familial cold autoinflammatory syndrome (FCAS)), Muckle-Wells syndrome (MWS), chronic infantile neurological cutaneous and articular (CINCA) syndrome, neonatal-onset multisystem inflammatory disease (NOMID), familial Mediterranean fever and nonalcoholic fatty liver disease (NAFLD), non-alcoholic steatohepatitis (NASH), gout, rheumatoid arthritis, osteoarthritis, Crohn's disease, chronic obstructive pulmonary disease (COPD), chronic kidney disease (CKD), fibrosis, obesity, type 2 diabetes, and multiple sclerosis and neuroinflammation occurring in protein misfolding diseases (e.g., Prion diseases) comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a method for treating a disease or condition selected from cryopyrin-associated periodic syndromes (CAPS), Muckle-Wells syndrome (MWS), familial cold autoinflammatory syndrome (FCAS), neonatal onset multisystem inflammatory disease (NOMID), familial Mediterranean fever (FMF), pyogenic arthritis, pyoderma gangrenosum and acne syndrome (PAPA); hyperimmunoglobulinemia D and periodic fever syndrome (HIDS), Tumor Necrosis Factor (TNF), Receptor-Associated Periodic Syndrome (TRAPS), systemic juvenile idiopathic arthritis, adult-onset Still's disease (AOSD), relapsing polychondritis, Schnitzler's syndrome, Sweet's syndrome, Behcet's disease, anti-synthetase syndrome, deficiency of interleukin 1 receptor antagonist (DIRA), and haploinsufficiency of A20 (HA20) comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a method for treating a disease or condition selected from Alzheimer's disease, atherosclerosis, asthma, allergic airway inflammation, cryopyrin-associated periodic syndromes, gout, inflammatory bowel disease and related disorders, nonalcoholic fatty liver disease (NAFLD), nonalcoholic steatohepatitis (NASH), hypertension, myocardial infarction, multiple sclerosis, experimental autoimmune encephalitis, oxalate-induced nephropathy, hyperinflammation following influenza infection, graft-versus-host disease, stroke, silicosis, type 1 diabetes, obesity-induced inflammation or insulin resistance, rheumatoid arthritis, myelodysplastic syndrome, contact hypersensitivity, joint inflammation triggered by chikungunya virus, or traumatic brain injury comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a method for treating a disease or condition that is mediated, at least in part, by TNF-α. In certain embodiments, the disease or condition is resistant to treatment with an anti-TNF-α agent. In some embodiments, the disease is a gut disease or condition. In some embodiments the disease or condition is inflammatory bowel disease, Crohn's disease, or ulcerative colitis. In some embodiments, a compound disclosed herein or a pharmaceutically acceptable salt, isotopically enriched analog, stereoisomer, mixture of stereoisomers, or prodrug thereof is administered in combination with an anti-TNF-α agent. In some embodiments, the anti-TNF-α agent is infliximab, etanercept, certolizumab pegol, golimumab, or adalimumab.

In certain embodiments, the disease or condition is an autoinflammatory disorder, an autoimmune disorder, a neurodegenerative disease, or cancer.

In certain embodiments, the disease or condition is an autoinflammatory disorder and/or an autoimmune disorder.

In certain embodiments, the disease or condition is a neurodegenerative disease.

In certain embodiments, the disease or condition is Parkinson's disease or Alzheimer's disease.

In certain embodiments, provided is a method for treating cancer, comprising administering an effective amount of a compound disclosed herein, or a pharmaceutically acceptable salt, isotopically enriched analog, stereoisomer, mixture of stereoisomers, or prodrug thereof, to a subject in need thereof.

In certain embodiments, the cancer is metastasizing cancer, gastrointestinal cancer, skin cancer, non-small-cell lung carcinoma, or colorectal adenocarcinoma.

In certain embodiments, provided is a compound as disclosed herein, or a pharmaceutically acceptable salt, isotopically enriched analog, stereoisomer, mixture of stereoisomers, or prodrug thereof for use in treating a neurodegenerative disease (e.g., Parkinson's disease or Alzheimer's disease) in a subject in need thereof.

In certain embodiments, provided is a compound as disclosed herein, or a pharmaceutically acceptable salt, isotopically enriched analog, stereoisomer, mixture of stereoisomers, or prodrug thereof, for use in treating cancer in a subject in need thereof.

In certain embodiments, a compound as disclosed herein, or a pharmaceutically acceptable salt, isotopically enriched analog, stereoisomer, mixture of stereoisomers, or prodrug thereof, may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e., by itself the adjuvant may only have minimal therapeutic benefit, but in combination with another therapeutic agent, the overall therapeutic benefit to the individual is enhanced). Alternatively, by way of example only, the benefit experienced by an individual may be increased by administering compound as disclosed herein, or a pharmaceutically acceptable salt, isotopically enriched analog, stereoisomer, mixture of stereoisomers, or prodrug thereof, with another therapeutic agent (which also includes a therapeutic regimen) that also has therapeutic benefit.

Other embodiments include use of the presently disclosed compounds in therapy.

### 4. Kits

Provided herein are also kits that include a compound of the disclosure, or a pharmaceutically acceptable salt, isotopically enriched analog, stereoisomer, mixture of stereoisomers, or prodrug thereof, and suitable packaging. In certain embodiments, a kit further includes instructions for use. In one aspect, a kit includes a compound of the disclosure, or a pharmaceutically acceptable salt, isotopically enriched analog, stereoisomer, mixture of stereoisomers, or prodrug thereof, and a label and/or instructions for use of the compounds in the treatment of the indications, including the diseases or conditions, described herein.

Provided herein are also articles of manufacture that include a compound described herein or a pharmaceutically acceptable salt, isotopically enriched analog, stereoisomer, mixture of stereoisomers, or prodrug thereof in a suitable container. The container may be a vial, jar, ampoule, preloaded syringe, or intravenous bag.

### 5. Pharmaceutical Compositions and Modes of Administration

Compounds provided herein are usually administered in the form of pharmaceutical compositions. Thus, provided herein are also pharmaceutical compositions that contain one or more of the compounds described herein, or a pharmaceutically acceptable salt, stereoisomer, mixture of stereoisomers, or prodrug thereof, and one or more pharmaceutically acceptable vehicles selected from carriers, adjuvants, and excipients. Suitable pharmaceutically acceptable vehicles may include, for example, inert solid diluents and fillers, diluents, including sterile aqueous solution and various organic solvents, permeation enhancers, solubilizers, and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal, and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, isotopically enriched analog, stereoisomer, mixture of stereoisomers, or prodrug thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include, e.g., lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, isotopically enriched analog, stereoisomer, mixture of stereoisomers, or prodrug thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, isotopically enriched analog, stereoisomer, mixture of stereoisomers, or prodrug thereof. When referring to these preformulation compositions as homogeneous, the active ingredient may be dispersed evenly throughout the composition so that the composition may be readily subdivided into equally effective unit dosage forms such as tablets, pills, and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, orally or nasally, from devices that deliver the formulation in an appropriate manner.

### 6. Dosing

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. In some embodiments, a dosage of from about 0.0001 to about 100 mg per kg of body weight per day, from about 0.001 to about 50 mg of compound per kg of body weight, or from about 0.01 to about 10 mg of compound per kg of body weight may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

### 7. Synthesis of the Compounds

The compounds may be prepared using the methods disclosed herein and routine modifications thereof, which will be apparent given the disclosure herein and methods well known in the art. Conventional and well-known synthetic methods may be used in addition to the teachings herein. The synthesis of typical compounds described herein may be accomplished as described in the following examples. If available, reagents and starting materials may be purchased commercially, e.g., from Sigma Aldrich or other chemical suppliers.

It will be appreciated that where typical process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, conventional protecting groups (“PG”) may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006). Greene's protective groups in organic synthesis. Hoboken, N.J., Wiley-Interscience, and references cited therein. For example, protecting groups for alcohols, such as hydroxy, include silyl ethers (including trimethylsilyl (TMS), tert-butyldimethylsilyl (TBDMS), tri-iso-propylsilyloxymethyl (TOM), and triisopropylsilyl (TIPS) ethers), which can be removed by acid or fluoride ion, such as NaF, TBAF (tetra-n-butylammonium fluoride), HF-Py, or HF-NEt₃. Other protecting groups for alcohols include acetyl, removed by acid or base, benzoyl, removed by acid or base, benzyl, removed by hydrogenation, methoxyethoxymethyl ether, removed by acid, dimethoxytrityl, removed by acid, methoxymethyl ether, removed by acid, tetrahydropyranyl or tetrahydrofuranyl, removed by acid, and trityl, removed by acid. Examples of protecting groups for amines include carbobenzyloxy, removed by hydrogenolysis p-methoxybenzyl carbonyl, removed by hydrogenolysis, tert-butyloxycarbonyl, removed by concentrated strong acid (such as HCl or CF₃COOH), or by heating to greater than about 80° C., 9-fluorenylmethyloxycarbonyl, removed by base, such as piperidine, acetyl, removed by treatment with a base, benzoyl, removed by treatment with a base, benzyl, removed by hydrogenolysis, carbamate group, removed by acid and mild heating, p-methoxybenzyl, removed by hydrogenolysis, 3,4-dimethoxybenzyl, removed by hydrogenolysis, p-methoxyphenyl, removed by ammonium cerium(IV) nitrate, tosyl, removed by concentrated acid (such as HBr or H₂SO₄) and strong reducing agents (sodium in liquid ammonia or sodium naphthalenide), troc (trichloroethyl chloroformate), removed by Zn insertion in the presence of acetic acid, and sulfonamides (Nosyl & Nps), removed by samarium iodide or tributyltin hydride.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### General Experimental Methods

All solvents used were commercially available and were used without further purification. Reactions were typically run using anhydrous solvents under an inert atmosphere of nitrogen.

NMR Spectroscopy: ¹H Nuclear magnetic resonance (NMR) spectroscopy was carried out using a Bruker Avance III equipped with a BBFO 300 MHz probe operating at 300 MHz or one of the following instruments: a Bruker Avance 400 instrument equipped with probe DUAL 400 MHz Sl, a Bruker Avance 400 instrument equipped with probe 6 Si 400 MHz 5 mm ¹H-¹³C ID, a Bruker Avance III 400 instrument with nanobay equipped with probe Broadband BBFO 5 mm direct, a Bruker Mercury Plus 400 NMR spectrometer equipped with a Bruker 400 BBO probe operating at 400 MHz. All deuterated solvents contained typically 0.03% to 0.05% v/v tetramethylsilane, which was used as the reference signal (set at δ 0.00 for both ¹H and ¹³C). In certain cases, ¹H Nuclear magnetic resonance (NMR) spectroscopy was carried out using a Bruker Advance 400 instrument operating at 400 MHz using the stated solvent at around room temperature unless otherwise stated. In all cases, NMR data were consistent with the proposed structures. Characteristic chemical shifts (6) are given in parts-per-million using conventional abbreviations for designation of major peaks: e.g. s, singlet; d, doublet; t, triplet; q, quartet; dd, doublet of doublets; dt, doublet of triplets; br, broad.

Thin Layer Chromatography: Where thin layer chromatography (TLC) has been used it refers to silica gel TLC using silica gel F254 (Merck) plates, Rf is the distance travelled by the compound divided by the distance travelled by the solvent on a TLC plate. Column chromatography was performed using an automatic flash chromatography system over silica gel cartridges or in the case of reverse phase chromatography over C18 cartridges. Alternatively, thin layer chromatography (TLC) was performed on Alugram® (Silica gel 60 F254) from Mancherey-Nagel and UV was typically used to visualize the spots. Additional visualization methods were also employed in some cases. In these cases the TLC plate was developed with iodine (generated by adding approximately 1 g of I₂ to 10 g silica gel and thoroughly mixing), ninhydrin (available commercially from Aldrich), or Magic Stain (generated by thoroughly mixing 25 g (NH₄)₆Mo₇O₂₄·4H₂O, 5 g (NH₄)₂Ce(IV)(NO₃)₆ in 450 mL water and 50 mL concentrated H₂SO₄) to visualize the compound.

Liquid Chromatography-Mass Spectrometry and HPLC Analysis: HPLC analysis was performed on Shimadzu 20AB HPLC system with a photodiode array detector and Luna-C18(2) 2.0×50 mm, 5 μm column at a flow rate of 1.2 mL/min with a gradient solvent Mobile phase A (MPA, H₂O+0.037% (v/v) TFA): Mobile phase B (MPB, ACN+0.018% (v/v) TFA) (0.01 min, 10% MPB; 4 min, 80% MPB; 4.9 min, 80% MPB; 4.92 min, 10% MPB; 5.5 min, 10% MPB). LCMS was detected under 220 and 254 nm or used evaporative light scattering (ELSD) detection as well as positive electrospray ionization (MS). Semi-preparative HPLC was performed by either acidic or neutral conditions. Acidic: Luna C18 100×30 mm, 5 m; MPA: HCl/H₂O=0.04%, or formic acid/H₂O=0.2% (v/v); MPB: ACN. Neutral: Waters Xbridge 150×25, 5 m; MPA: 10 mM NH₄HCO₃ in H₂O; MPB: ACN. Gradient for both conditions: 10% of MPB to 80% of MPB over 12 min at a flow rate of 20 mL/min, then 100% MPB over 2 min, 10% MPB over 2 min, UV detector. SFC analysis was performed on Thar analytical SFC system with a UV/Vis detector and series of chiral columns including AD, AS-H, OJ, OD, AY and IC, 4.6×100 mm, 3 μm column at a flow rate of 4 mL/min with a gradient solvent Mobile phase A (MPA, CO₂): Mobile phase B (MPB, MeOH+0.05% (v/v) IPAm) (0.01 min, 10% MPB; 3 min, 40% MPB; 3.5 min, 40% MPB; 3.56-5 min, 10% MPB). SFC preparative was performed on Thar 80 preparative SFC system with a UV/Vis detector and series of chiral preparative columns including AD-H, AS-H, OJ-H, OD-H, AY-H and IC-H, 30×250 mm, 5 m column at a flow rate of 65 mL/min with a gradient solvent Mobile phase A (MPA, CO₂): Mobile phase B (MPB, MeOH+0.1% (v/v) NH₃H₂O) (0.01 min, 10% MPB; 5 min, 40% MPB; 6 min, 40% MPB; 6.1-10 min, 10% MPB). LC-MS data were also collected using an UPLC-MS Acquity™ system equipped with PDA detector and coupled to a Waters single quadrupole mass spectrometer operating in alternated positive and negative electrospray ionization mode. The column used was a Cortecs UPLC C18, 1.6 μm, 2.1×50 mm. A linear gradient was applied, starting at 95% A (A: 0.1% formic acid in water) and ending at 95% B (B: 0.1% formic acid in MeCN) over 2.0 min with a total run time of 2.5 min. The column temperature was at 40° C. with the flow rate of 0.8 mL/min.

### Intermediate 1

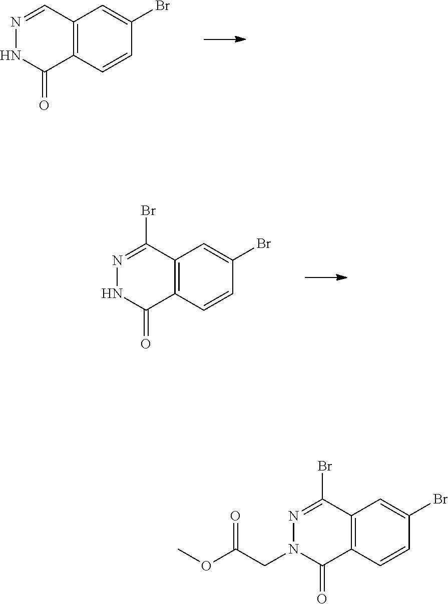 [SMILES: O=c1[nH]ncc2cc(Br)ccc12 ; O=c1[nH]nc(Br)c2cc(Br)ccc12 ; COC(=O)Cn1nc(Br)c2cc(Br)ccc2c1=O]

4,6-dibromo-2H-phthalazin-1-one: To a solution of 6-bromo-2H-phthalazin-1-one (1 g, 4.33 mmol) in DMF (30 mL) at 0° C. were added K₂CO₃ (1.35 g, 9.79 mmol) and benzyltrimethylammonium tribromide (3.82 g, 9.79 mmol). The mixture was stirred at 40° C. for 5 h. The mixture was added into H₂O (100 mL) and the solid was filtered, washed with MeOH (3×20 mL) and dried under reduced pressure to provide a residue that was used directly. LCMS: m/z=302.9, 304.9, 306.9 [M+H]⁺.

methyl 2-(4,6-dibromo-1-oxophthalazin-2(1H)-yl)acetate: To a mixture of 4,6-dibromo-2H-phthalazin-1-one (13.0 g, 43.0 mmol) and methyl 2-bromoacetate (13.0 g, 86.0 mmol) in DMF (130 mL) was added Cs₂CO₃ (28.0 g, 85.5 mmol). The reaction mixture was stirred at 20° C. for 12 h. The reaction mixture was diluted with water (100 mL) and filtered. The solid was collected and dried under reduced pressure to provide a residue that was used directly. LCMS: m/z=374.8, 376.8, 378.8 [M+H]⁺.

### Intermediate 2

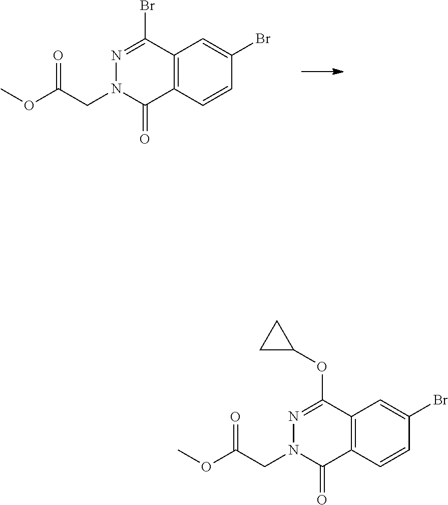 [SMILES: COC(=O)Cn1nc(Br)c2cc(Br)ccc2c1=O ; COC(=O)Cn1nc(OC2CC2)c2cc(Br)ccc2c1=O]

methyl 2-[6-bromo-4-(cyclopropoxy)-1-oxo-phthalazin-2-yl]acetate: To a solution of NaH (36 mg, 1.5 mmol) in DMF (2 mL) at 0° C. was added cyclopropanol (92 mg, 1.6 mmol). The mixture was stirred at 0° C. for 45 min. Methyl 2-(4,6-dibromo-1-oxo-phthalazin-2-yl)acetate (200 mg, 0.53 mmol) was added as a solid in one portion at 0° C. The mixture was stirred for 2 h at 0° C. The reaction mixture was diluted with aq. HCl (2 mL, 1 M) and extracted with EtOAc (2×10 mL). The combined organic layers were washed with brine (10 mL), dried over anhydrous Na₂SO₄, filtered, and concentrated under reduced pressure to provide a residue that was used directly. LCMS: m/z=353.2, 355.1 [M+H]⁺.

### Intermediate 3

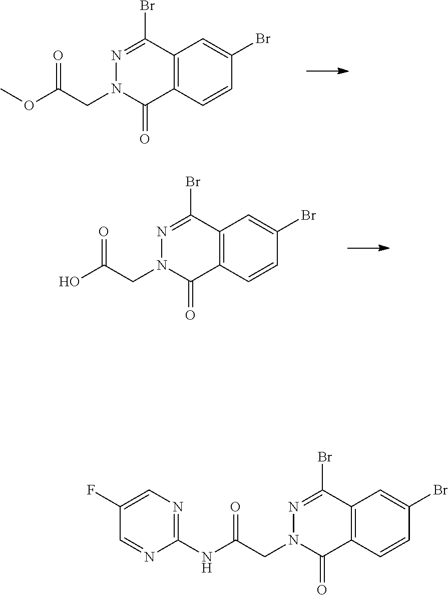 [SMILES: COC(=O)Cn1nc(Br)c2cc(Br)ccc2c1=O ; O=C(O)Cn1nc(Br)c2cc(Br)ccc2c1=O ; O=C(Cn1nc(Br)c2cc(Br)ccc2c1=O)Nc1ncc(F)cn1]

2-(4,6-dibromo-1-oxophthalazin-2(1H)-yl)acetic acid: To a solution of methyl 2-(4,6-dibromo-1-oxophthalazin-2(1H)-yl)acetate (5 g, 13.30 mmol) in THF (50 mL) and H₂O (10 mL) was added LiOH·H₂O (1.12 g, 26.6 mmol). The mixture was stirred for 2 h. The reaction mixture was poured into H₂O (50 mL) and the aqueous phase was acidified with aq. HCl (3M) to pH=2. The mixture was extracted with EtOAc (3×50 mL) and the combined organic layers were dried over anhydrous Na₂SO₄, filtered, and concentrated under reduced pressure. The residue was purified by reverse-phase preparative HPLC. LCMS: m/z=360.9, 362.9, 364.9 [M+H]⁺.

2-(4,6-dibromo-1-oxo-phthalazin-2-yl)-N-(5-fluoropyrimidin-2-yl)acetamide: To a solution of 2-(4,6-dibromo-1-oxo-phthalazin-2-yl)acetic acid (1.3 g, 3.6 mmol), 5-fluoropyrimidin-2-amine (736 mg, 6.5 mmol), and 1-methylimidazole (1.18 g, 14.4 mmol) in MeCN (13 mL) was added TCFH (1.4 g, 5.0 mmol). The reaction mixture was stirred at 40° C. for 2 h. A second portion of TCFH was added (1.4 g, 5.0 mmol) at 40° C. and the mixture was stirred for an additional 2 h. The reaction mixture was diluted with water (20 mL), filtered, and the filter cake was dried under reduced pressure to provide a residue that was used directly. LCMS: m/z=456.1, 458.1, 460.1 [M+H]⁺.

### Intermediate 4

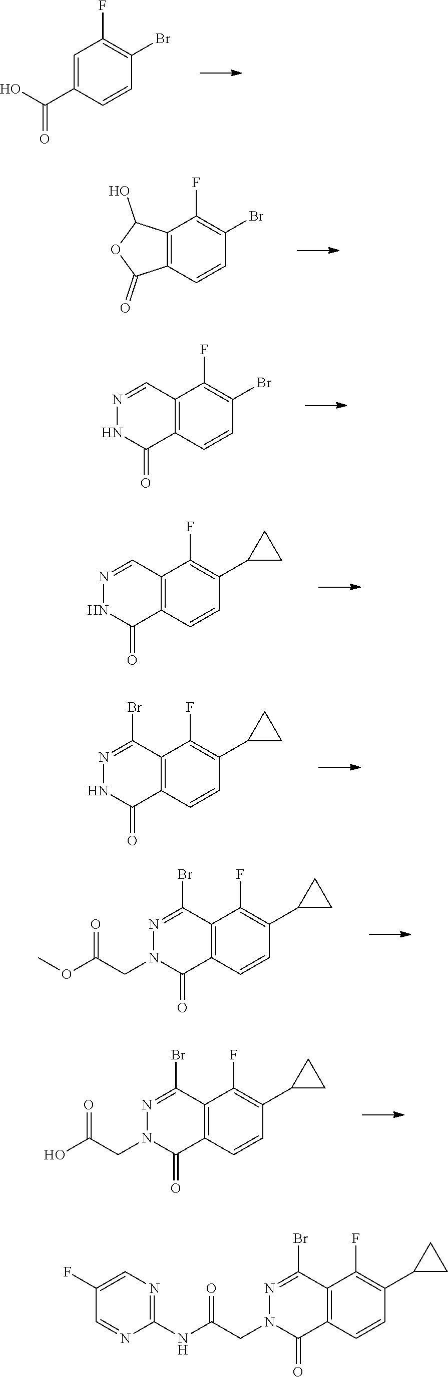

5-bromo-4-fluoro-3-hydroxyisobenzofuran-1(3H)-one: To a solution of TMP (80.6 g, 571 mmol) in THF (500 mL) at 0° C. was added n-BuLi (2.5 M in hexanes, 219 mL) dropwise. The reaction mixture was stirred at 0° C. for 0.5 h and then cooled to −45° C. To the reaction mixture was added a solution of 4-bromo-3-fluorobenzoic acid (50.0 g, 228 mmol) in THF (200 mL) dropwise. The reaction mixture was stirred at −45° C. for a further 5 h followed by the addition of DMF (25.0 g, 343 mmol). The reaction mixture was then stirred at 20° C. for a further 14.5 h. The reaction mixture was diluted with aq. HCl (3 M, 500 mL) and extracted with DCM (3×200 mL). The combined organics were washed with brine (400 mL), dried over anhydrous Na₂SO₄, filtered, and concentrated under reduced pressure. The crude residue was purified by silica gel column chromatography. ¹H NMR (400 MHz, CD₃CN): δ 7.89 (dd, J=6.0, 8.0 Hz, 1H), 7.55 (d, J=8.0 Hz, 1H), 6.72 (s, 1H), 5.99 (br s, 1H).

6-bromo-5-fluorophthalazin-1(2H)-one: To a solution of 5-bromo-4-fluoro-3-hydroxyisobenzofuran-1(3H)-one (2.0 g, 8.10 mmol) in THF (40 mL) was added NH₂NH₂·H₂O (405 mg, 8.10 mmol). The reaction mixture was stirred at 20° C. for 3 h. The reaction mixture was diluted with water (50 mL), filtered, and the filter cake was dried under reduced pressure to provide a residue that was used directly. LCMS: m/z=242.9, 244.9 [M+H]⁺.

6-cyclopropyl-5-fluorophthalazin-1(2H)-one: To a solution of 6-bromo-5-fluorophthalazin-1(2H)-one (5 g, 20.6 mmol) and potassium; cyclopropyl(trifluoro)boranuide (9.13 g, 61.7 mmol) in toluene (100 mL) and H₂O (10 mL) were added bis(1-adamantyl)-butyl-phosphane (1.48 g, 4.11 mmol), Pd(OAc)₂ (462 mg, 2.06 mmol), and Cs₂CO₃ (13.41 g, 41.2 mmol). The mixture was stirred at 105° C. for 3 h. The reaction mixture was quenched by addition of H₂O (200 mL) and extracted with EtOAc (3×70 mL). The combined organic layers were washed with brine (250 mL), dried over anhydrous Na₂SO₄, filtered and concentrated under reduced pressure to provide a crude residue that was used directly. LCMS: m/z=205.0 [M+H]⁺.

4-bromo-6-cyclopropyl-5-fluorophthalazin-1(2H)-one: To a solution of 6-cyclopropyl-5-fluorophthalazin-1(2H)-one (1.0 g, 4.90 mmol) in DMF (30 mL) at 0° C. were added K₂CO₃ (1.35 g, 9.79 mmol) and benzyltrimethylammonium tribromide (3.82 g, 9.79 mmol). The mixture was stirred at 40° C. for 5 h. The mixture was added into H₂O (100 mL) and the solid was filtered, washed with MeOH (3×20 mL) and dried under reduced pressure to provide a residue that was used directly. LCMS: m/z=283.0, 285.0 [M+H]⁺.

methyl 2-(4-bromo-6-cyclopropyl-5-fluoro-1-oxophthalazin-2(1H)-yl)acetate: To a solution of 4-bromo-6-cyclopropyl-5-fluorophthalazin-1(2H)-one (1.6 g, 5.65 mmol) in DMF (32 mL) at 0° C. were added Cs₂CO₃ (1.84 g, 5.65 mmol) and methyl 2-bromoacetate (865 mg, 5.65 mmol, 0.53 mL). The mixture was stirred at 20° C. for 1 h. The reaction mixture was diluted with H₂O (20 mL) and extracted with EtOAc (3×30 mL). The combined organic layers were washed with brine (30 mL), dried over anhydrous Na₂SO₄, filtered, and concentrated under reduced pressure. The residue was purified by silica gel column chromatography. LCMS: m/z=355.0, 357.0 [M+H]⁺.

### 2-(4-bromo-6-cyclopropyl-5-fluoro-1-oxophthalazin-2(1H)-yl)acetic acid

To a solution of methyl 2-(4-bromo-6-cyclopropyl-5-fluoro-1-oxophthalazin-2(1H)-yl)acetate (733 mg, 2.06 mmol) in THF (10 mL) and H₂O (10 mL) was added LiOH·H₂O (217 mg, 5.16 mmol). The mixture was stirred at 20° C. for 2 h. The reaction mixture was poured into H₂O (30 mL) and extracted with MTBE (3×20 mL). The aqueous phase was adjusted to pH=3 with aq. HCl (3 M) and extracted with EtOAc (3×30 mL). The combined organic layers were washed with brine (20 mL), dried over anhydrous Na₂SO₄, filtered and concentrated under reduced pressure to provide a residue that was used directly. LCMS: m/z=341.0, 343.0 [M+H]⁺.

2-(4-bromo-6-cyclopropyl-5-fluoro-1-oxophthalazin-2(1H)-yl)-N-(5-fluoropyrimidin-2-yl)acetamide: To a solution of 2-(4-bromo-6-cyclopropyl-5-fluoro-1-oxophthalazin-2(1H)-yl)acetic acid (550 mg, 1.61 mmol) and 5-fluoropyrimidin-2-amine (218 mg, 1.93 mmol) in pyridine (5 mL) was added 3-(ethyliminomethyleneamino)-N,N-dimethyl-propan-1-amine hydrochloride (463 mg, 2.42 mmol). The mixture was stirred for 16 h. The mixture was diluted with H₂O (10 mL). The solid was collected by filtration, washed with MTBE (5 mL×3) and dried under reduced pressure to provide a residue that was used directly. LCMS: m/z=436.0, 438.0 [M+H]⁺.

### Intermediate 5 and 6

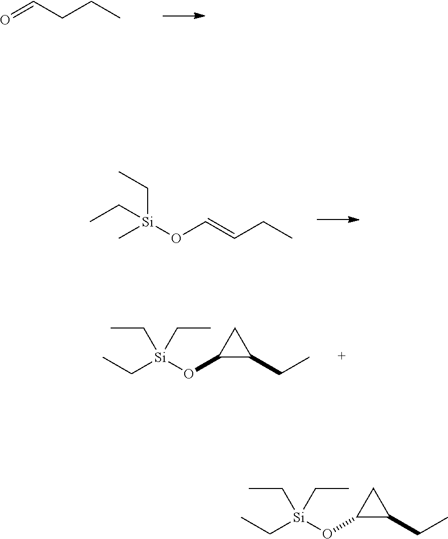

(but-1-en-1-yloxy)triethylsilane: To a solution of butyraldehyde (100 g, 1.39 mol) in DMF (1.0 L) were added Et₃N (336 g, 3.33 mol) and chloro(triethyl)silane (229 g, 1.53 mol) dropwise. The mixture was heated to 80° C. and stirred for 16 h. The mixture was cooled to ambient temperature, quenched with ice-cold H₂O (1.0 L), and extracted with DCM (3×500 mL). The combined organic layers were washed with brine (500 mL), dried over anhydrous Na₂SO₄, filtered, and concentrated under reduced pressure. The residue was purified by silica gel column chromatography.

cis-triethyl-2-ethylcyclopropoxysilane and trans-triethyl-2-ethylcyclopropoxysilane: To a solution of (but-1-en-1-yloxy)triethylsilane (50 g, 268 mmol) in DCM (250 mL) at 0° C. was added ZnEt₂ (1 M in toluene, 402 mL). The reaction mixture was stirred at 0° C. for 10 min prior to addition of CH₂I₂ (107 g, 402 mmol). The mixture was stirred at 20° C. for a further 16 h. The mixture was quenched with ice-cold H₂O (600 mL) and extracted with DCM (3×200 mL). The combined organic layers were washed with brine (200 mL), dried over anhydrous Na₂SO₄, filtered, and concentrated under reduced pressure. The residue was purified by silica gel column chromatography.

cis-triethyl-2-ethylcyclopropoxysilane (intermediate 5): ¹H NMR (400 MHz, CDCl₃) δ 3.12-3.02 (m, 1H), 1.40-1.23 (m, 2H), 1.01-0.93 (m, 12H), 0.90-0.78 (m, 1H), 0.68-0.59 (m, 8H).

trans-triethyl-2-ethylcyclopropoxysilane (intermediate 6): ¹H NMR (400 MHz, CDCl₃): δ 3.43-3.40 (m, 1H), 1.58-1.46 (m, 1H), 1.40-1.23 (m, 2H), 1.06-0.93 (m, 12H), 0.70-0.55 (m, 8H).

### Intermediate 7

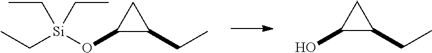

cis-2-ethylcyclopropanol: To a solution of cis-triethyl-2-ethylcyclopropoxysilane (4.0 g, 20 mmol) in 2-MeTHF (20 mL) at 0° C. was added aq. HCl (1 M, 24 mL). The mixture was stirred at 20° C. for 1 h. The mixture was diluted with H₂O (20 mL) and extracted with 2-MeTHF (3×10 mL). The combined organic layers were washed with brine (10 mL), dried over anhydrous Na₂SO₄, filtered, and concentrated under reduced pressure to give a residue that was used directly. ¹H NMR (400 MHz, CDCl₃): δ 3.55-3.51 (m, 1H), 1.96-1.86 (m, 1H), 1.51-1.47 (m, 2H), 1.25-1.23 (m, 1H), 0.67-0.64 (m, 2H), 0.65-0.57 (m, 2H).

### Intermediate 8

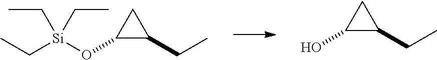

trans-2-ethylcyclopropanol: To a solution of trans-triethyl-2-ethylcyclopropoxysilane (2.0 g, 10 mmol) in 2-MeTHF (20 mL) at 0° C. was added aq. HCl (1 M, 12 mL). The mixture was stirred at 20° C. for 1 h. The mixture was diluted with H₂O (20 mL) and extracted with 2-MeTHF (3×10 mL). The combined organic layers were washed with brine (10 mL), dried over anhydrous Na₂SO₄, filtered, and concentrated under reduced pressure to give a residue that was used directly. ¹H NMR (400 MHz, CDCl₃): δ 3.08-3.05 (m, 1H), 1.34-1.05 (m, 2H), 0.82-0.62 (m, 1H), 0.62-0.40 (m, 5H).

### Intermediate 9 and 10

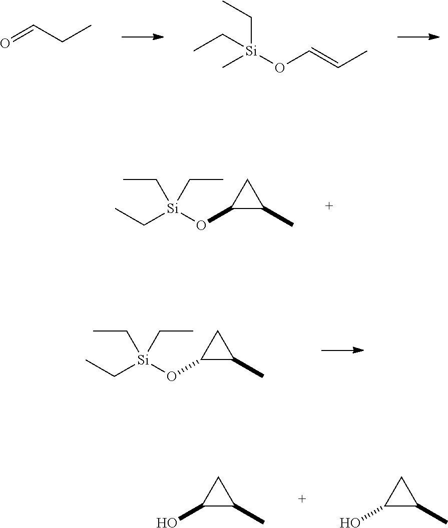

triethyl(prop-1-en-1-yloxy)silane: To a solution of chloro(triethyl)silane (81 g, 540 mmol) in DCM (300 mL) at 0° C. were added Et₃N (119 g, 1.18 mol) and propionaldehyde (30 g, 490 mmol, 95% purity). The reaction mixture was stirred at 20° C. for 16 h. The mixture was diluted with H₂O (300 mL) and extracted with DCM (3×100 mL). The combined organic layers were washed with brine (100 mL), dried over anhydrous Na₂SO₄, filtered, and concentrated under reduced pressure to provide a residue that was used directly.

triethyl(2-methylcyclopropoxy)silane: To a solution of triethyl(prop-1-en-1-yloxy)silane (41 g, 238 mmol) in DCM (410 mL) at 0° C. were added ZnEt₂ (1 M in toluene, 357 mL) followed by CH₂I₂ (96 g, 357 mmol). The mixture was stirred at 20° C. for 16 h. The mixture was quenched with H₂O (400 mL) and extracted with EtOAc (3×150 mL). The combined organic layers were washed with brine (150 mL), dried over anhydrous Na₂SO₄, filtered, and concentrated under reduced pressure to provide a residue that was used directly.

2-methylcyclopropanol: To a solution of triethyl(2-methylcyclopropoxy)silane (1.0 g, 5.37 mmol) in DMF (10 mL) was added CsF (4.08 g, 26.8 mmol). The mixture was stirred for 4 h. The mixture was filtered under reduced pressure to provide a residue that was used directly (1:1 mixture of isomers).

### Intermediate 11

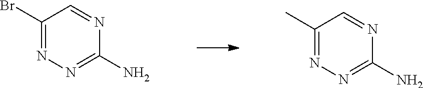

To a mixture of 6-bromo-1,2,4-triazin-3-amine (700 mg, 4.00 mmol) and Cs₂CO₃ (5.44 g, 16.7 mmol) in 1,4-dioxane (7.0 mL) were added 2,4,6-trimethyl-1,3,5,2,4,6-trioxatriborinane (3.59 g, 14.31 mmol, 50% purity) and Pd(dppf)Cl₂ (390 mg, 0.48 mmol). The mixture was stirred at 135° C. for 4 h in a sealed vial. The reaction mixture was cooled to ambient temperature, poured into H₂O (50 mL), and extracted with EtOAc (3×20 mL). The combined organic layers were dried over anhydrous Na₂SO₄, filtered, and concentrated under reduced pressure to give a residue. The residue was purified by reversed-phase preparative HPLC. ¹H NMR (400 MHz, CDCl₃): δ 8.08 (s, 1H), 5.25 (br s, 2H), 2.55 (s, 3H).

### Intermediate 12

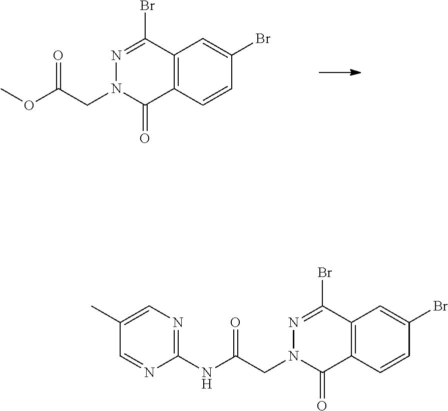 [SMILES: COC(=O)Cn1nc(Br)c2cc(Br)ccc2c1=O ; Cc1cnc(NC(=O)Cn2nc(Br)c3cc(Br)ccc3c2=O)nc1]

To a solution of methyl 2-(4,6-dibromo-1-oxo-phthalazin-2-yl)acetate (Intermediate 1, 500 mg, 1.33 mmol) and 5-methylpyrimidin-2-amine (290 mg, 2.66 mmol) in DCE (5.0 mL) was added AlMe₃ (1 M in n-heptane, 1.99 mL). The mixture was stirred at 90° C. for 3 h. The reaction mixture was poured into H₂O (15 mL) and extracted with DCM (3×20 mL). The combined organic layers were dried over anhydrous Na₂SO₄, filtered, and concentrated under reduced pressure to give a residue. The residue was purified by silica gel column. LCMS: m/z=451.9, 453.9, 455.9 [M+H]⁺.

### Intermediate 13

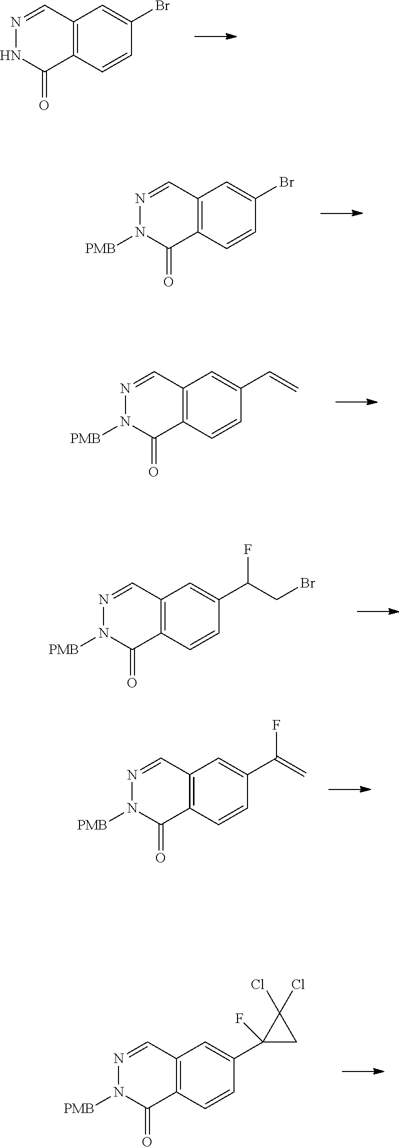

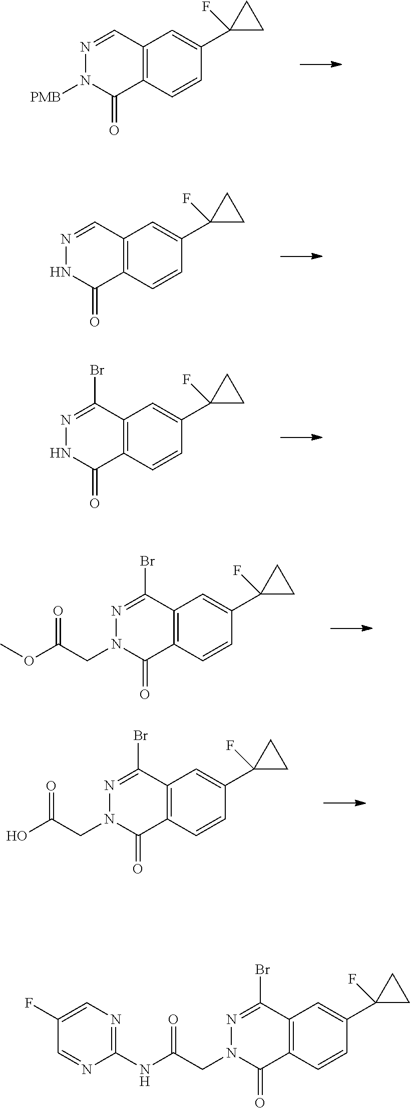

6-bromo-2-(4-methoxybenzyl)phthalazin-1(2H)-one: To a solution of 6-bromophthalazin-1(2H)-one (20.0 g, 88.9 mmol) in DMF (200 mL) were added Cs₂CO₃ (58.0 g, 178 mmol) and PMB-Cl (16.0 g, 102 mmol, 13.9 mL). The mixture was stirred for 16 h. The mixture was poured into H₂O (500 mL), filtered, and the filter cake was dried under reduced pressure to provide a residue that was used directly. LCMS: m/z=345.1, 347.1 [M+H]⁺.

2-(4-methoxybenzyl)-6-vinylphthalazin-1(2H)-one: To a solution of 6-bromo-2-(4-methoxybenzyl)phthalazin-1(2H)-one (11.5 g, 33.3 mmol) and potassium vinyltrifluoroborate (13.4 g, 100 mmol) in 1,4-dioxane (200 mL) were added CsF (15.0 g, 100 mmol) and Pd(dppf)Cl₂ (2.44 g, 3.33 mmol). The mixture was stirred at 100° C. for 12 h. The mixture was filtered, and the filtrate was diluted with H₂O (250 mL) and extracted with EtOAc (3×90 mL). The combined organic layers were washed with brine (150 mL), dried over anhydrous Na₂SO₄, filtered and concentrated under reduced pressure. The residue was purified by silica gel column chromatography. LCMS: m/z=293.1 [M+H]⁺.

6-(2-bromo-1-fluoroethyl)-2-(4-methoxybenzyl)phthalazin-1(2H)-one: To a solution of 2-(4-methoxybenzyl)-6-vinylphthalazin-1(2H)-one (10.0 g, 34 mmol) in sulfolane (100 mL) at 0° C. were added NBS (6.70 g, 37.6 mmol) and pyridine·HF (10.0 g, 102 mmol, 9.25 mL). The mixture was stirred at 20° C. for 12 h. The mixture was poured into H₂O (150 mL) and extracted with MTBE (3×50 mL). The combined organic layers were washed with brine (3×50 mL), dried over anhydrous Na₂SO₄, filtered and concentrated under reduced pressure. The residue was purified by silica gel column chromatography. LCMS: m/z=391.0, 393.0 [M+H]⁺.

6-(1-fluorovinyl)-2-(4-methoxybenzyl)phthalazin-1(2H)-one: To a solution of 6-(2-bromo-1-fluoroethyl)-2-(4-methoxybenzyl)phthalazin-1(2H)-one (3.9 g, 9.97 mmol) in DCM (40 mL) was added DBU (2.28 g, 14.9 mmol, 2.25 mL). The mixture was stirred at 45° C. for 16 h. The mixture was diluted with H₂O (60 mL) and extracted with DCM (3×20 mL). The combined organic layers were washed with brine (30 mL), dried over anhydrous Na₂SO₄, filtered and concentrated under reduced pressure. The residue was purified by silica gel column chromatography. LCMS: m/z=311.1 [M+H]⁺.

6-(2,2-dichloro-1-fluorocyclopropyl)-2-(4-methoxybenzyl)phthalazin-1(2H)-one: To a solution of 6-(1-fluorovinyl)-2-(4-methoxybenzyl)phthalazin-1(2H)-one (10.9 g, 35.1 mmol) and benzyltrimethylammonium chloride (320 mg, 1.40 mmol) in CHCl₃ (100 mL) at 0° C. was added NaOH (112.0 g, 1.40 mol, 100 mL, in H₂O) dropwise. The mixture was stirred at 0° C. for 0.5 h and then stirred at 20° C. for 12 h. The mixture was diluted with H₂O (25 mL) and extracted with DCM (3×10 mL). The combined organic layers were washed with brine (15 mL), dried over anhydrous Na₂SO₄, filtered and concentrated under reduced pressure. The residue was purified by silica gel column chromatography. LCMS: m/z=393.1, 395.0, 397.0 [M+H]⁺.

6-(1-fluorocyclopropyl)-2-(4-methoxybenzyl)phthalazin-1(2H)-one: A mixture of 6-(2,2-dichloro-1-fluorocyclopropyl)-2-(4-methoxybenzyl)phthalazin-1(2H)-one (100 mg, 0.25 mmol) and AIBN (4.0 mg, 0.03 mmol) in tributyltin hydride (770 mg, 2.65 mmol, 0.7 mL) was stirred at 160° C. for 3 h. The mixture was concentrated under reduced pressure. The residue was purified by silica gel column chromatography. LCMS: m/z=325.1 [M+H]⁺.

6-(1-fluorocyclopropyl)phthalazin-1(2H)-one: To a solution of 6-(1-fluorocyclopropyl)-2-(4-methoxybenzyl)phthalazin-1(2H)-one (3.2 g, 9.87 mmol) in TFA (35 mL) was added TfOH (5.44 g, 36.3 mmol, 3.20 mL). The mixture was stirred at 60° C. for 12 h. The mixture was poured into H₂O (30 mL) and sat. aq. NaHCO₃ (30 mL) and extracted with DCM (3×20 mL). The combined organic layers were washed with brine (50 mL), dried over anhydrous Na₂SO₄, filtered and concentrated under reduced pressure. The residue was purified by silica gel column chromatography. LCMS: m/z=205.1 [M+H]⁺.

4-bromo-6-(1-fluorocyclopropyl)phthalazin-1(2H)-one: To a solution of 6-(1-fluorocyclopropyl)phthalazin-1(2H)-one (1.0 g, 4.90 mmol) in DMF (30 mL) were added K₂CO₃ (1.35 g, 9.79 mmol) and benzyltrimethylammonium tribromide (3.82 g, 9.79 mmol). The mixture was stirred at 40° C. for 2 h. The mixture was poured into H₂O (40 mL) and extracted with EtOAc (3×15 mL). The combined organic layers were washed with brine (20 mL), dried over anhydrous Na₂SO₄, filtered and concentrated under reduced pressure to give a residue that was used directly. LCMS: m/z=282.9, 284.9 [M+H]⁺.

methyl 2-(4-bromo-6-(1-fluorocyclopropyl)-1-oxophthalazin-2(1H)-yl)acetate: To a solution of 4-bromo-6-(1-fluorocyclopropyl)phthalazin-1(2H)-one (600 mg, 2.12 mmol) in DMF (10 mL) were added Cs₂CO₃ (1.38 g, 4.24 mmol) and methyl 2-iodoacetate (424 mg, 2.12 mmol). The mixture was stirred for 2 h. The mixture was diluted with H₂O (15 mL) and extracted with EtOAc (3×5 mL). The combined organic layers were washed with brine (10 mL), dried over anhydrous Na₂SO₄, filtered and concentrated under reduced pressure. The residue was purified by silica gel column chromatography. LCMS: m/z=355.0, 357.0 [M+H]⁺.

2-(4-bromo-6-(1-fluorocyclopropyl)-1-oxophthalazin-2(1H)-yl)acetic acid: To a solution of methyl 2-(4-bromo-6-(1-fluorocyclopropyl)-1-oxophthalazin-2(1H)-yl)acetate (200 mg, 0.56 mmol) in THF (2.0 mL) and H₂O (2.0 mL) was added LiOH·H₂O (59 mg, 1.41 mmol). The mixture was stirred for 1 h. The mixture was poured into H₂O (5 mL) and extracted with EtOAc (3×2 mL). The combined organic layers were washed with brine (5 mL), dried over anhydrous Na₂SO₄, filtered and concentrated under reduced pressure to provide a residue that was used directly. LCMS: m/z=341.0, 343.0 [M+H]⁺.

2-(4-bromo-6-(1-fluorocyclopropyl)-1-oxophthalazin-2(1H)-yl)-N-(5-fluoropyrimidin-2-yl)acetamide: To a solution of 2-(4-bromo-6-(1-fluorocyclopropyl)-1-oxophthalazin-2(1H)-yl)acetic acid (128 mg, 0.37 mmol) in pyridine (2 mL) was added 5-fluoropyrimidin-2-amine (63 mg, 0.56 mmol) and EDCI (143 mg, 0.75 mmol). The mixture was stirred for 3 h. The mixture was poured into H₂O (5 mL) and extracted with EtOAc (3×2 mL). The combined organic layers were washed with brine (5 mL), dried over anhydrous Na₂SO₄, filtered and concentrated under reduced pressure. The residue was suspended in DCM (3 mL) and i-PrOH (0.5 mL). The mixture was stirred for 10 min. The mixture was filtered and the filter cake was dried under reduced pressure to provide a residue that was used directly. LCMS: m/z=436.0, 438.0 [M+H]⁺.

### Intermediate 14

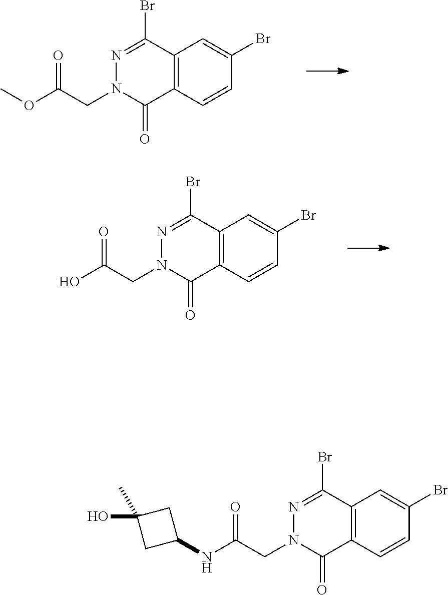

2-(4,6-dibromo-1-oxophthalazin-2(1H)-yl)acetic acid: To a solution of methyl 2-(4,6-dibromo-1-oxophthalazin-2(1H)-yl)acetate (5.0 g, 13.3 mmol) in THF (50 mL) and H₂O (10 mL) was added LiOH·H₂O (1.12 g, 26.6 mmol). The reaction mixture was stirred for 2 h. The reaction mixture was poured into H₂O (50 mL) and the aqueous phase was acidified with aq. HCl (3M) to pH=2. The mixture was extracted with EtOAc (3×50 mL) and the combined organic layers were dried over anhydrous Na₂SO₄, filtered, and concentrated under reduced pressure. The residue was purified by reverse-phase preparative HPLC. LCMS: m/z=360.9, 362.9, 364.9 [M+H]⁺.

2-(4,6-dibromo-1-oxophthalazin-2(1H)-yl)-N-(cis-3-hydroxy-3-methylcyclobutyl)acetamide: To a solution of 2-(4,6-dibromo-1-oxophthalazin-2(1H)-yl)acetic acid (300 mg, 0.83 mmol) in DMF (4 mL) at 0° C. were added cis-3-amino-1-methylcyclobutanol HCl salt (125 mg, 0.91 mmol), DIPEA (530 mg, 4.14 mmol), HOBt (168 mg, 1.24 mmol), and EDCI (239 mg, 1.24 mmol). The mixture was stirred at 20° C. for 12 h. The mixture was poured into H₂O (5 mL) and EtOAc (3 mL), filtered, and the filter cake was dried under reduce pressure. The residue was triturated with EtOAc (3 mL), dried under reduced pressure, and used directly. LCMS: m/z=443.9, 446.0, 447.9 [M+H]⁺.

### Intermediate 15 and 16

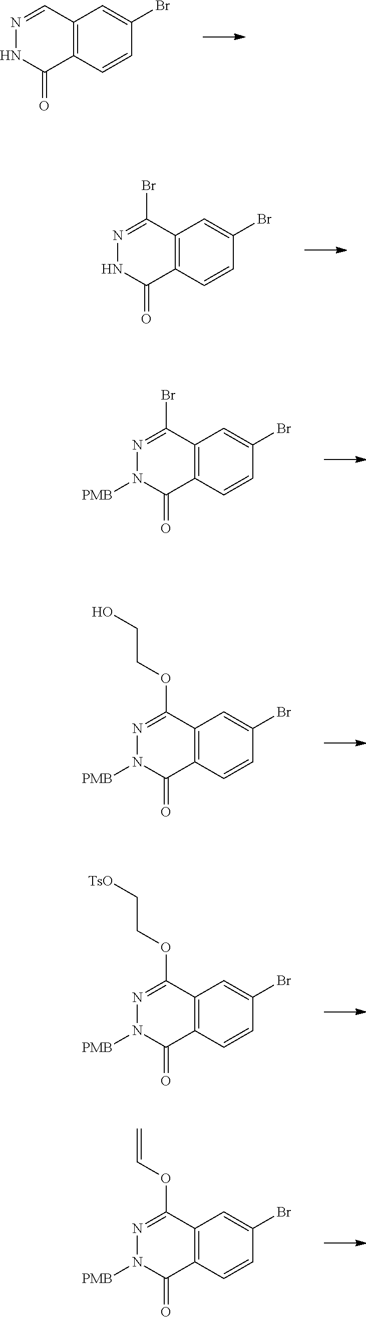

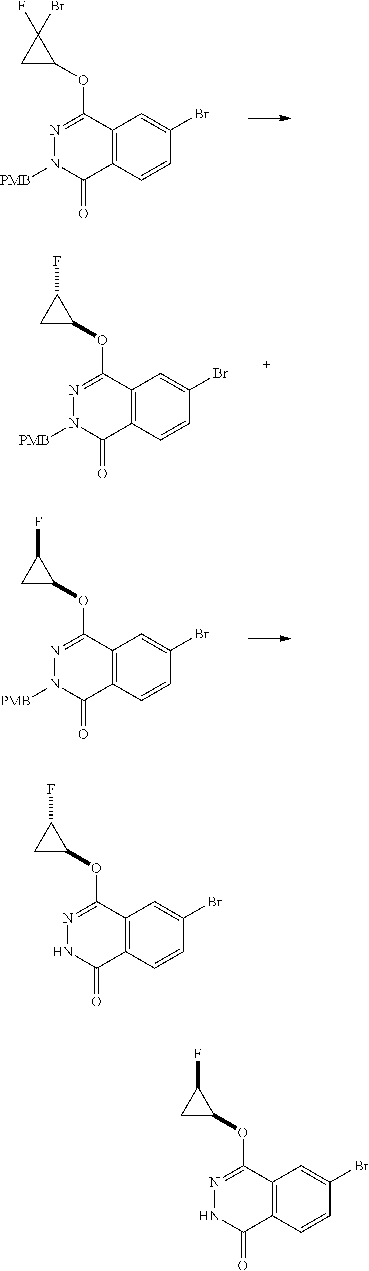

4,6-dibromophthalazin-1(2H)-one: To a mixture of 6-bromophthalazin-1(2H)-one (50 g, 222 mmol) in DMF (500 mL) were added K₂CO₃ (61.4 g, 444 mmol) and benzyltrimethylammonium tribromide (173 g, 444 mmol). The mixture was stirred at 40° C. for 3 h. The mixture was poured into ice-cold H₂O (200 mL) and filtered. The filter cake was dried under reduced pressure to provide a residue that was used directly. LCMS: m/z=302.9, 304.9, 306.9 [M+H]⁺.

4,6-dibromo-2-(4-methoxybenzyl)phthalazin-1(2H)-one: To a mixture of 4,6-dibromophthalazin-1(2H)-one (64 g, 211 mmol) in DMF (100 mL) were added Cs₂CO₃ (137 g, 421 mmol) and PMB-Cl (37.9 g, 242 mmol). The mixture was stirred for 12 h. The mixture was poured into ice-cold H₂O (200 mL), filtered, and the filter cake was dried under reduced pressure to provide a residue that was used directly. LCMS: m/z=423.0, 424.8, 426.9 [M+H]⁺.

6-bromo-4-(2-hydroxyethoxy)-2-(4-methoxybenzyl)phthalazin-1(2H)-one: To a mixture of ethylene glycol (40.9 g, 660 mmol) in DMF (600 mL) at 0° C. was added NaH (10.6 g, 264 mmol, 60% purity). The mixture was stirred for 30 min at 0° C. To the mixture was added a solution of 4,6-dibromo-2-(4-methoxybenzyl)phthalazin-1(2H)-one (56 g, 132 mmol) in DMF (600 mL). The reaction mixture was warmed to 25° C. and stirred for 12 h. The mixture was poured into ice-cold H₂O, filtered, and the filter cake was dried under reduced pressure to provide a residue that was used directly. LCMS: m/z=405.0, 407.0 [M+H]⁺.

2-((7-bromo-3-(4-methoxybenzyl)-4-oxo-3,4-dihydrophthalazin-1-yl)oxy)ethyl 4-methylbenzenesulfonate: To a mixture of 6-bromo-4-(2-hydroxyethoxy)-2-(4-methoxybenzyl)phthalazin-1(2H)-one (42 g, 104 mmol) and Et₃N (20.9 g, 207 mmol) in DCM (500 mL) was added 4-methylbenzenesulfonyl chloride (21.7 g, 114 mmol). The mixture was stirred for 12 h. The mixture was poured into ice-cold H₂O (30 mL) and extracted with EtOAc (3×15 mL). The combined organic layers were washed with brine (20 mL), dried over anhydrous Na₂SO₄, filtered, and concentrated under reduced pressure to provide a residue that was used directly. LCMS: m/z=559.0, 561.0 [M+H]⁺.

6-bromo-2-(4-methoxybenzyl)-4-(vinyloxy)phthalazin-1(2H)-one: To a mixture of 2-((7-bromo-3-(4-methoxybenzyl)-4-oxo-3,4-dihydrophthalazin-1-yl)oxy)ethyl 4-methylbenzenesulfonate (40.9 g, 73.1 mmol) in DMSO (400 mL) was added t-BuOK (10.7 g, 95.1 mmol). The mixture was stirred for 1 h. The mixture was poured into aq. sat. NH₄Cl (800 mL), filtered, and the filter cake was dried under reduced pressure. The residue was purified by silica gel column chromatography. LCMS: m/z=387.0, 389.0 [M+H]⁺.

6-bromo-4-(2-bromo-2-fluorocyclopropoxy)-2-(4-methoxybenzyl)phthalazin-1(2H)-one: To a solution of 6-bromo-2-(4-methoxybenzyl)-4-(vinyloxy)phthalazin-1(2H)-one (3.0 g, 7.75 mmol) in dibromo(fluoro)methane (74.3 g, 387 mmol) was added benzyltriethylammonium chloride (176 mg, 0.77 mmol). To the mixture at 0° C. was added aq. NaOH (31 g, 387 mmol, 50 wt %). The mixture was stirred at 20° C. for 16 h. The mixture was diluted with H₂O (15 mL) and extracted with EtOAc (3×20 mL). The combined organic layers were washed with brine (10 mL), dried over anhydrous Na₂SO₄, filtered and concentrated under reduced pressure to provide a residue that was used directly. LCMS: m/z=497.0, 499.0, 500.9 [M+H]⁺.

6-bromo-4-(2-fluorocyclopropoxy)-2-(4-methoxybenzyl)phthalazin-1(2H)-one: To a solution 6-bromo-4-(2-bromo-2-fluorocyclopropoxy)-2-(4-methoxybenzyl)phthalazin-1(2H)-one (900 mg, 1.08 mmol) in EtOH (9 mL) and DCE (9 mL) was added Zn (709 mg, 10.8 mmol) and NH₄Cl (1.93 g, 36.1 mmol). The mixture was stirred at 80° C. for 2 h. The mixture was filtered and the filtrate was concentrated under reduced pressure to provide a residue that was used directly. LCMS: m/z=419.0, 421.0 [M+H]⁺.

6-bromo-4-(2-trans-fluorocyclopropoxy)phthalazin-1(2H)-one and 6-bromo-4-((2-cis-fluorocyclopropoxy)phthalazin-1(2H)-one: To a solution of 6-bromo-4-(2-fluorocyclopropoxy)-2-(4-methoxybenzyl)phthalazin-1(2H)-one (1.2 g) in DCM (12 mL) were added TFA (12.0 mL, 162 mmol) and TfOH (4.08 g, 27.1 mmol, 2.40 mL). The mixture was stirred at 20° C. for 2 h. The reaction was poured into H₂O (20 mL) and extracted with DCM (3×10 mL). The combined organic layers were washed with brine (10 mL), dried over anhydrous Na₂SO₄, filtered and concentrated under reduced pressure. The residue was purified by silica gel column chromatography.

6-bromo-4-(2-trans-fluorocyclopropoxy)phthalazin-1(2H)-one: LCMS: m/z=298.9, 300.9 [M+H]⁺. ¹H NMR (400 MHz, CDCl₃): δ 9.59 (br s, 1H), 8.28-8.25 (m, 1H), 8.02-8.01 (m, 1H), 7.93-7.91 (m, 1H), 4.87-4.71 (m, 1H), 4.52-4.46 (m, 1H), 2.01-1.87 (m, 1H), 1.59-1.51 (m, 1H).

6-bromo-4-(2-cis-fluorocyclopropoxy)phthalazin-1(2H)-one: LCMS: m/z=298.9, 300.9 [M+H]⁺. ¹H NMR (400 MHz, CDCl₃): δ 9.84 (br s, 1H), 8.27 (d, J=8.0 Hz, 1H), 8.14 (d, J=1.2 Hz, 1H), 7.92 (dd, J=6.4, 2.0 Hz, 1H), 4.80-4.62 (m, 1H), 4.10-4.06 (m, 1H), 1.41-1.25 (m, 2H).

### Example 1

### 2-[6-bromo-4-(2,2-difluorospiro[3.3]heptan-6-yl)oxy-1-oxophthalazin-2-yl]-N-(5-fluoropyrimidin-2-yl)acetamide

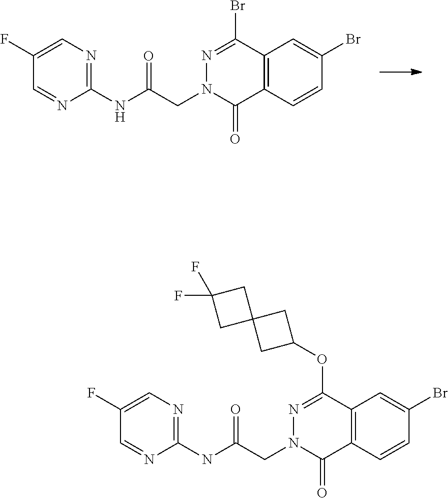

To a solution of 6,6-difluorospiro[3.3]heptan-2-ol (48 mg, 0.32 mmol) in THF (1.0 mL) at 0° C. was added NaH (8 mg, 0.21 mmol, 60% purity). The mixture was stirred at 0° C. for 0.5 h and then warmed to 20° C. To the mixture was added 2-(4,6-dibromo-1-oxophthalazin-2(1H)-yl)-N-(5-fluoropyrimidin-2-yl)acetamide (50 mg, 0.11 mmol). The reaction mixture was stirred at 80° C. for a further 2 h. The mixture was poured into H₂O (3 mL) and extracted with EtOAc (3×1 mL). The combined organic layers were washed with brine (3 mL), dried over anhydrous Na₂SO₄, filtered, and concentrated under reduced pressure. The residue was purified by reverse-phase preparative HPLC. LCMS: m/z=524.0, 526.0 [M+H]⁺. ¹H NMR (400 MHz, CDCl₃): δ 8.87 (br s, 1H), 8.49 (s, 2H), 8.29 (d, J=8.4 Hz, 1H), 8.13 (d, J=2.0 Hz, 1H), 7.91 (dd, J=2.0, 8.4 Hz, 1H), 5.21 (br s, 2H), 5.12-5.05 (m, 1H), 2.73-2.68 (m, 2H), 2.67 (br s, 2H), 2.65-2.59 (m, 2H), 2.44-2.35 (m, 2H).

### Example 2 and 3

### 2-[6-bromo-4-[2-trans-methylcyclopropyl]oxy-1-oxophthalazin-2-yl]-N-(5-fluoropyrimidin-2-yl)acetamide

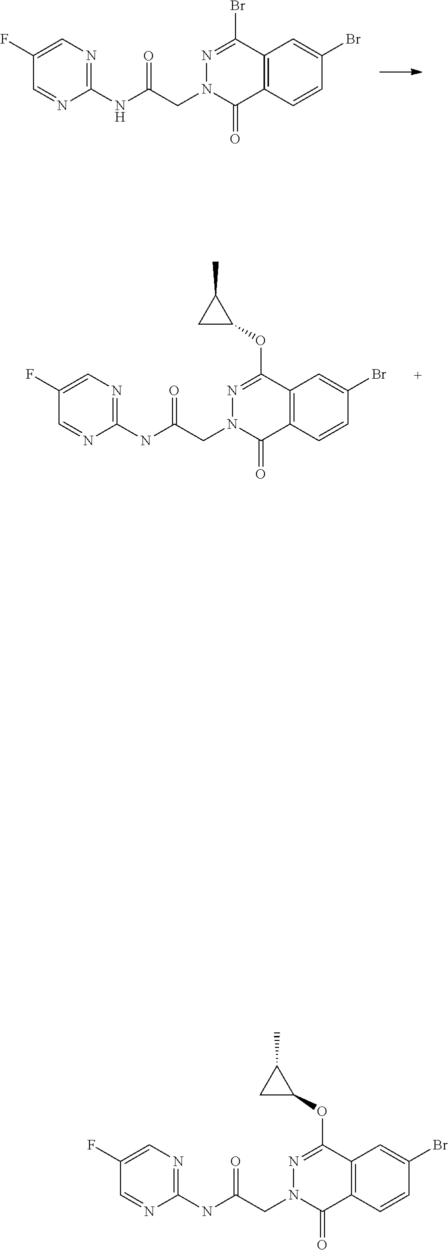

To a solution of NaH (12 mg, 0.52 mmol) in DMF (0.5 mL) at 0° C. was added 2-methylcyclopropanol (28 mg, 0.39 mmol, 1:1 mixture of isomers). The reaction mixture was stirred at 0° C. for 45 min followed by the addition of 2-(4,6-dibromo-1-oxophthalazin-2(1H)-yl)-N-(5-fluoropyrimidin-2-yl)acetamide (60 mg, 0.13 mmol). The reaction mixture was stirred for 0° C. for 2 h. The reaction mixture was diluted with aq. HCl (1 M, 1 mL) and extracted with EtOAc (2×10 mL). The combined organic layers were washed with brine (5 mL), dried over anhydrous Na₂SO₄, filtered, and concentrated under reduced pressure. The residue was purified by reverse-phase preparative HPLC and further purified by chiral SFC (Column: Daicel ChiralPak AD (250 mm×30 mm×10 m); Mobile phase: A: CO₂, B: EtOH; Gradient: 55% B isocratic; Flow rate: 80 g/min; Detection Wavelength: 220 nm; Column temperature: 40° C.; System Back Pressure: 100 bar) to provide:

2-[6-bromo-4-[2-trans-methylcyclopropyl]oxy-1-oxophthalazin-2-yl]-N-(5-fluoropyrimidin-2-yl)acetamide (first eluting isomer, 2). LCMS: m/z=447.9, 449.9 [M+H]⁺. ¹H NMR (400 MHz, CDCl₃): δ 9.11 (br s, 1H), 8.49 (s, 2H), 8.28 (d, J=8.4 Hz, 1H), 8.06 (d, J=1.8 Hz, 1H), 7.89 (dd, J=2.0, 8.8 Hz, 1H), 5.25 (br s, 2H), 3.94-3.83 (m, 1H), 1.21-1.13 (m, 1H), 1.13-1.08 (m, 3H), 1.04-0.91 (m, 1H), 0.64-0.60 (m, 1H); and

2-[6-bromo-4-[2-trans-methylcyclopropyl]oxy-1-oxophthalazin-2-yl]-N-(5-fluoropyrimidin-2-yl)acetamide (second eluting isomer, 3). LCMS: m/z=447.9, 449.9 [M+H]⁺. ¹H NMR (400 MHz, CDCl₃): δ 9.11 (br s, 1H), 8.49 (s, 2H), 8.28 (d, J=8.4 Hz, 1H), 8.06 (d, J=1.8 Hz, 1H), 7.89 (dd, J=2.0, 8.8 Hz, 1H), 5.25 (br s, 2H), 3.94-3.83 (m, 1H), 1.21-1.13 (m, 1H), 1.13-1.08 (m, 3H), 1.04-0.91 (m, 1H), 0.64-0.60 (m, 1H).

### Example 4

### 2-(6-bromo-1-oxo-4-spiro[2.3]hexan-5-yloxyphthalazin-2-yl)-N-(5-fluoropyrimidin-2-yl)acetamide

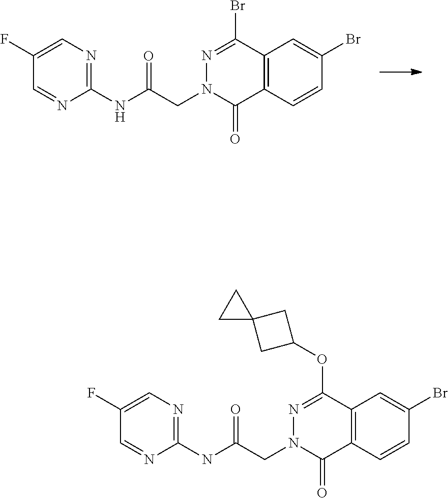

To a solution of NaH (26 mg, 0.66 mmol, 60% purity) in DMF (1.0 mL) at 0° C. was added spiro[2.3]hexan-5-ol (23 mg, 0.22 mmol). The mixture was stirred at 0° C. for 45 min. To the mixture was added 2-(4,6-dibromo-1-oxophthalazin-2(1H)-yl)-N-(5-fluoropyrimidin-2-yl)acetamide (50 mg, 0.11 mmol) as a solid in one portion and the mixture was stirred for 2 h at 0° C. The reaction mixture was poured into H₂O (15 mL) and then extracted with EtOAc (3×15 mL). The combined organic layers were dried over anhydrous Na₂SO₄, filtered, and concentrated under reduced pressure. The residue was purified by reverse-phase preparative HPLC. LCMS: m/z=474.2, 476.2 [M+H]⁺. ¹H NMR (400 MHz, DMSO-d₆): δ 11.09 (s, 1H), 8.78 (s, 2H), 8.18-8.15 (m, 2H), 8.11 (dd, J=8.5, 1.9 Hz, 1H), 5.28-5.21 (m, 1H), 4.98 (s, 2H), 2.47-2.43 (m, 4H), 0.51-0.43 (m, 4H).

### Example 5 and 6

### 2-[6-bromo-4-(cis-2,2-difluorospiro[2.3]hexan-5-yl)oxy-1-oxophthalazin-2-yl]-N-(5-fluoropyrimidin-2-yl)acetamide and 2-[6-bromo-4-(trans-2,2-difluorospiro[2.3]hexan-5-yl)oxy-1-oxophthalazin-2-yl]-N-(5-fluoropyrimidin-2-yl)acetamide

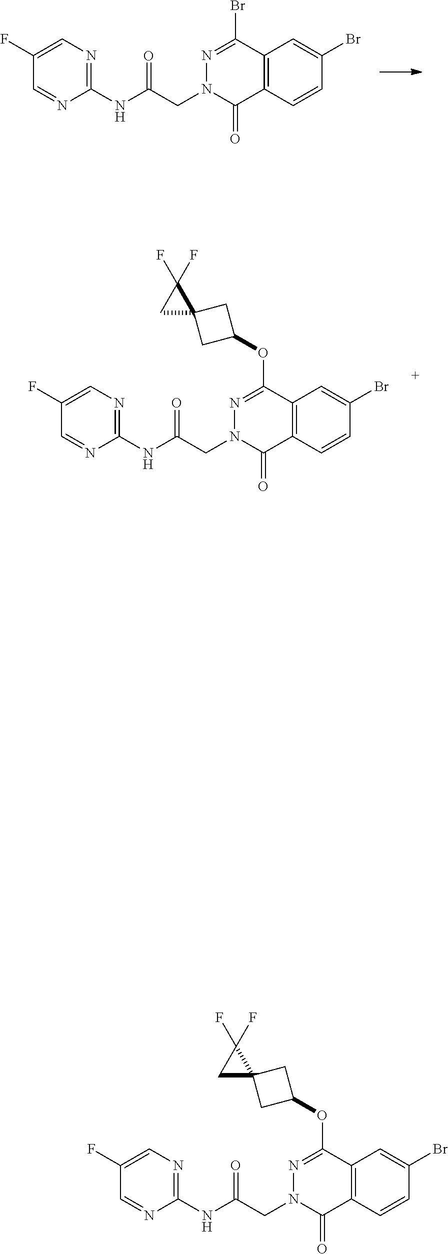

To a solution of 1,1-difluorospiro[2.3]hexan-5-ol (73 mg, 0.55 mmol) in THF (1.0 mL) at 0° C. was added NaH (9 mg, 0.22 mmol, 60% purity). The mixture was stirred at 0° C. for 30 min. To the mixture was added 2-(4,6-dibromo-1-oxophthalazin-2(1H)-yl)-N-(5-fluoropyrimidin-2-yl)acetamide (50 mg, 0.11 mmol) and the mixture was stirred at 80° C. for 2 h. The reaction mixture was diluted with H₂O (5 mL) and extracted with EtOAc (3×5 mL). The combined organic layers were washed with brine (5 mL), dried over anhydrous Na₂SO₄, filtered, and concentrated under reduced pressure. The residue was purified by reverse-phase preparative HPLC and further purified by SFC (Column: Daicel Chiralpak AD (250 mm×30 mm, 10 μm particle size); Mobile phase: A: CO₂ B: 0.1% NH₄OH in i-PrOH; Gradient: B %=50% isocratic; Detection wavelength: 220 nm; Flow rate: 4 mL/min; Column temperature: 35° C.; System back pressure: 124 bar) to provide:

2-[6-bromo-4-(2,2-difluorospiro[2.3]hexan-5-yl)oxy-1-oxophthalazin-2-yl]-N-(5-fluoropyrimidin-2-yl)acetamide (first eluting isomer, 5): LCMS: m/z=510.0, 512.0 [M+H]⁺. ¹H NMR (400 MHz, CDCl₃): δ 8.99 (br s, 1H), 8.48 (s, 2H), 8.30 (d, J=8.4 Hz, 1H), 8.18 (d, J=2.0 Hz, 1H), 7.93 (dd, J=2.0, 8.8 Hz, 1H), 5.40-5.32 (m, 1H), 5.30-5.07 (m, 2H), 2.72-2.64 (m, 2H), 2.63-2.52 (m, 2H), 1.33 (t, J=8.2 Hz, 2H).

2-[6-bromo-4-(2,2-difluorospiro[2.3]hexan-5-yl)oxy-1-oxophthalazin-2-yl]-N-(5-fluoropyrimidin-2-yl)acetamide (second eluting isomer, 6): LCMS: m/z=510.0, 512.0 [M+H]⁺. ¹H NMR (400 MHz, CDCl₃): δ 8.90 (br s, 1H), 8.51 (s, 2H), 8.31 (d, J=8.4 Hz, 1H), 8.18 (d, J=1.6 Hz, 1H), 7.93 (dd, J=2.0, 8.8 Hz, 1H), 5.34-5.18 (m, 3H), 2.86-2.76 (m, 2H), 2.55-2.44 (m, 2H), 1.32 (t, J=8.4 Hz, 2H).

### Example 7 and 8

### 2-[6-bromo-4-[trans-2-methylcyclopropyl]oxy-1-oxophthalazin-2-yl]-N-(cis-3-hydroxy-3-methylcyclobutyl)acetamide

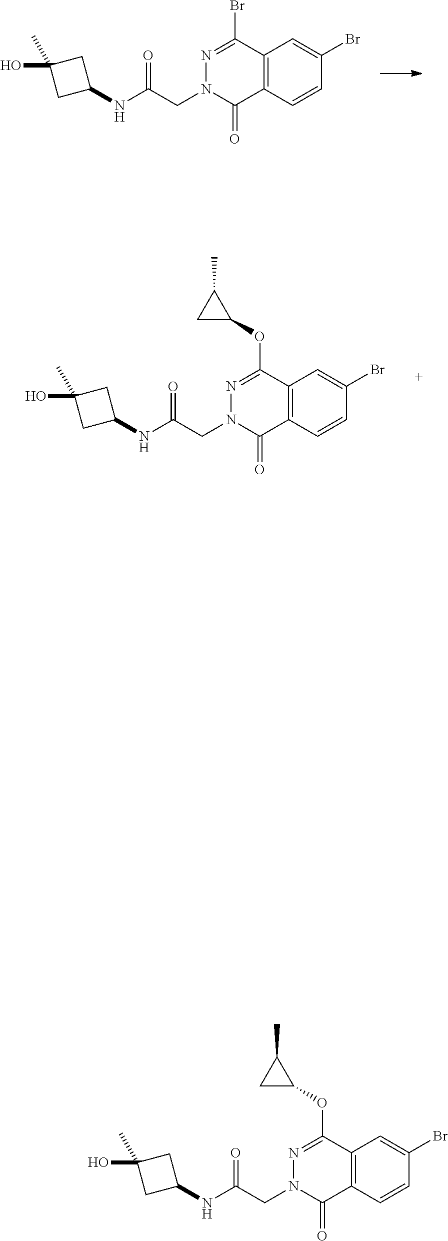 [SMILES: C[C@]1(O)C[C@@H](NC(=O)Cn2nc(Br)c3cc(Br)ccc3c2=O)C1 ; C[C@H]1C[C@@H]1Oc1nn(CC(=O)N[C@H]2C[C@@](C)(O)C2)c(=O)c2ccc(Br)cc12 ; C[C@@H]1C[C@H]1Oc1nn(CC(=O)N[C@H]2C[C@@](C)(O)C2)c(=O)c2ccc(Br)cc12]

To a solution of NaH (554 mg, 13.9 mmol, 60% purity) in DMF (5 mL) at 0° C. was added 2-methylcyclopropanol (500 mg, 6.93 mmol, 1:1 mixture of isomers). The mixture was stirred for 30 min at 0° C. To the mixture was added 2-(4,6-dibromo-1-oxophthalazin-2(1H)-yl)-N-(cis-3-hydroxy-3-methylcyclobutyl)acetamide (247 mg, 0.55 mmol) and the mixture was stirred at 20° C. for 2 h. The mixture was poured into aq. sat. NH₄Cl (10 mL) and extracted with EtOAc (3×10 mL). The combined organic layers were dried over anhydrous Na₂SO₄, filtered, and concentrated under reduced pressure. The residue was purified by reverse-phase preparative HPLC and further purified by chiral SFC (Column: Daicel Chiralpak AD (250 mm×30 mm, 10 μm particle size); Mobile phase: A: CO₂ B: 0.1% NH₃—H₂O in i-PrOH; Gradient: B %=23% isocratic; Detection wavelength: 220 nm; Flow rate: 2.5 mL/min; Column temperature: 35° C.; System back pressure: 137 bar) to provide:

2-[6-bromo-4-[trans-2-methylcyclopropyl]oxy-1-oxophthalazin-2-yl]-N-(cis-3-hydroxy-3-methylcyclobutyl)acetamide (first eluting isomer, 7): LCMS: m/z=436.0, 438.0 [M+H]⁺. ¹H NMR (400 MHz, DMSO-d₆): δ 8.20 (d, J=7.2 Hz, 1H), 8.16-8.12 (m, 1H), 8.09-8.05 (m, 1H), 8.00 (d, J=2.0 Hz, 1H), 4.95 (s, 1H), 4.70-4.43 (m, 2H), 3.88-3.78 (m, 2H), 2.26-2.16 (m, 2H), 2.00-1.91 (m, 2H), 1.21 (s, 3H), 1.14-1.08 (m, 1H), 1.07-1.03 (m, 3H), 1.02-0.96 (m, 1H), 0.63-0.60 (m, 1H).

2-[6-bromo-4-[trans-2-methylcyclopropyl]oxy-1-oxophthalazin-2-yl]-N-(cis-3-hydroxy-3-methylcyclobutyl)acetamide (second eluting isomer, 8): LCMS: m/z=436.0, 438.0 [M+H]⁺. ¹H NMR (400 MHz, DMSO-d₆): δ 8.22 (d, J=7.2 Hz, 1H), 8.18-8.13 (m, 1H), 8.11-8.07 (m, 1H), 8.04 (d, J=2.0 Hz, 1H), 4.95 (s, 1H), 4.68-4.43 (m, 2H), 4.17-4.13 (m, 1H), 3.85-3.69 (m, 1H), 2.27-2.17 (m, 2H), 1.94 (br t, J=9.2 Hz, 2H), 1.21 (s, 3H), 1.08-1.05 (m, 4H), 0.99-0.92 (m, 1H), 0.55-0.48 (m, 1H).

### Example 9 and 10

### 2-[6-bromo-4-[trans-2-methylcyclopropyl]oxy-1-oxophthalazin-2-yl]-N-(5-methylpyrimidin-2-yl)acetamide

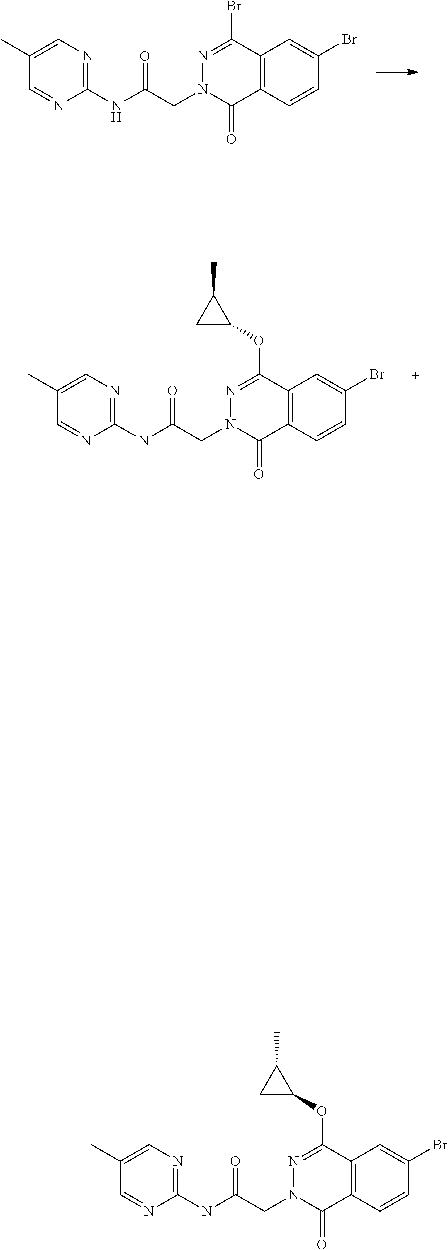

To a solution of NaH (35 mg, 0.88 mmol, 60% purity) in DMF (5 mL) at 0° C. was added 2-methylcyclopropanol (493 mg, 6.84 mmol, 1:1 mixture of isomers). The mixture was stirred at 0° C. for 0.5 h. To the mixture was added 2-(4,6-dibromo-1-oxophthalazin-2(1H)-yl)-N-(5-methylpyrimidin-2-yl)acetamide (200 mg, 0.44 mmol) and the mixture was stirred at 20° C. for 2 h. The mixture was poured into sat. aq. NH₄Cl (10 ml) and extracted with EtOAc (3×10 mL). The combined organic layers were dried over anhydrous Na₂SO₄, filtered, and concentrated under reduced pressure. The residue was purified by reverse-phase preparative HPLC and further purified by chiral SFC (Column: Chiralpak AD-3 (50 mm×4.6 mm, 3 μm particle size); Mobile phase: A: CO₂ B: 0.1% i-PrNH₂ in EtOH; Gradient: B %=54% isocratic; Detection wavelength: 220 nm; Flow rate: 3.4 mL/min; Column temperature: 35° C.; System back pressure: 125 bar) to provide:

2-[6-bromo-4-[trans-2-methylcyclopropyl]oxy-1-oxophthalazin-2-yl]-N-(5-methylpyrimidin-2-yl)acetamide (first eluting isomer, 9): LCMS: m/z=444.0, 446.0 [M+H]⁺. ¹H NMR (400 MHz, CDCl₃): δ 8.92 (br s, 1H), 8.44 (s, 2H), 8.28 (d, J=8.8 Hz, 1H), 8.05 (d, J=1.6 Hz, 1H), 7.88 (dd, J=8.4, 2.0 Hz, 1H), 5.34 (br s, 2H), 3.93-3.86 (m, 1H), 2.28 (s, 3H), 1.23-1.15 (m, 1H), 1.14-1.07 (m, 3H), 1.04-0.95 (m, 1H), 0.68-0.57 (m, 1H).

2-[6-bromo-4-[trans-2-methylcyclopropyl]oxy-1-oxophthalazin-2-yl]-N-(5-methylpyrimidin-2-yl)acetamide (second eluting isomer, 10): LCMS: m/z=444.0, 446.0 [M+H]⁺. ¹H NMR (400 MHz, CDCl₃): δ 8.96 (br s, 1H), 8.45 (s, 2H), 8.28 (d, J=8.8 Hz, 1H), 8.05 (d, J=1.6 Hz, 1H), 7.88 (dd, J=8.4, 2.0 Hz, 1H), 5.34 (br s, 2H), 3.92-3.85 (m, 1H), 2.28 (s, 3H), 1.21-1.13 (m, 1H), 1.13-1.08 (m, 3H), 1.03-0.96 (m, 1H), 0.65-0.58 (m, 1H).

### Example 11

### 2-[6-(1-fluorocyclopropyl)-4-methoxy-1-oxophthalazin-2-yl]-N-(5-fluoropyrimidin-2-yl)acetamide

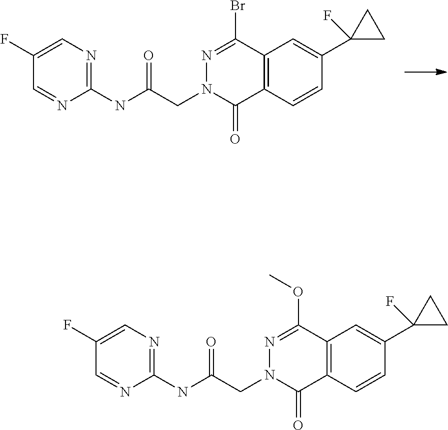

To a solution of NaH (4.0 mg, 0.09 mmol, 60% purity) and in DMF (1.0 mL) at 0° C. was added MeOH (3.0 mg, 0.09 mmol). The mixture was stirred at 0° C. for 0.5 h. To the mixture was added 2-(4-bromo-6-(1-fluorocyclopropyl)-1-oxophthalazin-2(1H)-yl)-N-(5-fluoropyrimidin-2-yl)acetamide (20 mg, 0.04 mmol). The mixture was stirred at 20° C. for 2 h. The mixture was poured into H₂O (5 mL) and extracted with EtOAc (3×2 mL). The combined organic layers were washed with brine (5 mL), dried over anhydrous Na₂SO₄, filtered, and concentrated under reduced pressure. The residue was purified by reverse-phase preparative HPLC. LCMS: m/z=388.0 [M+H]⁺. ¹H NMR (400 MHz, CDCl₃): δ 9.15 (br s, 1H), 8.49 (s, 2H), 8.42 (d, J=8.0 Hz, 1H), 7.84 (s, 1H), 7.63-7.56 (m, 1H), 5.23 (br s, 2H), 4.01 (s, 3H), 1.71-1.65 (m, 2H), 1.26-1.24 (m, 2H).

### Example 12

### 2-[4-cyclopropyloxy-6-(1-fluorocyclopropyl)-1-oxophthalazin-2-yl]-N-(5-fluoropyrimidin-2-yl)acetamide

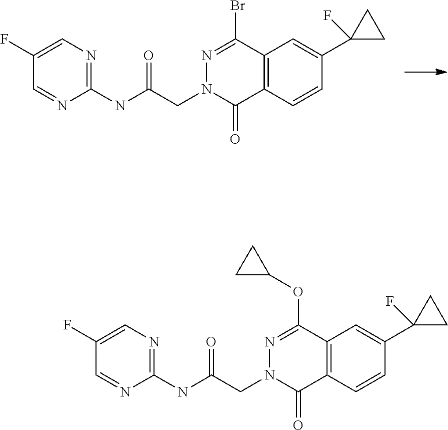

To a solution of NaH (3.0 mg, 0.09 mmol, 60% purity) in DMF (1.0 mL) at 0° C. was added cyclopropanol (5.0 mg, 0.09 mmol). The mixture was stirred at 0° C. for 0.5 h. To the mixture was added 2-(4-bromo-6-(1-fluorocyclopropyl)-1-oxophthalazin-2(1H)-yl)-N-(5-fluoropyrimidin-2-yl)acetamide (20 mg, 0.04 mmol). The mixture was stirred at 20° C. for 2 h. The mixture was poured into H₂O (5 mL) and extracted with EtOAc (3×2 mL). The combined organic layers were washed with brine (5 mL), dried over anhydrous Na₂SO₄, filtered, and concentrated under reduced pressure. The residue was purified by reverse-phase preparative HPLC. LCMS: m/z=414.0 [M+H]⁺. ¹H NMR (400 MHz, CDCl₃): δ 9.12 (br s, 1H), 8.49 (s, 2H), 8.42 (d, J=8.0 Hz, 1H), 7.76 (s, 1H), 7.56 (dd, J=1.6, 8.0 Hz, 1H), 5.24 (br s, 2H), 4.30-4.25 (m, 1H), 1.71-1.63 (m, 2H), 1.27-1.21 (m, 2H), 0.89-0.82 (m, 4H).

### Example 13 and 14

### 2-[4-(cis-2,2-difluorospiro[2.3]hexan-5-yl)oxy-6-(1-fluorocyclopropyl)-1-oxophthalazin-2-yl]-N-(5-fluoropyrimidin-2-yl)acetamide and 2-[4-(trans-2,2-difluorospiro[2.3]hexan-5-yl)oxy-6-(1-fluorocyclopropyl)-1-oxophthalazin-2-yl]-N-(5-fluoropyrimidin-2-yl)acetamide

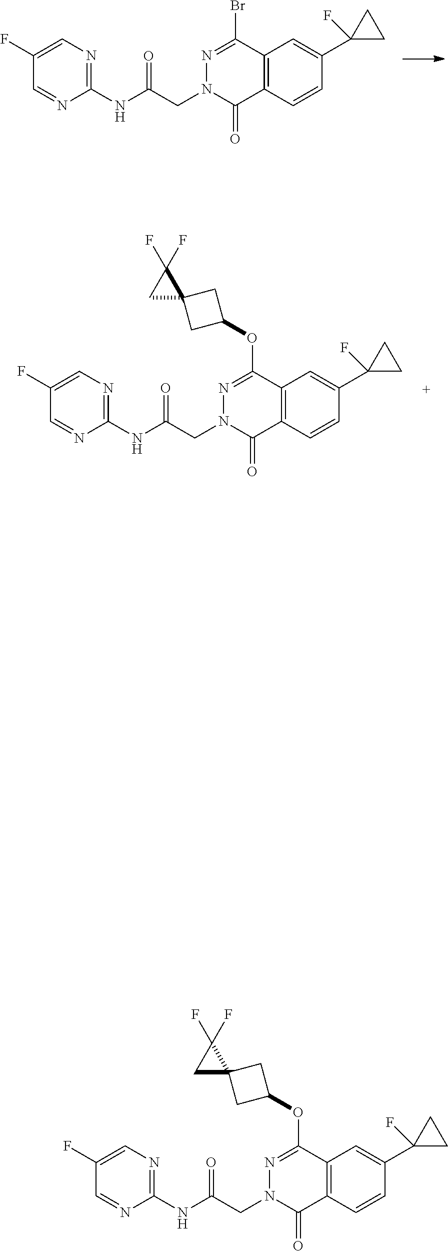

To a solution of NaH (18 mg, 0.46 mmol, 60% purity) in DMF (2 mL) at 0° C. was added 1,1-difluorospiro[2.3]hexan-5-ol (61 mg, 0.46 mmol). The mixture was stirred at 0° C. for 0.5 h. To the mixture was added 2-(4-bromo-6-(1-fluorocyclopropyl)-1-oxophthalazin-2(1H)-yl)-N-(5-fluoropyrimidin-2-yl)acetamide (100 mg, 0.23 mmol) and the mixture was stirred at 20° C. for 1 h. The mixture was poured into H₂O (10 mL) and extracted with EtOAc (3×3 mL). The combined organic layers were washed with brine (10 mL), dried over anhydrous Na₂SO₄, filtered, and concentrated under reduced pressure. The residue was purified by reverse-phase preparative HPLC and further purified by SFC (Column: Chiralpak AD-3 (50 mm×4.6 mm, 3 μm particle size); Mobile phase: A: CO₂ B: 0.1% i-PrNH₂ in i-PrOH; Gradient: B %=50% isocratic; Detection wavelength: 220 nm; Flow rate: 3.4 mL/min; Column temperature: 35° C.; System back pressure: 124 bar) to provide:

2-[4-(2,2-difluorospiro[2.3]hexan-5-yl)oxy-6-(1-fluorocyclopropyl)-1-oxophthalazin-2-yl]-N-(5-fluoropyrimidin-2-yl)acetamide (first eluting isomer, 13): LCMS: m/z=490.1 [M+H]⁺. ¹H NMR (400 MHz, DMSO-d₆) δ 11.08 (br s, 1H), 8.78 (s, 2H), 8.24 (br d, J=8.4 Hz, 1H), 7.88 (s, 1H), 7.70 (br d, J=7.6 Hz, 1H), 5.30-5.27 (m, 1H), 4.99 (br s, 2H), 2.59-2.55 (m, 4H), 1.75-1.60 (m, 2H), 1.53-1.48 (m, 2H), 1.42-1.38 (m, 2H).

2-[4-(2,2-difluorospiro[2.3]hexan-5-yl)oxy-6-(1-fluorocyclopropyl)-1-oxophthalazin-2-yl]-N-(5-fluoropyrimidin-2-yl)acetamide (second eluting isomer, 14): LCMS: m/z=490.1 [M+H]⁺. ¹H NMR (400 MHz, DMSO-d₆) δ 11.17-10.98 (m, 1H), 8.87-8.69 (m, 2H), 8.33-8.17 (m, 1H), 7.96-7.83 (m, 1H), 7.79-7.62 (m, 1H), 5.24-5.10 (m, 1H), 5.07-4.97 (m, 2H), 2.79-2.62 (m, 4H), 1.78-1.61 (m, 2H), 1.59-1.28 (m, 4H).

### Example 15

### 2-[4-(2,2-difluorospiro[3.3]heptan-6-yl)oxy-6-(1-fluorocyclopropyl)-1-oxophthalazin-2-yl]-N-(5-fluoropyrimidin-2-yl)acetamide

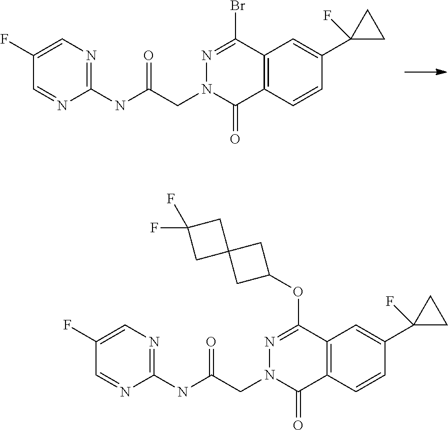

To a mixture of NaH (15 mg, 0.37 mmol, 60% purity) in THF (1.0 mL) was added 2,2-difluorospiro[3.3]heptan-6-ol (82 mg, 0.55 mmol). The mixture was stirred at 0° C. for 0.5 h. To the mixture was added 2-(4-bromo-6-(1-fluorocyclopropyl)-1-oxophthalazin-2(1H)-yl)-N-(5-fluoropyrimidin-2-yl)acetamide (80 mg, 0.18 mmol) at 0° C. and the mixture was then stirred at 20° C. for 2 h. The mixture was diluted with H₂O (10 mL) and extracted with EtOAc (3×3 mL). The combined organic layers were washed with brine (5 mL), dried over anhydrous Na₂SO₄, filtered, and concentrated under reduced pressure. The residue was purified by reverse-phase preparative HPLC. LCMS: m/z=504.1 [M+H]⁺. ¹H NMR (400 MHz, CDCl₃): δ 8.78 (s, 2H), 8.22 (d, J=8.4 Hz, 1H), 7.84 (d, J=1.6 Hz, 1H), 7.67 (dd, J=8.4, 1.6 Hz, 1H), 5.09-4.93 (m, 3H), 2.74-2.58 (m, 6H), 2.41-2.31 (m, 2H), 1.72-1.60 (m, 2H), 1.43-1.34 (m, 2H).

### Example 16

### 2-[6-bromo-4-(2,2-difluorospiro[3.3]heptan-6-yl)oxy-1-oxophthalazin-2-yl]-N-(cis-3-hydroxy-3-methylcyclobutyl)acetamide

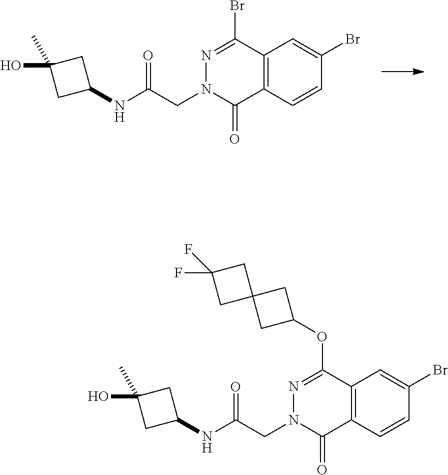 [SMILES: C[C@]1(O)C[C@@H](NC(=O)Cn2nc(Br)c3cc(Br)ccc3c2=O)C1 ; C[C@]1(O)C[C@@H](NC(=O)Cn2nc(OC3CC4(C3)CC(F)(F)C4)c3cc(Br)ccc3c2=O)C1]

To a solution of NaH (31 mg, 0.77 mmol, 60% purity) in DMF (4 mL) at 0° C. was added 6,6-difluorospiro[3.3]heptan-2-ol (115 mg, 0.77 mmol). The mixture was stirred at 0° C. for 0.5 h. 2-(4,6-dibromo-1-oxophthalazin-2(1H)-yl)-N-(cis-3-hydroxy-3-methylcyclobutyl)acetamide (200 mg, 0.38 mmol) was added and the mixture was stirred at 20° C. for 1 h. The mixture was poured into H₂O (10 mL) and extracted with EtOAc (3×3 mL). The combined organic layers were washed with brine (10 mL), dried over anhydrous Na₂SO₄, filtered, and concentrated under reduced pressure. The residue was purified by reverse-phase preparative HPLC. LCMS: m/z=512.0, 514.0 [M+H]⁺. ¹H NMR (400 MHz, CDCl₃): 8.25 (d, J=8.8 Hz, 1H), 8.13 (d, J=1.6 Hz, 1H), 7.92 (dd, J=1.6, 8.4 Hz, 1H), 6.45 (br d, J=7.2 Hz, 1H), 5.12-5.05 (m, 1H), 4.70 (s, 2H), 4.03-3.97 (m, 1H), 2.76-2.60 (m, 6H), 2.54-2.49 (m, 2H), 2.42-2.33 (m, 2H), 2.07-1.96 (m, 2H), 1.37 (s, 3H).

### Example 17 and 18

### 2-[6-bromo-4-[2,2-difluorocyclopropyl]oxy-1-oxophthalazin-2-yl]-N-(5-fluoropyrimidin-2-yl)acetamide

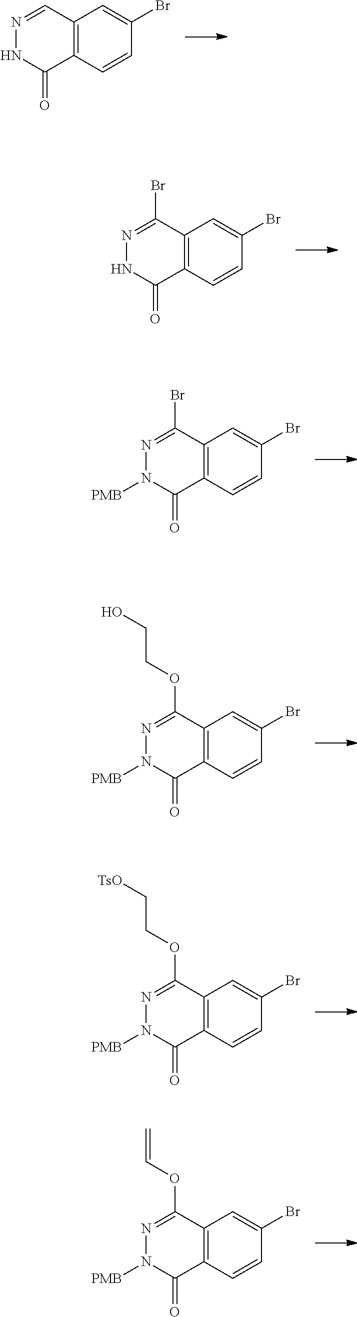

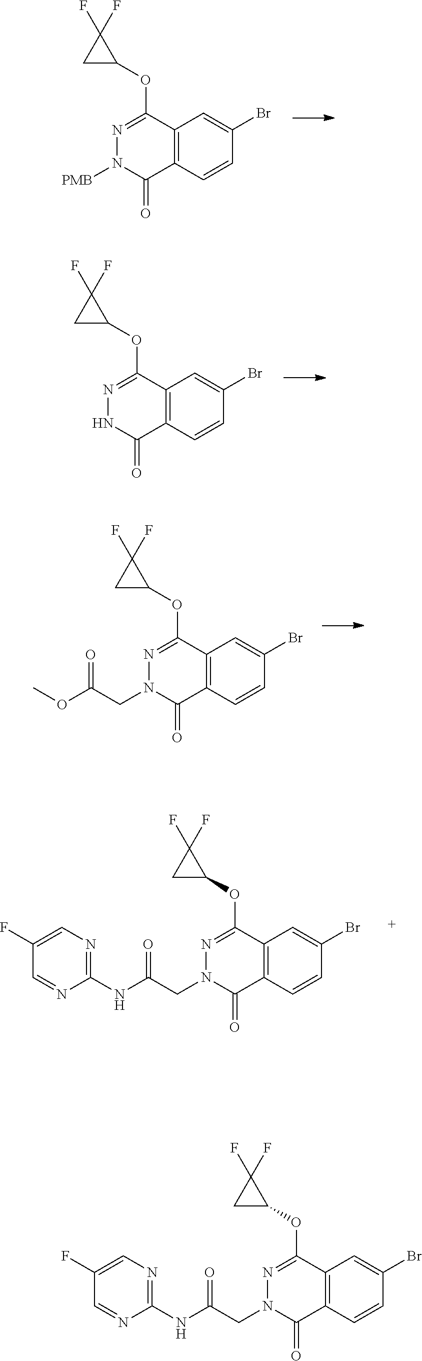

4,6-dibromophthalazin-1(2H)-one: To a mixture of 6-bromophthalazin-1(2H)-one (50 g, 222 mmol) in DMF (500 mL) were added K₂CO₃ (61.4 g, 444 mmol) and benzyltrimethylammonium tribromide (173 g, 444 mmol). The mixture was stirred at 40° C. for 3 h. The mixture was poured into ice-cold H₂O (200 mL) and filtered. The filter cake was dried under reduced pressure to provide a residue that was used directly. LCMS: m/z=302.9, 304.9, 306.9 [M+H]⁺.

4,6-dibromo-2-(4-methoxybenzyl)phthalazin-1(2H)-one: To a mixture of 4,6-dibromophthalazin-1(2H)-one (64 g, 211 mmol) in DMF (100 mL) were added Cs₂CO₃ (137 g, 421 mmol) and PMBCl (37.9 g, 242 mmol). The mixture was stirred for 12 h. The mixture was poured into ice-cold H₂O (200 mL), filtered, and the filter cake was dried under reduced pressure to provide a residue that was used directly. LCMS: m/z=423.0, 424.8, 426.9 [M+H]⁺.

6-bromo-4-(2-hydroxyethoxy)-2-(4-methoxybenzyl)phthalazin-1(2H)-one: To a mixture of ethylene glycol (40.9 g, 660 mmol) in DMF (600 mL) at 0° C. was added NaH (10.6 g, 264 mmol, 60% purity). The mixture was stirred for 30 min at 0° C. The mixture was added a solution of 4,6-dibromo-2-(4-methoxybenzyl)phthalazin-1(2H)-one (56 g, 132 mmol) in DMF (600 mL). The reaction mixture was warmed 25° C. and stirred for 12 h. The mixture was poured into ice-cold H₂O, filtered, and the filter cake was dried under reduced pressure to provide a residue that was used directly. LCMS: m/z=405.0, 407.0 [M+H]⁺.

2-((7-bromo-3-(4-methoxybenzyl)-4-oxo-3,4-dihydrophthalazin-1-yl)oxy)ethyl 4-methylbenzenesulfonate: To a mixture of 6-bromo-4-(2-hydroxyethoxy)-2-(4-methoxybenzyl)phthalazin-1(2H)-one (42 g, 104 mmol) and Et₃N (20.9 g, 207 mmol) in DCM (500 mL) was added 4-methylbenzenesulfonyl chloride (21.7 g, 114 mmol). The mixture was stirred for 12 h. The mixture was poured into ice-cold H₂O (30 mL) and extracted with EtOAc (3×15 mL). The combined organic layers were washed with brine (20 mL), dried over anhydrous Na₂SO₄, filtered, and concentrated under reduced pressure to provide a residue that was used directly. LCMS: m/z=559.0, 561.0 [M+H]⁺.

6-bromo-2-(4-methoxybenzyl)-4-(vinyloxy)phthalazin-1(2H)-one: To a mixture of 2-((7-bromo-3-(4-methoxybenzyl)-4-oxo-3,4-dihydrophthalazin-1-yl)oxy)ethyl 4-methylbenzenesulfonate (40.9 g, 73.1 mmol) in DMSO (400 mL) was added t-BuOK (10.7 g, 95.1 mmol). The mixture was stirred for 1 h. The mixture was poured into aq. sat. NH₄Cl (800 mL), filtered, and the filter cake was dried under reduced pressure. The residue was purified by silica gel column chromatography. LCMS: m/z=387.0, 389.0 [M+H]⁺.

6-bromo-4-(2,2-difluorocyclopropoxy)-2-(4-methoxybenzyl)phthalazin-1(2H)-one: To a solution of 6-bromo-2-(4-methoxybenzyl)-4-(vinyloxy)phthalazin-1(2H)-one (500 mg, 1.29 mmol) and TMSCF₃ (2.75 g, 19.4 mmol) in THF (3.0 mL) was added NaI (580 mg, 3.87 mmol). The mixture was stirred at 110° C. for 20 min. The mixture was poured into H₂O (4 mL) and extracted with EtOAc (3×5 mL). The combined organic layers were dried over anhydrous Na₂SO₄, filtered, and concentrated under reduced pressure to provide a residue that was used directly. LCMS: m/z=437.0, 439.0 [M+H]⁺.

6-bromo-4-(2,2-difluorocyclopropoxy)phthalazin-1(2H)-one: To a solution of 6-bromo-4-(2,2-difluorocyclopropoxy)-2-(4-methoxybenzyl)phthalazin-1(2H)-one (600 mg, 1.37 mmol) in CH₃CN (20 mL) and H₂O (5 mL) was added CAN (3.01 g, 5.49 mmol). The mixture was stirred for 4 h. The mixture was poured into H₂O (5 mL) and extracted with EtOAc (3×5 mL). The combined organic layers were dried over anhydrous Na₂SO₄, filtered, and concentrated under reduced pressure. The residue was purified by silica gel column chromatography. LCMS: m/z=317.0, 319.0 [M+H]⁺.

### methyl 2-(6-bromo-4-(2,2-difluorocyclopropoxy)-1-oxophthalazin-2(1H)-yl)acetate

To a solution of 6-bromo-4-(2,2-difluorocyclopropoxy)phthalazin-1(2H)-one (50 mg, 0.16 mmol) in DMF (3.0 mL) were added Cs₂CO₃ (77 mg, 0.24 mmol) and methyl 2-bromoacetate (29 mg, 0.19 mmol). The mixture was stirred for 16 h. The mixture was poured into brine (5 mL) and extracted with EtOAc (3×5 mL). The combined organic layers were dried over anhydrous Na₂SO₄, filtered, and concentrated under reduced pressure. The residue was purified by silica gel column chromatography. LCMS: m/z=389.0, 391.0 [M+H]⁺.

2-[6-bromo-4-[2,2-difluorocyclopropyl]oxy-1-oxophthalazin-2-yl]-N-(5-fluoropyrimidin-2-yl)acetamide: To a solution of methyl 2-(6-bromo-4-(2,2-difluorocyclopropoxy)-1-oxophthalazin-2(1H)-yl)acetate (60 mg, 0.15 mmol) and 5-fluoropyrimidin-2-amine (21 mg, 0.19 mmol) in DCE (1.0 mL) was added AlMe₃ (1 M in n-hexane, 0.23 mL). The mixture was stirred at 80° C. for 8 h. The mixture was poured into H₂O (5 mL) and extracted with EtOAc (3×5 mL). The organic layers were dried over anhydrous Na₂SO₄, filtered, and concentrated under reduced pressure. The residue was purified by reverse-phase preparative HPLC and further purified by chiral SFC (Column: Daicel Chiralpak AD (250 mm×30 mm, 10 μm particle size); Mobile phase: A: CO₂ B: EtOH; Gradient: B %=54% isocratic; Detection wavelength: 220 nm; Flow rate: 75 g/min; Column temperature: 40° C.; System back pressure: 100 bar) to provide:

2-[6-bromo-4-[2,2-difluorocyclopropyl]oxy-1-oxophthalazin-2-yl]-N-(5-fluoropyrimidin-2-yl)acetamide (first eluting isomer, 17): LCMS: m/z=470.0, 471.9 [M+H]⁺. ¹H NMR (400 MHz, CDCl₃): δ 8.93 (br s, 1H), 8.50 (s, 2H), 8.30 (d, J=8.4 Hz, 1H), 8.10 (d, J=2.0 Hz, 1H), 7.93 (dd, J=2.0, 8.8 Hz, 1H), 5.44 (br d, J=16.4 Hz, 1H), 5.21 (br d, J=15.6 Hz, 1H), 4.57-4.52 (m, 1H), 1.94-1.87 (m, 1H), 1.78-1.73 (m, 1H).

2-[6-bromo-4-[2,2-difluorocyclopropyl]oxy-1-oxophthalazin-2-yl]-N-(5-fluoropyrimidin-2-yl)acetamide (second eluting isomer, 18): LCMS: m/z=469.9, 472.0 [M+H]⁺. ¹H NMR (400 MHz, CDCl₃): δ 8.95 (br s, 1H), 8.50 (s, 2H), 8.30 (d, J=8.4 Hz, 1H), 8.10 (d, J=2.0 Hz, 1H), 7.93 (dd, J=2.0, 8.8 Hz, 1H), 5.45 (br d, J=16.4 Hz, 1H), 5.30-5.13 (m, 1H), 4.60-4.48 (m, 1H), 1.99-1.83 (m, 1H), 1.78-1.73 (m, 1H).

### Example 19 and 20

### 2-[6-bromo-4-(cis-2,2-difluorospiro[2.3]hexan-5-yl)oxy-1-oxophthalazin-2-yl]-N-(3-hydroxy-3-methylcyclobutyl)acetamide and 2-[6-bromo-4-(trans-2,2-difluorospiro[2.3]hexan-5-yl)oxy-1-oxophthalazin-2-yl]-N-(3-hydroxy-3-methylcyclobutyl)acetamide

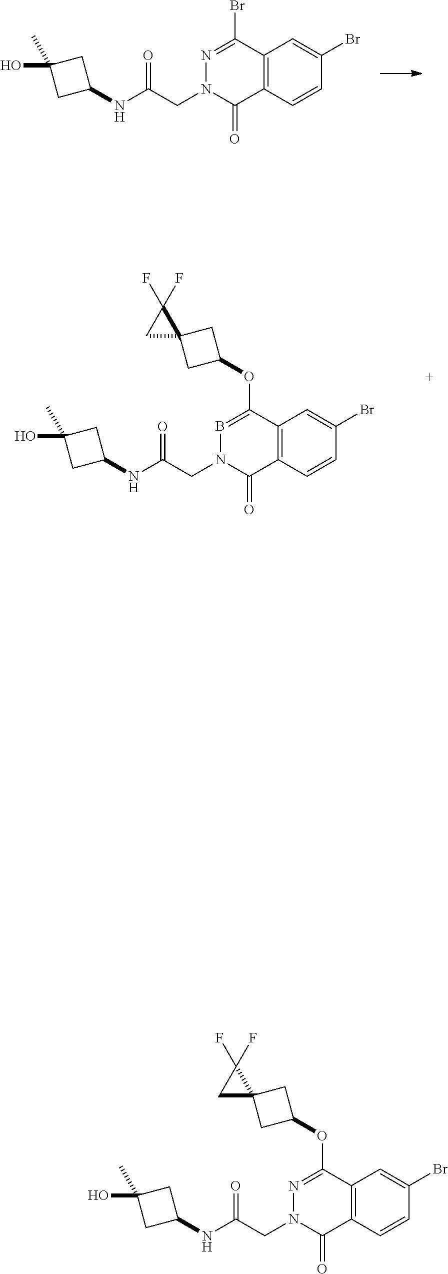

To a solution of NaH (18 mg, 0.45 mmol, 60% purity) in DMF (4.0 mL) at 0° C. was added 1,1-difluorospiro[2.3]hexan-5-ol (60 mg, 0.45 mmol). The mixture was stirred at 0° C. for 0.5 h. To the mixture was added 2-(4,6-dibromo-1-oxophthalazin-2(1H)-yl)-N-(cis-3-hydroxy-3-methylcyclobutyl)acetamide (100 mg, 0.22 mmol) at 0° C. The mixture was stirred at 20° C. for 1 h. The mixture was poured into H₂O (5 mL) and extracted with EtOAc (3×3 mL). The combined organic layers were washed with brine, dried over anhydrous Na₂SO₄, filtered, and concentrated under reduced pressure.

The residue was purified by reverse-phase preparative HPLC and further purified by SFC (Column: Chiralpak AD-3 (50 mm×4.6 mm, 3 μm particle size); Mobile phase: A: CO₂ B: 0.1% i-PrNH₂ in EtOH; Gradient: B %=25% isocratic; Detection wavelength: 220 nm; Flow rate: 3.4 mL/min; Column temperature: 35° C.; System back pressure: 124 bar) to provide: [0183]2-[6-bromo-4-(2,2-difluorospiro[2.3]hexan-5-yl)oxy-1-oxophthalazin-2-yl]-N-(cis-3-hydroxy-3-methylcyclobutyl)acetamide (first eluting isomer, 19): LCMS: m/z=498.0, 500.0 [M+H]⁺. ¹H NMR (400 MHz, CDCl₃) δ 8.22 (d, J=8.4 Hz, 1H), 8.15 (s, 1H), 7.92 (dd, J=1.6, 8.4 Hz, 1H), 6.56 (br d, J=7.2 Hz, 1H), 5.39-5.32 (m, 1H), 4.69 (s, 2H), 4.03-3.94 (m, 1H), 2.69-2.62 (m, 2H), 2.61-2.54 (m, 2H), 2.52-2.45 (m, 2H), 2.03-1.98 (m, 2H), 1.41-1.31 (m, 5H). [0184]2-[6-bromo-4-(2,2-difluorospiro[2.3]hexan-5-yl)oxy-1-oxophthalazin-2-yl]-N-(cis-3-hydroxy-3-methylcyclobutyl)acetamide (second eluting isomer, 20): LCMS: m/z=498.0, 500.0 [M+H]⁺. ¹H NMR (400 MHz, CDCl₃) δ 8.25 (d, J=8.4 Hz, 1H), 8.16 (d, J=1.6 Hz, 1H), 7.92 (dd, J=1.6, 8.4 Hz, 1H), 6.41 (br d, J=6.8 Hz, 1H), 5.22 (t, J=6.8 Hz, 1H), 4.70 (s, 2H), 4.08-3.93 (m, 1H), 2.88-2.73 (m, 2H), 2.58-2.40 (m, 4H), 2.05-1.98 (m, 2H), 1.37 (s, 3H), 1.31 (t, J=8.2 Hz, 2H).

### Example 21 and 22

### 2-[6-bromo-4-(cis-2,2-difluorospiro[2.3]hexan-5-yl)oxy-1-oxophthalazin-2-yl]-N-(5-methylpyrimidin-2-yl)acetamide and 2-[6-bromo-4-(trans-2,2-difluorospiro[2.3]hexan-5-yl)oxy-1-oxophthalazin-2-yl]-N-(5-methylpyrimidin-2-yl)acetamide

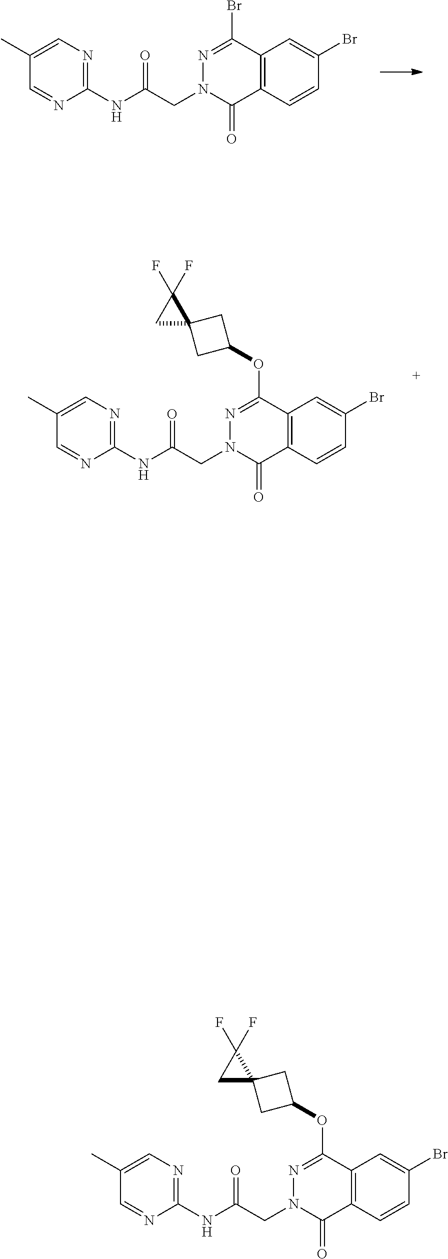

To a solution of NaH (18 mg, 0.44 mmol, 60% purity) in DMF (1.0 mL) at 0° C. was added 1,1-difluorospiro[2.3]hexan-5-ol (60 mg, 0.44 mmol). The mixture was stirred at 0° C. for 0.5 h. To the mixture was added 2-(4,6-dibromo-1-oxophthalazin-2(1H)-yl)-N-(5-methylpyrimidin-2-yl)acetamide (100 mg, 0.22 mmol) and the mixture was stirred at 20° C. for 1 h. The mixture was poured into H₂O (10 mL) and extracted with EtOAc (3×3 mL). The combined organic layers were washed with brine (10 mL), dried over anhydrous Na₂SO₄, filtered, and concentrated under reduced pressure. The residue was purified by reverse-phase preparative HPLC and further purified by SFC (Column: Chiralpak AD-3 (50 mm×4.6 mm, 3 μm particle size); Mobile phase: A: CO₂ B: 0.1% i-PrNH₂ in EtOH; Gradient: B %=40% isocratic; Detection wavelength: 220 nm; Flow rate: 4 mL/min; Column temperature: 35° C.; System back pressure: 124 bar) to provide:

2-[6-bromo-4-(2,2-difluorospiro[2.3]hexan-5-yl)oxy-1-oxophthalazin-2-yl]-N-(5-methylpyrimidin-2-yl)acetamide (first eluting isomer, 21): LCMS: m/z=506.0, 508.0 [M+H]⁺. ¹H NMR (400 MHz, DMSO-d₆): δ 10.82 (s, 1H), 8.52 (s, 2H), 8.24-8.07 (m, 3H), 5.30-5.24 (m, 1H), 5.01 (br s, 2H), 2.57 (br d, J=5.6 Hz, 4H), 2.22 (s, 3H), 1.50 (br t, J=8.4 Hz, 2H).

2-[6-bromo-4-(2,2-difluorospiro[2.3]hexan-5-yl)oxy-1-oxophthalazin-2-yl]-N-(5-methylpyrimidin-2-yl)acetamide (second eluting isomer, 22): LCMS: m/z=506.0, 508.0 [M+H]⁺. ¹H NMR (400 MHz, DMSO-d₆) δ 10.82 (s, 1H), 8.50 (s, 2H), 8.19-8.07 (m, 3H), 5.13-5.10 (m, 1H), 4.99 (s, 2H), 2.70-2.61 (m, 2H), 2.46 (br d, J=4.0 Hz, 2H), 2.21 (s, 3H), 1.44 (t, J=8.6 Hz, 2H).

### Example 23

### 2-(6-bromo-4-cyclopropyloxy-1-oxophthalazin-2-yl)-N-(cis-3-hydroxy-3-methylcyclobutyl)acetamide

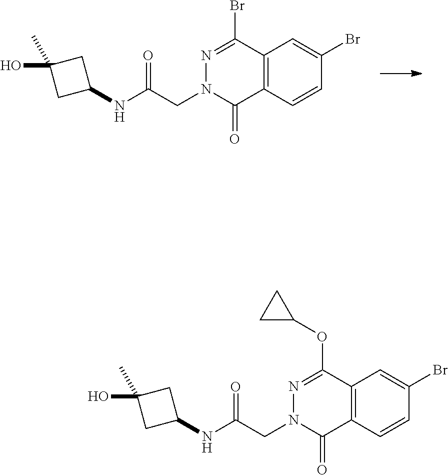 [SMILES: C[C@]1(O)C[C@@H](NC(=O)Cn2nc(Br)c3cc(Br)ccc3c2=O)C1 ; C[C@]1(O)C[C@@H](NC(=O)Cn2nc(OC3CC3)c3cc(Br)ccc3c2=O)C1]

To a solution of NaH (18 mg, 0.45 mmol, 60% purity) in DMF (2.0 mL) at 0° C. was added cyclopropanol (26 mg, 0.45 mmol). The mixture was stirred at 0° C. for 0.5 h. To the mixture was added 2-(4,6-dibromo-1-oxophthalazin-2(1H)-yl)-N-(cis-3-hydroxy-3-methylcyclobutyl)acetamide (100 mg, 0.22 mmol) at 0° C. and the mixture was stirred at 20° C. for 1.5 h. The mixture was poured into H₂O (5 mL) and extracted with EtOAc (3×2 mL). The combined organic layers were washed with brine (3 mL), dried over anhydrous Na₂SO₄, filtered, and concentrated under reduced pressure. The residue was purified by reverse-phase preparative HPLC. LCMS: m/z=422.0, 424.0 [M+H]⁺. ¹H NMR (400 MHz, CDCl₃): δ 8.22 (d, J=8.4 Hz, 1H), 8.04 (d, J=1.6 Hz, 1H), 7.89 (dd, J=8.4, 1.6 Hz, 1H), 6.60 (br d, J=7.2 Hz, 1H), 4.76 (s, 2H), 4.33-4.21 (m, 1H), 4.10-3.90 (m, 1H), 2.40-2.59 (m, 2H), 2.28 (s, 1H), 2.11-1.93 (m, 2H), 1.36 (s, 3H), 0.85 (d, J=4.8 Hz, 4H).

### Example 24

### 2-(6-bromo-4-methoxy-1-oxophthalazin-2-yl)-N-(cis-3-hydroxy-3-methylcyclobutyl)acetamide

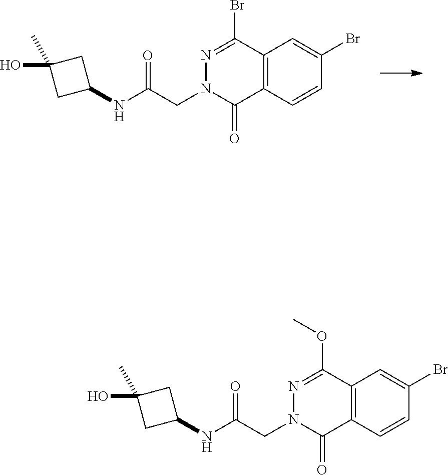 [SMILES: C[C@]1(O)C[C@@H](NC(=O)Cn2nc(Br)c3cc(Br)ccc3c2=O)C1 ; COc1nn(CC(=O)N[C@H]2C[C@@](C)(O)C2)c(=O)c2ccc(Br)cc12]

To a solution of NaH (18 mg, 0.45 mmol, 60% purity) in DMF (2.0 mL) at 0° C. was added MeOH (14 mg, 0.45 mmol). The mixture was stirred at 0° C. for 0.5 h. To the mixture was added 2-(4,6-dibromo-1-oxophthalazin-2(1H)-yl)-N-(cis-3-hydroxy-3-methylcyclobutyl)acetamide (100 mg, 0.22 mmol) at 0° C. and the mixture was stirred at 20° C. for 2 h. The mixture was poured into H₂O (5 mL) and extracted with EtOAc (3×2 mL). The combined organic layers were washed with brine (3 mL), dried over anhydrous Na₂SO₄, filtered, and concentrated under reduced pressure. The residue was purified by reverse-phase preparative HPLC. LCMS: m/z=396.0, 398.0 [M+H]⁺. ¹H NMR (400 MHz, CDCl₃): δ 8.26 (d, J=8.4 Hz, 1H), 8.16 (d, J=2.0 Hz, 1H), 7.92 (dd, J=8.4, 2.0 Hz, 1H), 6.48 (br d, J=6.8 Hz, 1H), 4.74 (s, 2H), 4.08-3.95 (m, 4H), 2.61-2.45 (m, 2H), 2.08-1.97 (m, 3H), 1.37 (s, 3H).

### Example 25 and 26

### 2-[6-bromo-4-(cis-2,2-difluorospiro[2.3]hexan-5-yl)oxy-1-oxophthalazin-2-yl]-N-([1,2,4]triazolo[1,5-a]pyridin-2-yl)acetamide and 2-[6-bromo-4-(trans-2,2-difluorospiro[2.3]hexan-5-yl)oxy-1-oxophthalazin-2-yl]-N-([1,2,4]triazolo[1,5-a]pyridin-2-yl)acetamide

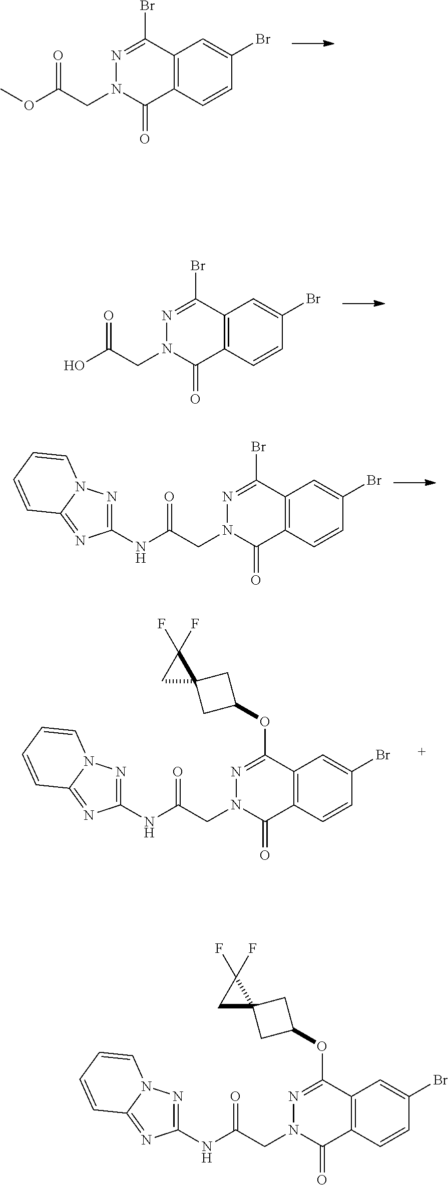

2-(4,6-dibromo-1-oxophthalazin-2(1H)-yl)acetic acid: To a solution of methyl 2-(4,6-dibromo-1-oxophthalazin-2(1H)-yl)acetate (5.0 g, 13.3 mmol) in THF (50 mL) and H₂O (10 mL) was added LiOH·H₂O (1.12 g, 26.6 mmol). The mixture was stirred for 2 h. The reaction mixture was poured into H₂O (50 mL) and the mixture was acidified with aq. HCl (3M) to pH=2. The mixture was extracted with EtOAc (3×50 mL) and the combined organic layers were dried over anhydrous Na₂SO₄, filtered, and concentrated under reduced pressure. The residue was purified by reverse-phase preparative HPLC. LCMS: m/z=360.9, 362.9, 364.9 [M+H]⁺.

N-([1,2,4]triazolo[1,5-a]pyridin-2-yl)-2-(4,6-dibromo-1-oxophthalazin-2(1H)-yl)acetamide: To a solution of 2-(4,6-dibromo-1-oxophthalazin-2(1H)-yl)acetic acid (200 mg, 0.55 mmol) and [1,2,4]triazolo[1,5-a]pyridin-2-amine (222 mg, 1.66 mmol) in pyridine (2 mL) was added a solution of EDCI (318 mg, 1.66 mmol) in pyridine (2 mL). The mixture was stirred for 1.5 h. The mixture was poured into H₂O (15 mL), filtered, and the filter cake was dried under reduced pressure. The residue was triturated with MeCN (5 mL), filtered, and dried under reduced pressure to provide a residue that was used directly. LCMS: m/z=477.0, 479.0, 481.0 [M+H]⁺.

2-[6-bromo-4-(cis-2,2-difluorospiro[2.3]hexan-5-yl)oxy-1-oxophthalazin-2-yl]-N-([1,2,4]triazolo[1,5-a]pyridin-2-yl)acetamide and 2-[6-bromo-4-(trans-2,2-difluorospiro[2.3]hexan-5-yl)oxy-1-oxophthalazin-2-yl]-N-([1,2,4]triazolo[1,5-a]pyridin-2-yl)acetamide: To a solution of NaH (17 mg, 0.42 mmol, 60% purity) in DMF (4 mL) at 0° C. was added 1,1-difluorospiro[2.3]hexan-5-ol (56 mg, 0.42 mmol). The mixture was stirred at 0° C. for 0.5 h. To the mixture was added N-([1,2,4]triazolo[1,5-a]pyridin-2-yl)-2-(4,6-dibromo-1-oxophthalazin-2(1H)-yl)acetamide (100 mg, 0.21 mmol) at 0° C. and the mixture was stirred at 15° C. for 16 h. The mixture was poured into H₂O (10 mL) and extracted with EtOAc (3×5 mL). The combined organic layers were dried over anhydrous Na₂SO₄, filtered, and concentrated under reduced pressure. The residue was purified by reverse-phase preparative HPLC and further purified by SFC (Column: Daicel Chiralpak AD (250 mm×30 mm, 10 μm particle size); Mobile phase: A: CO₂ B: 0.1% NH₃—H₂O in i-PrOH; Gradient: B %=50% isocratic; Detection wavelength: 220 nm; Flow rate: 3.4 mL/min; Column temperature: 35° C.; System back pressure: 124 bar) to provide:

2-[6-bromo-4-(2,2-difluorospiro[2.3]hexan-5-yl)oxy-1-oxophthalazin-2-yl]-N-([1,2,4]triazolo[1,5-a]pyridin-2-yl)acetamide (first eluting isomer, 25): LCMS: m/z=531.0, 533.0 [M+H]¹. ¹H NMR (400 MHz, DMSO-d₆): δ 11.14 (br s, 1H), 8.85 (d, J=6.8 Hz, 1H), 8.20 (d, J=1.6 Hz, 1H), 8.19-8.15 (m, 1H), 8.14-8.10 (m, 1H), 7.73-7.68 (m, 1H), 7.67-7.61 (m, 1H), 7.14 (dt, J=1.2, 6.8 Hz, 1H), 5.32-5.25 (m, 1H), 4.97 (br s, 2H), 2.75-2.53 (m, 4H), 1.50 (t, J=8.4 Hz, 2H).

2-[6-bromo-4-(2,2-difluorospiro[2.3]hexan-5-yl)oxy-1-oxophthalazin-2-yl]-N-([1,2,4]triazolo[1,5-a]pyridin-2-yl)acetamide (second eluting isomer, 26): LCMS: m/z=531.0, 533.0 [M+H]⁺. ¹H NMR (400 MHz, DMSO-d₆): δ 11.13 (s, 1H), 8.84 (d, J=6.8 Hz, 1H), 8.20 (d, J=1.6 Hz, 1H), 8.19-8.15 (m, 1H), 8.14-8.09 (m, 1H), 7.73-7.68 (m, 1H), 7.67-7.61 (m, 1H), 7.14 (dt, J=1.2, 6.8 Hz, 1H), 5.20-5.11 (m, 1H), 4.97 (br s, 2H), 2.73-2.64 (m, 2H), 2.55-2.51 (m, 2H), 1.47 (t, J=8.4 Hz, 2H).

### Example 27

### 2-(6-bromo-4-cyclopropyloxy-1-oxophthalazin-2-yl)-N-([1,2,4]triazolo[1,5-a]pyridin-2-yl)acetamide

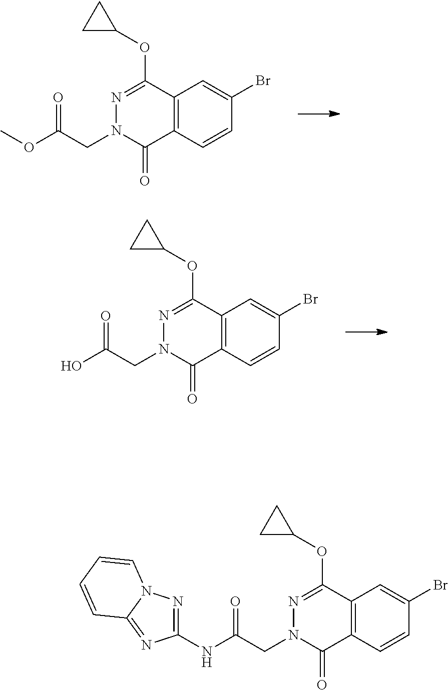

2-[6-bromo-4-(cyclopropoxy)-1-oxo-phthalazin-2-yl]acetic acid: To a solution of methyl 2-[6-bromo-4-(cyclopropoxy)-1-oxo-phthalazin-2-yl]acetate (490 mg, 1.4 mmol) in THF (5.7 mL) was added aq. LiOH (2.7 mL, 1 M). The mixture was stirred at 40° C. for 2 h. The mixture was diluted with aq. HCl (3.5 mL, 1 M) and extracted with EtOAc (2×10 mL). The combined organic layers were washed with brine (10 mL), dried over anhydrous Na₂SO₄, filtered, and concentrated under reduced pressure to provide a residue that was used directly. LCMS: m/z=339.1, 341.1 [M+H]⁺.

2-(6-bromo-4-cyclopropyloxy-1-oxophthalazin-2-yl)-N-([1,2,4]triazolo[1,5-a]pyridin-2-yl)acetamide: To a solution of 2-(6-bromo-4-cyclopropoxy-1-oxophthalazin-2(1H)-yl)acetic acid (100 mg, 0.30 mmol) in pyridine (2.0 mL) were added [1,2,4]triazolo[1,5-a]pyridin-2-amine (99 mg, 0.74 mmol) and EDCI (113 mg, 0.59 mmol). The mixture was stirred for 3 h. The reaction mixture was diluted with H₂O (20 mL) and extracted with EtOAc (3×3 mL). The combined organic layers were washed with brine (3×10 mL) dried over anhydrous Na₂SO₄, filtered, and concentrated under reduced pressure. The residue was purified by reverse-phase preparative HPLC. LCMS: m/z=455.0, 456.9 [M+H]⁺. ¹H NMR (400 MHz, DMSO-d₆): δ 11.16 (br s, 1H), 8.87 (d, J=6.8 Hz, 1H), 8.19-8.06 (m, 3H), 7.73-7.64 (m, 2H), 7.17-7.14 (m, 1H), 5.11-4.90 (m, 2H), 4.27-4.25 (m, 1H), 0.85-0.79 (m, 4H).

### Example 28

### 2-(4-cyclopropyloxy-6-iodo-1-oxophthalazin-2-yl)-N-(5-fluoropyrimidin-2-yl)acetamide

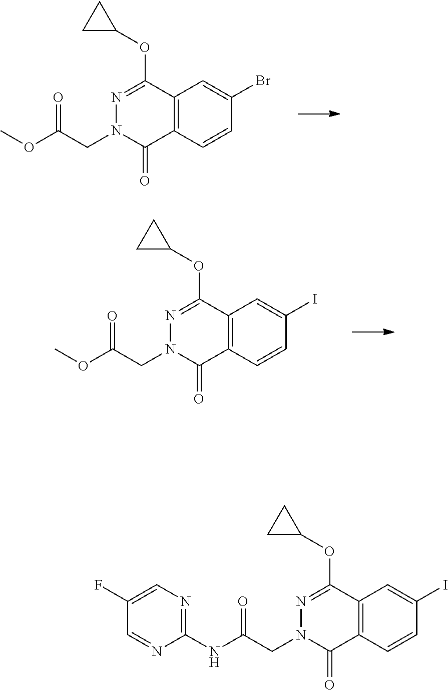

methyl 2-(4-cyclopropoxy-6-iodo-1-oxophthalazin-2(1H)-yl)acetate: To a solution of methyl 2-(6-bromo-4-cyclopropoxy-1-oxophthalazin-2(1H)-yl)acetate (500 mg, 1.42 mmol) in 1,4-dioxane (5 mL) were added NaI (2.12 g, 14.2 mmol), (1R,2R)—N¹,N²-dimethylcyclohexane-1,2-diamine (101 mg, 0.70 mmol), and CuI (270 mg, 1.42 mmol). The mixture was stirred at 110° C. for 12 h. The reaction mixture was quenched by addition of H₂O (5 mL) and extracted with EtOAc (3×5 mL). The combined organic layers were washed with brine (10 mL), dried over anhydrous Na₂SO₄, filtered, and concentrated under reduced pressure. The residue was purified by silica gel column chromatography. LCMS: m/z=401.1 [M+H]⁺.

2-(4-cyclopropyloxy-6-iodo-1-oxophthalazin-2-yl)-N-(5-fluoropyrimidin-2-yl)acetamide: To a mixture of 5-fluoropyrimidin-2-amine (28 mg, 0.25 mmol) and methyl 2-(4-cyclopropoxy-6-iodo-1-oxophthalazin-2(1H)-yl)acetate (50 mg, 0.12 mmol) in DCE (2.0 mL) was added AlMe₃ (1 M in n-heptane, 0.38 mL). The mixture was stirred for 6 h at 60° C. The reaction mixture was cooled to 0° C., diluted with H₂O (15 mL), and extracted with EtOAc (4×10 mL). The combined organic layers were washed with brine (2×10 mL), dried over anhydrous Na₂SO₄, filtered, and concentrated under reduced pressure. The residue was purified by reverse-phase preparative HPLC. LCMS: m/z=481.9 [M+H]⁺. ¹H NMR (400 MHz, DMSO-d₆): δ 11.10 (br s, 1H), 8.78 (s, 2H), 8.27-8.21 (m, 2H), 7.96 (d, J=8.4 Hz, 1H), 5.01 (s, 2H), 4.28-4.19 (m, 1H), 0.86-0.75 (m, 4H).

### Example 29

### 2-(6-bromo-1-oxo-4-spiro[2.3]hexan-5-yloxyphthalazin-2-yl)-N-(5-methylpyrimidin-2-yl)acetamide

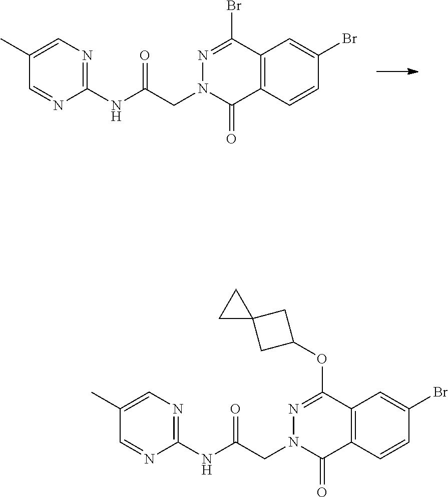 [SMILES: Cc1cnc(NC(=O)Cn2nc(Br)c3cc(Br)ccc3c2=O)nc1 ; Cc1cnc(NC(=O)Cn2nc(OC3CC4(CC4)C3)c3cc(Br)ccc3c2=O)nc1]

To a solution of NaH (53 mg, 1.32 mmol, 60% purity) in DMF (1 mL) at 0° C. was added spiro[2.3]hexan-5-ol (43 mg, 0.44 mmol). The mixture was stirred at 0° C. for 45 min. To the mixture was added 2-(4,6-dibromo-1-oxo-phthalazin-2-yl)-N-(5-methylpyrimidin-2-yl)acetamide (100 mg, 0.22 mmol) and the mixture was stirred for 2 h at 0° C. The reaction mixture was poured into H₂O (15 mL) and extracted with EtOAc (3×15 mL). The combined organic layers were dried over anhydrous Na₂SO₄, filtered, and concentrated under reduced pressure. The residue was purified by reverse-phase preparative HPLC. LCMS: m/z=470.0, 472.0 [M+H]⁺. ¹H NMR (400 MHz, CDCl₃) δ 8.66 (br s, 1H), 8.42 (s, 2H), 8.29 (d, J=8.4 Hz, 1H), 8.18 (d, J=2.0 Hz, 1H), 7.90 (dd, J=2.0 Hz, 1H), 5.38-5.21 (m, 3H), 2.57-2.41 (m, 4H), 2.27 (s, 3H), 0.56-0.44 (m, 4H).

### Example 30

### 2-(6-bromo-1-oxo-4-spiro[2.3]hexan-5-yloxyphthalazin-2-yl)-N-(5-cyanopyrimidin-2-yl)acetamide

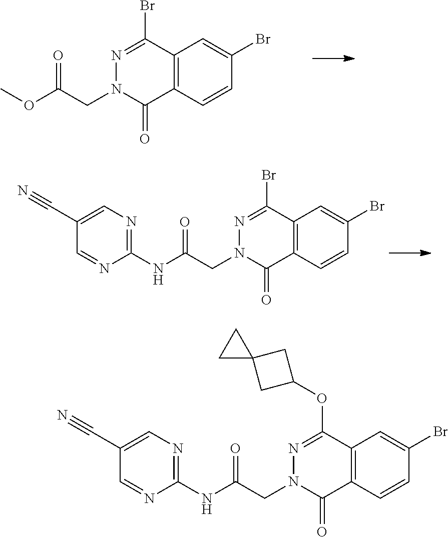

N-(5-cyanopyrimidin-2-yl)-2-(4,6-dibromo-1-oxophthalazin-2(1H)-yl)acetamide: To a solution of methyl 2-(4,6-dibromo-1-oxophthalazin-2(1H)-yl)acetate (300 mg, 0.80 mmol) in DCE (10 mL) were added 2-aminopyrimidine-5-carbonitrile (287 mg, 2.39 mmol) and AlMe₃ (1 M in n-heptane, 2.39 mL). The mixture was stirred at 60° C. for 2 h. The reaction was quenched with H₂O (6 mL) and extracted with EtOAc (3×6 mL). The combined organic layers are washed with brine (6 mL), dried over anhydrous Na₂SO₄, filtered, and concentrated under reduced pressure to provide a residue that was used directly. LCMS: m/z=462.9, 464.9, 466.9 [M+H]⁺.

2-(6-bromo-1-oxo-4-spiro[2.3]hexan-5-yloxyphthalazin-2-yl)-N-(5-cyanopyrimidin-2-yl)acetamide: To a solution of NaH (52 mg, 1.29 mmol, 60% purity) in DMF (2.0 mL) at 0° C. was added spiro[2.3]hexan-5-ol (42 mg, 0.43 mmol). The mixture was stirred at 0° C. for 45 min. To the mixture was added N-(5-cyanopyrimidin-2-yl)-2-(4,6-dibromo-1-oxophthalazin-2(1H)-yl)acetamide (100 mg, 0.22 mmol) at 0° C. and the mixture was stirred at 20° C. for 2 h. The reaction mixture was diluted with aq. sat. NH₄Cl (10 mL) and extracted with EtOAc (4×5 mL). The combined organic layers were washed with brine (10 mL), dried over anhydrous Na₂SO₄, filtered, and concentrated under reduced pressure. The residue was purified by reverse-phase preparative HPLC. LCMS: m/z=481.0, 482.9 [M+H]⁺. ¹H NMR (400 MHz, CDCl₃): δ 11.50 (br s, 1H), 9.14 (s, 2H), 8.16 (d, J=8.0 Hz, 2H), 8.12-8.09 (m, 1H), 5.25-5.20 (m, 1H), 5.07 (s, 2H), 2.47-2.39 (m, 4H), 0.50-0.45 (m, 4H).

### Example 31

### 2-(6-cyclopropyl-5-fluoro-1-oxo-4-spiro[2.3]hexan-5-yloxyphthalazin-2-yl)-N-(5-fluoropyrimidin-2-yl)acetamide

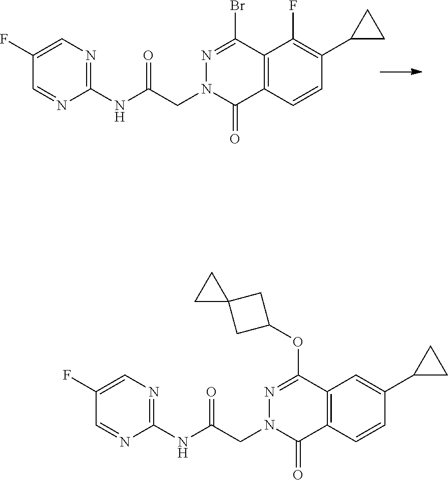 [SMILES: O=C(Cn1nc(Br)c2c(F)c(C3CC3)ccc2c1=O)Nc1ncc(F)cn1 ; O=C(Cn1nc(OC2CC3(CC3)C2)c2cc(C3CC3)ccc2c1=O)Nc1ncc(F)cn1]

To a solution of NaH (9 mg, 0.23 mmol, 60% purity) in DMF (2.0 mL) at 0° C. was added spiro[2.3]hexan-5-ol (22.50 mg, 0.23 mmol). The mixture was stirred at 0° C. for 0.5 h. Then 2-(4-bromo-6-cyclopropyl-5-fluoro-1-oxophthalazin-2(1H)-yl)-N-(5-fluoropyrimidin-2-yl)acetamide (50 mg, 0.12 mmol) was added. The mixture was stirred at 20° C. for 2 h. The reaction mixture was diluted with aq. sat. NH₄Cl (10 mL) and extracted with EtOAc (4×5 mL). The combined organic layers were washed with brine (10 mL), dried over anhydrous Na₂SO₄, filtered, and concentrated under reduced pressure. The residue was purified by reverse-phase preparative HPLC. LCMS: m/z=454.1 [M+H]⁺. ¹H NMR (400 MHz, CDCl₃): δ 9.45 (s, 1H), 8.49 (s, 2H), 8.13 (d, J=8.24 Hz, 1H), 7.30-7.22 (m, 1H), 5.34-5.30 (m, 1H), 5.19 (br s, 2H), 2.59-2.40 (m, 4H), 2.36-2.24 (m, 1H), 1.19-1.11 (m, 2H), 0.89-0.81 (m, 2H), 0.55-0.41 (m, 4H).

### Example 32 and 33

### 2-(6-cyclopropyl-5-fluoro-4-methoxy-1-oxophthalazin-2-yl)-N-(5-fluoropyrimidin-2-yl)acetamide and 2-(6-cyclopropyl-4,5-dimethoxy-1-oxophthalazin-2-yl)-N-(5-fluoropyrimidin-2-yl)acetamide

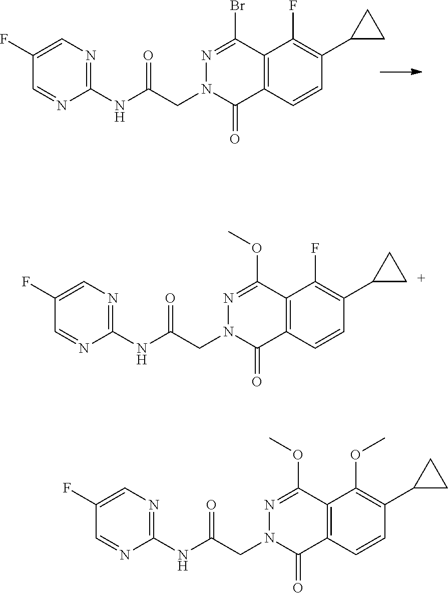

To a solution of NaH (9 mg, 0.23 mmol, 60% purity) in DMF (1.0 mL) at 0° C. was added MeOH (7 mg, 0.23 mmol). The mixture was stirred at 0° C. for 0.5 h. To the mixture was added a solution of 2-(4-bromo-6-cyclopropyl-5-fluoro-1-oxophthalazin-2(1H)-yl)-N-(5-fluoropyrimidin-2-yl)acetamide (50 mg, 0.12 mmol) in DMF (1.0 mL) at 0° C. The mixture was stirred at 20° C. for 2 h. The mixture was diluted with H₂O (10 mL) and extracted with EtOAc (3×2 mL). The combined organic layers were washed with brine (5 mL), dried over anhydrous Na₂SO₄, filtered, and concentrated under reduced pressure. The residue was purified by reverse-phase preparative HPLC to provide:

2-(6-cyclopropyl-5-fluoro-4-methoxy-1-oxophthalazin-2-yl)-N-(5-fluoropyrimidin-2-yl)acetamide (32): LCMS: m/z=388.0 [M+H]⁺. ¹H NMR (400 MHz, CDCl₃): δ 9.32 (br s, 1H), 8.51 (s, 2H), 8.17 (d, J=8.4 Hz, 1H), 7.31-7.29 (m, 1H), 5.25 (br s, 2H), 4.01 (s, 3H), 2.38-2.25 (m, 1H), 1.22-1.13 (m, 2H), 0.92-0.85 (m, 2H).

2-(6-cyclopropyl-4,5-dimethoxy-1-oxophthalazin-2-yl)-N-(5-fluoropyrimidin-2-yl)acetamide (33): LCMS: m/z=400.0 [M+H]⁺. ¹H NMR (400 MHz, CDCl₃): δ 9.24 (br s, 1H), 8.48 (s, 2H), 8.18 (d, J=8.4 Hz, 1H), 7.20 (d, J=8.4 Hz, 1H), 5.18 (br s, 2H), 4.02 (s, 3H), 3.91 (s, 3H), 2.55-2.36 (m, 1H), 1.20-1.11 (m, 2H), 0.87-0.78 (m, 2H).

### Example 34

### 2-(6-chloro-4-cyclopropyloxy-1-oxophthalazin-2-yl)-N-(5-fluoropyrimidin-2-yl)acetamide

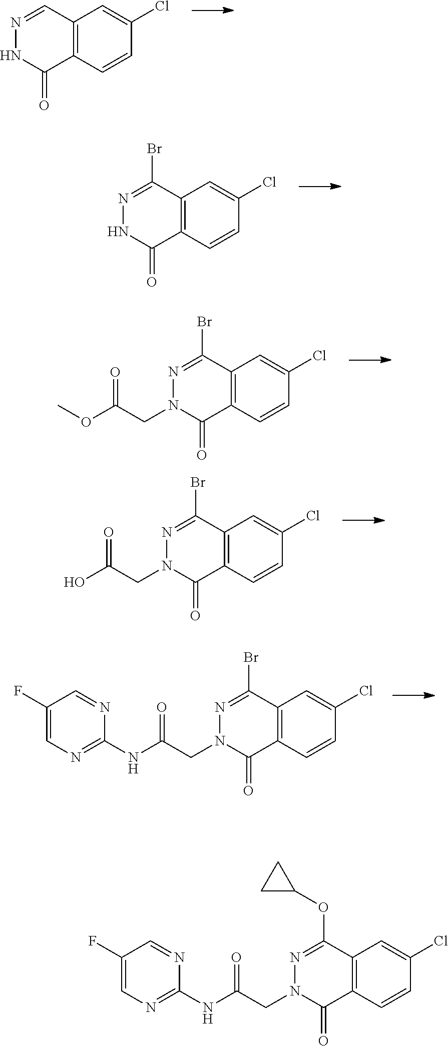

4-bromo-6-chlorophthalazin-1(2H)-one: To a solution of 6-chlorophthalazin-1(2H)-one (3.0 g, 16.6 mmol) in DMF (50 mL) was added K₂CO₃ (4.59 g, 33.2 mmol) and mono(N,N,N-trimethyl-1-phenylmethanaminium) tribromide (12.9 g, 33.2 mmol). The mixture was stirred at 40° C. for 4 h. The mixture was diluted with H₂O (20 mL), filtered, and the filter cake was triturated with H₂O (3×50 mL) and dried under reduced pressure. The residue was triturated with MeCN (150 mL) and dried under reduced pressure to provide a residue that was used directly. LCMS: m/z=259.0, 261.0, 263.0 [M+H]⁺.

methyl 2-(4-bromo-6-chloro-1-oxophthalazin-2(1H)-yl)acetate: To a solution of 4-bromo-6-chlorophthalazin-1(2H)-one (4.9 g, 18.9 mmol) in DMF (70 mL) were added Cs₂CO₃ (12.31 g, 37.8 mmol) and methyl 2-bromoacetate (3.47 g, 22.7 mmol). The mixture was stirred for 4 h. The mixture was diluted with H₂O (4 mL) and extracted with EtOAc (3×10 mL). The combined organic layers were washed with brine (3 mL), dried over anhydrous Na₂SO₄, filtered, and concentrated under reduced pressure. The residue was purified by silica gel column chromatography. LCMS: m/z=331.0, 333.0, 335.0 [M+H]⁺.

2-(4-bromo-6-chloro-1-oxophthalazin-2(1H)-yl)acetic acid: To a solution of methyl 2-(4-bromo-6-chloro-1-oxophthalazin-2(1H)-yl)acetate (500 mg, 1.51 mmol) in THF (5 mL) and H₂O (5 mL) was added LiOH·H₂O (158 mg, 3.77 mmol). The mixture was stirred for 2 h. The mixture was diluted with water (3 mL) and washed with MTBE (2 mL). The aqueous phase was adjusted pH=2 with aq. HCl (3M) and extracted with EtOAc (3×2 mL). The combined organic layers were washed with brine (3 mL), dried over anhydrous Na₂SO₄, filtered, and concentrated under reduced pressure to provide a residue that was used directly.

2-(4-bromo-6-chloro-1-oxophthalazin-2(1H)-yl)-N-(5-fluoropyrimidin-2-yl)acetamide: To a solution of 2-(4-bromo-6-chloro-1-oxophthalazin-2(1H)-yl)acetic acid (90 mg, 0.28 mmol) and 5-fluoropyrimidin-2-amine (38 mg, 0.34 mmol) in pyridine (1.0 mL) was added EDCI (81 mg, 0.43 mmol). The mixture was stirred for 16 h. The mixture was diluted with H₂O (4 mL) and extracted with EtOAc (3×10 mL). The combined organic layers were washed with brine (3 mL), dried over anhydrous Na₂SO₄, filtered, and concentrated under reduced pressure to provide a residue that was used directly. LCMS: m/z=412.0, 414.0, 415.9 [M+H]⁺.

2-(6-chloro-4-cyclopropyloxy-1-oxophthalazin-2-yl)-N-(5-fluoropyrimidin-2-yl)acetamide: To a solution of cyclopropanol (14 mg, 0.24 mmol) in DMF (1.0 mL) at 0° C. was added NaH (4.8 mg, 0.12 mmol, 60% purity). The mixture was stirred at 0° C. for 0.5 h. To the mixture was added 2-(4-bromo-6-chloro-1-oxophthalazin-2(1H)-yl)-N-(5-fluoropyrimidin-2-yl)acetamide (50 mg, 0.12 mmol) at 0° C. and the mixture was stirred at 20° C. for 2 h. The reaction mixture was cooled to 0° C., diluted with aq. sat. NH₄Cl (10 mL), and extracted with EtOAc (4×5 mL). The combined organic layers were washed with brine (10 mL), dried over anhydrous Na₂SO₄, filtered, and concentrated under reduced pressure. The residue was purified by reverse-phase preparative HPLC. LCMS: m/z=390.2, 392.2 [M+H]⁺. ¹H NMR (400 MHz, CDCl₃): δ 8.92 (br s, 1H), 8.48 (s, 2H), 8.39 (d, J=8.8 Hz, 1H), 7.89 (d, J=2.0 Hz, 1H), 7.76 (dd, J=2.4, 8.8 Hz, 1H), 5.26 (br s, 2H), 4.30-4.25 (m, 1H), 0.86-0.84 (m, 4H).

### Example 35 and 36

### 2-[6-bromo-4-[cis-2-ethylcyclopropyl]oxy-1-oxophthalazin-2-yl]-N-(5-fluoropyrimidin-2-yl)acetamide

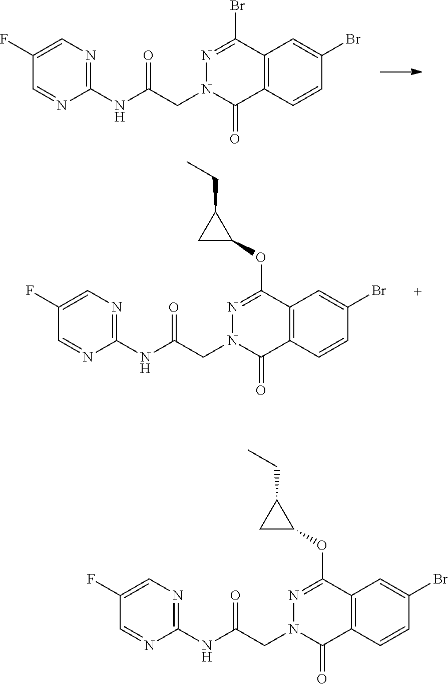

To a mixture of NaH (52 mg, 1.31 mmol, 60% purity) in DMA (3 mL) at 0° C. was added cis-2-ethylcyclopropanol (226 mg, 2.63 mmol). The mixture was stirred at 0° C. for 30 min. To the mixture was added 2-(4,6-dibromo-1-oxophthalazin-2(1H)-yl)-N-(5-fluoropyrimidin-2-yl)acetamide (200 mg, 0.44 mmol). The mixture was stirred at 0° C. for 2 h. The reaction mixture was diluted with H₂O (5 mL) and extracted with EtOAc (3×3 mL). The combined organic layers were washed with brine (3 mL), dried over anhydrous Na₂SO₄, filtered, and concentrated under reduced pressure. The residue was purified by reverse-phase preparative HPLC and further purified by chiral SFC (Column: Chiralpak IC-3 (50 mm×4.6 mm, 3 μm particle size); Mobile phase: A: CO₂ B: 0.1% i-PrNH₂ in i-PrOH; Gradient: B %=38% isocratic; Detection wavelength: 220 nm; Flow rate: 4 mL/min; Column temperature: 35° C.; System back pressure: 124 bar) to provide:

2-[6-bromo-4-[cis-2-ethylcyclopropyl]oxy-1-oxophthalazin-2-yl]-N-(5-fluoropyrimidin-2-yl)acetamide (first eluting isomer, 35): LCMS: m/z=462.1, 464.1 [M+H]⁺. ¹H NMR (400 MHz, CDCl₃) δ 9.19 (br s, 1H), 8.50 (s, 2H), 8.36-8.25 (m, 1H), 8.05 (d, J=1.6 Hz, 1H), 7.95-7.85 (m, 1H), 5.27 (br s, 2H), 4.30-4.26 (m, 1H), 1.60-1.49 (m, 1H), 1.47-1.30 (m, 1H), 1.09-0.95 (m, 5H), 0.55-0.48 (m, 1H)

2-[6-bromo-4-[cis-2-ethylcyclopropyl]oxy-1-oxophthalazin-2-yl]-N-(5-fluoropyrimidin-2-yl)acetamide (second eluting isomer, 36): LCMS: m/z=462.1, 464.1 [M+H]⁺. ¹H NMR (400 MHz, CDCl₃) δ 9.19 (br s, 1H), 8.50 (s, 2H), 8.36-8.25 (m, 1H), 8.05 (d, J=1.6 Hz, 1H), 7.95-7.85 (m, 1H), 5.27 (br s, 2H), 4.30-4.26 (m, 1H), 1.60-1.49 (m, 1H), 1.47-1.30 (m, 1H), 1.09-0.95 (m, 5H), 0.53-0.51 (m, 1H).

### Example 37

### 2-[4-cyclopropyloxy-6-(difluoromethoxy)-1-oxophthalazin-2-yl]-N-(5-fluoropyrimidin-2-yl)acetamide

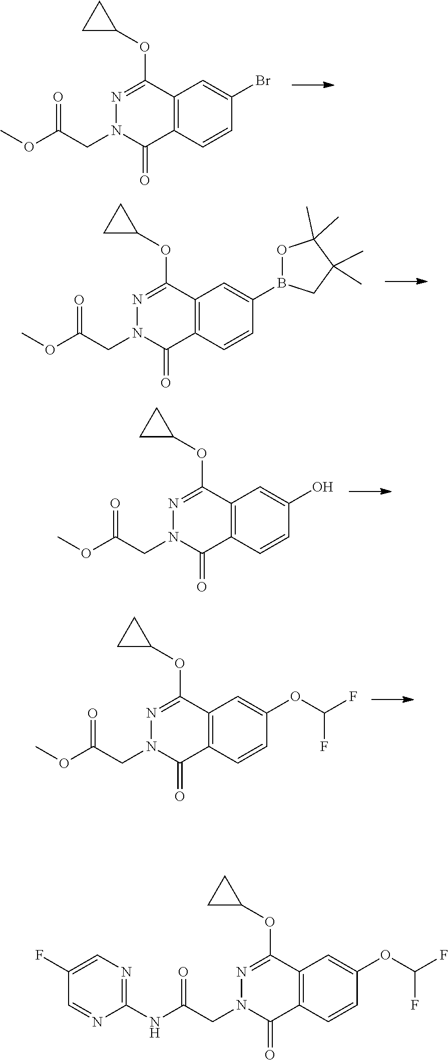

methyl 2-(4-cyclopropoxy-1-oxo-6-(4,4,5,5-tetramethyl-1,3,2-dioxaborolan-2-yl)phthalazin-2(1H)-yl)acetate: To a solution of methyl 2-(6-bromo-4-cyclopropoxy-1-oxophthalazin-2(1H)-yl)acetate (1.0 g, 2.83 mmol) and 4,4,4′,4′,5,5,5′,5′-octamethyl-2,2′-bi(1,3,2-dioxaborolane) (1.08 g, 4.25 mmol) in 1,4-dioxane (15 mL) were added KOAc (833 mg, 8.49 mmol) and Pd(dppf)Cl₂ (207 mg, 0.28 mmol). The mixture was stirred at 80° C. for 12 h. The mixture was diluted with H₂O (30 mL) and extracted with EtOAc (3×10 mL). The combined organic layers were washed with brine (15 mL), dried over anhydrous Na₂SO₄, filtered, and concentrated under reduced pressure. The residue was purified by silica gel column chromatography. LCMS: m/z=401.0 [M+H]⁺.

methyl 2-(4-cyclopropoxy-6-hydroxy-1-oxophthalazin-2(1H)-yl)acetate: To a solution of methyl 2-(4-cyclopropoxy-1-oxo-6-(4,4,5,5-tetramethyl-1,3,2-dioxaborolan-2-yl)phthalazin-2(1H)-yl)acetate (1.0 g, 2.50 mmol) in 1,4-dioxane (10 mL) and H₂O (10 mL) at 0° C. was added Oxone (1.69 g, 2.75 mmol). The mixture was stirred at 20° C. for 4 h. The mixture was diluted with H₂O (20 mL) and extracted with EtOAc (3×8 mL). The combined organic layers were washed with brine (15 mL), dried over anhydrous Na₂SO₄, filtered, and concentrated under reduced pressure. The residue was purified by silica gel column chromatography. LCMS: m/z=291.0 [M+H]⁺.

methyl 2-(4-cyclopropoxy-6-(difluoromethoxy)-1-oxophthalazin-2(1H)-yl)acetate: To a solution of methyl 2-(4-cyclopropoxy-6-hydroxy-1-oxophthalazin-2(1H)-yl)acetate (500 mg, 1.72 mmol) in DMF (8.0 mL) were added sodium 2-chloro-2,2-difluoroacetate (604 mg, 3.96 mmol) and K₂CO₃ (476 mg, 3.45 mmol). The mixture was stirred at 110° C. for 16 h. The mixture was diluted with H₂O (15 mL) and extracted with EtOAc (3×5 mL). The combined organic layers were washed with brine (15 mL), dried over anhydrous Na₂SO₄, filtered, and concentrated under reduced pressure. The residue was purified by silica gel column. LCMS: m/z=341.0 [M+H]⁺.

2-[4-cyclopropyloxy-6-(difluoromethoxy)-1-oxophthalazin-2-yl]-N-(5-fluoropyrimidin-2-yl)acetamide: To a solution of methyl 2-(4-cyclopropoxy-6-(difluoromethoxy)-1-oxophthalazin-2(1H)-yl)acetate (100 mg, 0.29 mmol) and 5-fluoropyrimidin-2-amine (83 mg, 0.74 mmol) in DCE (3 mL) was added AlMe₃ (1 M in heptane, 0.88 mL). The mixture was stirred at 60° C. for 12 h. The mixture was diluted with H₂O (10 mL) and extracted with EtOAc (3×3 mL). The combined organic layers was washed with brine (5 mL), dried over anhydrous Na₂SO₄, filtered and concentrated under reduced pressure. The residue was purified by reverse-phase preparative HPLC. LCMS: m/z=422.0 [M+H]⁺. ¹H NMR (400 MHz, DMSO-d₆): δ 11.07 (br s, 1H), 8.78 (s, 2H), 8.30 (d, J=8.8 Hz, 1H), 7.76-7.33 (m, 3H), 5.03 (s, 2H), 4.28-4.19 (m, 1H), 0.75-0.85 (m, 4H).

### Example 38

### 2-(6-bromo-4-cyclopropyloxy-1-oxophthalazin-2-yl)-N-(6-methyl-1,2,4-triazin-3-yl)acetamide

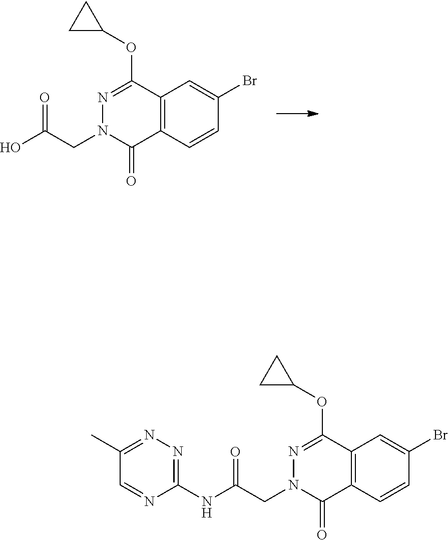 [SMILES: O=C(O)Cn1nc(OC2CC2)c2cc(Br)ccc2c1=O ; Cc1cnc(NC(=O)Cn2nc(OC3CC3)c3cc(Br)ccc3c2=O)nn1]

Solution A: To a solution of 2-(6-bromo-4-cyclopropoxy-1-oxophthalazin-2(1H)-yl)acetic acid (222 mg, 0.65 mmol) in THF (3.0 mL) was added CDI (176 mg, 1.09 mmol), stirred at for 1 h, and then cooled to 0° C. Solution B: To a solution of 6-methyl-1,2,4-triazin-3-amine (60 mg, 0.544 mmol) in THF (1.0 mL) at 0° C. was added LiHMDS (1 M in THF, 0.76 mmol) and then stirred at 0° C. for 0.5 h. Solution B was then added into Solution A at 0° C. and the combined mixture was stirred at 20° C. for 2 h. The reaction mixture was diluted with H₂O (10 mL) and extracted with EtOAc (3×10 mL). The combined organic layers were washed with brine (10 mL), dried over anhydrous Na₂SO₄, filtered, and concentrated under reduced pressure. The reside was purified by reverse-phase preparative HPLC. LCMS: m/z=431.0, 433.0 [M+H]⁺. ¹H NMR (400 MHz, CDCl₃): δ 8.94 (br, s, 1H), 8.40 (s, 1H), 8.29 (d, J=8.4 Hz, 1H), 8.07 (d, J=1.6 Hz, 1H), 7.90 (dd, J=1.6, 8.4 Hz, 1H), 5.31 (s, 2H), 4.38-4.12 (m, 1H), 2.70 (s, 3H), 0.90-0.79 (m, 4H).

### Example 39

### 2-(6-cyclobutyl-4-cyclopropyloxy-1-oxophthalazin-2-yl)-N-(5-fluoropyrimidin-2-yl)acetamide

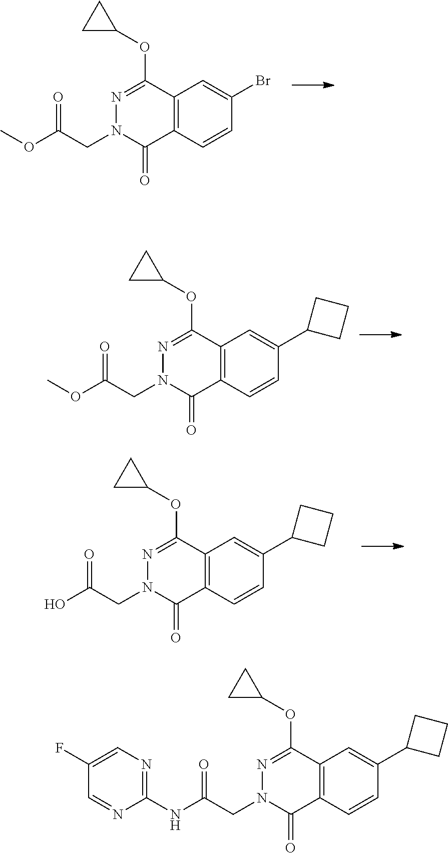 [SMILES: COC(=O)Cn1nc(OC2CC2)c2cc(Br)ccc2c1=O ; COC(=O)Cn1nc(OC2CC2)c2cc(C3CCC3)ccc2c1=O ; O=C(O)Cn1nc(OC2CC2)c2cc(C3CCC3)ccc2c1=O ; O=C(Cn1nc(OC2CC2)c2cc(C3CCC3)ccc2c1=O)Nc1ncc(F)cn1]

methyl 2-(6-cyclobutyl-4-cyclopropoxy-1-oxophthalazin-2(1H)-yl)acetate: A mixture of methyl 2-(6-bromo-4-cyclopropoxy-1-oxophthalazin-2(1H)-yl)acetate (400 mg, 1.13 mmol), potassium cyclobutyltrifluoroborate (275 mg, 1.70 mmol), Na₂CO₃ (240 mg, 2.27 mmol), 4-tert-butyl-2-(4-tert-butyl-2-pyridyl)pyridine (15 mg, 0.06 mmol), bis[3,5-difluoro-2-[5-(trifluoromethyl)-2-pyridyl]phenyl]iridium(1+); 2-(2-pyridyl)pyridine; hexafluorophosphate (11 mg, 0.011 mmol), and dichloronickel; 1,2-dimethoxyethane (12 mg, 0.056 mmol) in DMA (8.0 mL) was stirred under 36 W blue LED light for 12 h. The reaction mixture was poured into ice-cold H₂O (15 mL) and extracted with EtOAc (3×10 mL). The combined organic layers were washed with brine (2×10 mL), dried over anhydrous Na₂SO₄, filtered, and concentrated under reduced pressure. The residue was purified by silica gel column chromatography and further triturated with MTBE (5 mL). LCMS: m/z=329.1 [M+H]⁺.

2-(6-cyclobutyl-4-cyclopropoxy-1-oxophthalazin-2(1H)-yl)acetic acid: To a solution of methyl 2-(6-cyclobutyl-4-cyclopropoxy-1-oxophthalazin-2(1H)-yl)acetate (250 mg, 0.76 mmol) in THF (3.0 mL) and H₂O (3.0 mL) was added LiOH·H₂O (80 mg, 1.90 mmol). The mixture was stirred for 1 h. The reaction mixture was diluted with H₂O (20 mL) and washed with MTBE (10 mL). The aqueous layer was acidified with aq. HCl (3M) to pH=3 and extracted with EtOAc (3×15 mL). The combined organic layers were washed with brine (20 mL), dried over anhydrous Na₂SO₄, filtered, and concentrated under reduced pressure to provide a residue that was used directly. LCMS: m/z=315.2 [M+H]⁺.

2-(6-cyclobutyl-4-cyclopropyloxy-1-oxophthalazin-2-yl)-N-(5-fluoropyrimidin-2-yl)acetamide: To a mixture of 2-(6-cyclobutyl-4-cyclopropoxy-1-oxophthalazin-2(1H)-yl)acetic acid (60 mg, 0.19 mmol) and 5-fluoropyrimidin-2-amine (65 mg, 0.5 mmol) in pyridine (1.5 mL) was added EDCI (110 mg, 0.57 mmol). The mixture was stirred for 12 h. The reaction mixture was diluted with H₂O (5 mL) and extracted with EtOAc (3×10 mL). The combined organic layers were washed with brine (15 mL), dried over anhydrous Na₂SO₄, filtered, and concentrated under reduced pressure. The residue was purified by reverse-phase preparative HPLC. LCMS: m/z=410.1 [M+H]⁺. ¹H NMR (400 MHz, CDCl₃): δ 9.22 (br s, 1H), 8.48 (br s, 2H), 8.37 (d, J=8.4 Hz, 1H), 7.72-7.61 (m, 2H), 5.20 (br s, 2H), 4.29-4.25 (m, 1H), 3.74-3.67 (m, 1H), 2.51-2.38 (m, 2H), 2.28-2.04 (m, 3H), 1.98-1.86 (m, 1H), 0.90-0.80 (m, 4H).

The following compounds as shown in Table 1 were, or can be, made via similar procedures as those described above using the appropriate starting materials.

| Ex. | Name | NMR | LCMS |
|---|---|---|---|
| 40 | 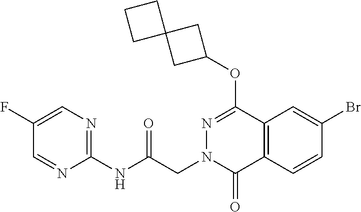 [SMILES: O=C(Cn1nc(OC2CC3(CCC3)C2)c2cc(Br)ccc2c1=O)Nc1ncc(F)cn1] 2-(6-bromo-1-oxo-4-spiro[3.3]heptan-2- yloxyphthalazin-2-yl)-N-(5-fluoropyrimidin-2- yl)acetamide | ¹H NMR (400 MHz, DMSO- d₆): δ 11.09 (s, 1H), 8.78 (d, J = 0.7 Hz, 2H), 8.16-8.14 (m, 1H), 8.10-8.08 (m, 2H), 4.98 (s, 2H), 4.92 (quintet, J = 7.1 Hz, 1H), 2.57-2.53 (m, 2H), 2.13 (ddd, J = 9.9, 7.2, 2.8 Hz, 2H), 2.03-1.96 (m, 4H), 1.84-1.76 (m, 2H) | m/z = 488.4, 490.6 [M + H]⁺ |
| 41 | 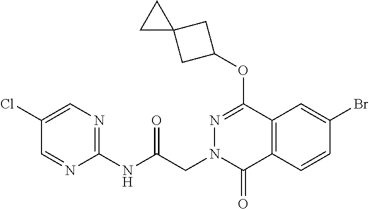 [SMILES: O=C(Cn1nc(OC2CC3(CC3)C2)c2cc(Br)ccc2c1=O)Nc1ncc(Cl)cn1] 2-(6-bromo-1-oxo-4-spiro[2.3]hexan-5- yloxyphthalazin-2-yl)-N-(5-chloropyrimidin-2- yl)acetamide | ¹H NMR (400 MHz, DMSO- d₆): δ 11.10 (s, 1H), 8.79 (d, J = 0.7 Hz, 2H), 8.17-8.09 (m, 3H), 5.00-4.88 (m, 3H), 4.58 (d, J = 20.5 Hz, 4H), 2.79-2.74 (m, 2H), 2.41-2.36 (m, 2H) | m/z = 490.2, 492.2, 494.2 [M + H]⁺ |
| 42 | 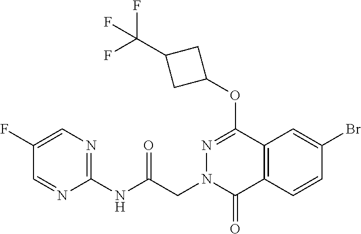 [SMILES: O=C(Cn1nc(OC2CC(C(F)(F)F)C2)c2cc(Br)ccc2c1=O)Nc1ncc(F)cn1] 2-[6-bromo-1-oxo-4-[3- (trifluoromethyl)cyclobutyl]oxyphthalazin-2- yl]-N-(5-fluoropyrimidin-2-yl)acetamide | ¹H NMR (400 MHz, DMSO- d₆): δ 11.09 (d, J = 1.4 Hz, 1H), 8.78 (dd, J = 1.4, 0.7 Hz, 2H), 8.21-8.10 (m, 3H), 5.17- 4.98 (m, 3H), 2.74-2.67 (m, 1H), 2.61-2.57 (m, 3H), 2.31- 2.23 (m, 1H) | m/z = 516.3, 519.3 [M + H]⁺ |
| 43 | 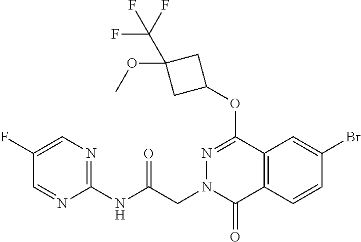 [SMILES: COC1(C(F)(F)F)CC(Oc2nn(CC(=O)Nc3ncc(F)cn3)c(=O)c3ccc(Br)cc23)C1] 2-[6-bromo-1-oxo-4-[3- (trifluoromethyl)cyclobutyl]oxyphthalazin-2- yl]-N-(5-fluoropyrimidin-2-yl)acetamide (single unknown stereoisomer) | ¹H NMR (400 MHz, DMSO- d₆): δ 11.09 (s, 1H), 8.78 (s, 2H), 8.23 (dd, J = 1.9, 0.6 Hz, 1H), 8.17 (dd, J = 8.5, 0.6 Hz, 1H), 8.12 (dd, J = 8.5, 1.9 Hz, 1H), 5.01 (s, 2H), 4.87-4.80 (m, 1H), 3.37 (s, 3H), 2.93- 2.87 (m, 2H), 2.71-2.65 (m, 2H) | m/z = 546.3, 548.3 [M + H]⁺ |
| 44 | 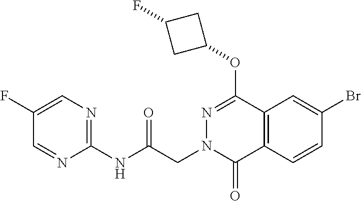 2-[6-bromo-4-(cis-3-fluorocyclobutyl)oxy-1- oxophthalazin-2-yl]-N-(5-fluoropyrimidin-2- yl)acetamide | ¹H NMR (400 MHz, DMSO- d₆): δ 11.10 (s, 1H), 8.78 (d, J = 0.7 Hz, 2H), 8.19-8.15 (m, 2H), 8.12 (dd, J = 8.5, 1.9 Hz, 1H), 5.00-4.81 (m, 3H), 4.70-4.64 (m, 1H), 2.46-2.33 (m, 4H) | m/z = 466.2, 468.2 [M + H]⁺ |
| 45 | 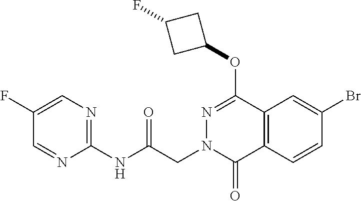 2-[6-bromo-4-(trans-3-fluorocyclobutyl)oxy-1- oxophthalazin-2-yl]-N-(5-fluoropyrimidin-2- yl)acetamide | ¹H NMR (400 MHz, CDCl₃): δ 9.14 (s, 1H), 8.51 (s, 2H), 8.30 (dd, J = 8.5, 0.5 Hz, 1H), 8.22-8.13 (m, 1H), 7.92 (dd, J = 8.5, 1.9 Hz, 1H), 5.50-5.15 (m, 4H), 2.98-2.59 (m, 4H) | m/z = 466.1, 468.1 [M + H]⁺ |
| 46 | 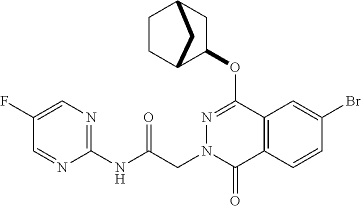 2-[4-[[(1r,2r,4s)-2-bicyclo[2.2.1]heptanyl]oxy]- 6-bromo-1-oxophthalazin-2-y1]-N-(5- fluoropyrimidin-2-yl)acetamide | ¹H NMR (400 MHz, DMSO- d₆): δ 11.09 (s, 1H), 8.79 (s, 2H), 8.16-8.08 (m, 3H), 5.04- 4.94 (m, 2H), 4.68-4.66 (m, 1H), 2.30-2.29 (m, 1H), 1.81- 1.76 (m, 1H), 1.68-1.39 (m, 4H), 1.24-1.07 (m, 4H) | m/z = 488.3, 490.3 [M + H]⁺ |
| 47 | 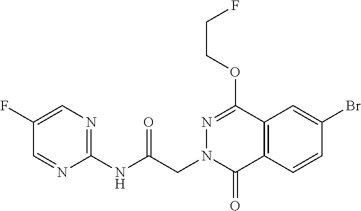 [SMILES: O=C(Cn1nc(OCCF)c2cc(Br)ccc2c1=O)Nc1ncc(F)cn1] 2-[6-bromo-4-(2-fluoroethoxy)-1- oxophthalazin-2-yl]-N-(5-fluoropyrimidin-2- yl)acetamide | ¹H NMR (400 MHz, DMSO- d₆): δ 11.12 (s, 1H), 8.78 (s, 2H), 8.19-8.12 (m, 3H), 5.01 (s, 2H), 4.91-4.77 (m, 2H), 4.55-4.46 (m, 2H) | m/z = 440.2, 442.2 [M + H]⁺ |
| 48 | 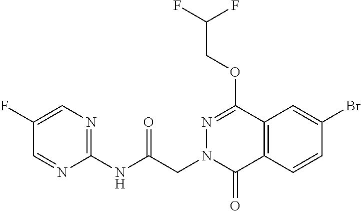 [SMILES: O=C(Cn1nc(OCC(F)F)c2cc(Br)ccc2c1=O)Nc1ncc(F)cn1] 2-[6-bromo-4-(2,2-difluoroethoxy)-1- oxophthalazin-2-yl]-N-(5-fluoropyrimidin-2- yl)acetamide | ¹H NMR (400 MHz, DMSO- d₆): δ 11.11 (br s, 1H), 8.77 (s, 2H), 8.26-8.02 (m, 3H), 6.68- 6.30 (m, 1H), 5.17-4.95 (m, 2H), 4.62-4.46 (m, 2H) | m/z = 457.9, 459.9 [M + H]⁺ |
| 49 | 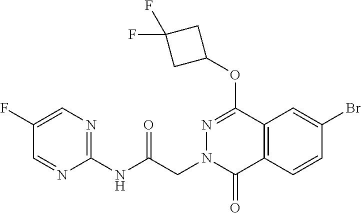 [SMILES: O=C(Cn1nc(OC2CC(F)(F)C2)c2cc(Br)ccc2c1=O)Nc1ncc(F)cn1] 2-[6-bromo-4-(3,3-difluorocyclobutyl)oxy-1- oxophthalazin-2-yl]-N-(5-fluoropyrimidin-2- yl)acetamide | ¹H NMR (400 MHz, CDCl₃): δ 9.01 (br s, 1H), 8.50 (s, 2H), 8.30 (d, J = 8.4 Hz, 1H), 8.13 (d, J = 1.8 Hz, 1H), 7.93 (dd, J = 1.8, 8.4 Hz, 1H), 5.27 (s, 2H), 5.14-5.02 (m, 1H), 3.24- 3.05 (m, 2H), 2.96-2.73 (m, 2H | m/z = 483.9, 485.9 [M + H]⁺ |
| 50 | 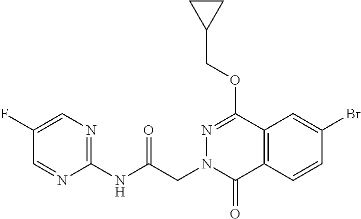 [SMILES: O=C(Cn1nc(OCC2CC2)c2cc(Br)ccc2c1=O)Nc1ncc(F)cn1] 2-[6-bromo-4-(cyclopropylmethoxy)-1- oxophthalazin-2-yl]-N-(5-fluoropyrimidin-2- yl)acetamide | 1H NMR (400 MHz, DMSO- d₆): δ 11.08 (br s, 1H), 8.77 (s, 2H), 8.20-8.04 (m, 3H), 4.99 (s, 2H), 4.10 (d, J = 8 Hz, 2H), 1.41-1.27 (m, 1H), 0.67- 0.51 (m, 2H), 0.47-0.31 (m, 2H) | m/z = 448.1, 450.1 [M + H]⁺ |
| 51 | 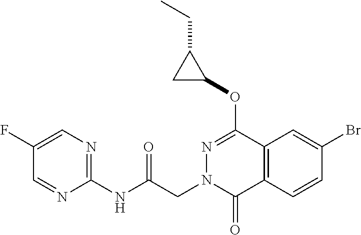 2-[6-bromo-4-[trans-2-ethylcyclopropyl]oxy-1- oxophthalazin-2-yl]-N-(5-fluoropyrimidin-2- yl)acetamide | ¹H NMR (400 MHz, CDCl₃) δ 9.01 (br s, 1H), 8.49 (s, 2H), 8.29 (d, J = 8.8 Hz, 1H), 8.06 (d, J = 1.6 Hz, 1H), 7.89 (dd, J = 1.6, 8.8 Hz, 1H), 5.26 (br s, 2H), 4.00-3.88 (m, 1H), 1.40-1.30 (m, 2H), 1.20-1.09 (m, 1H), 1.03 (t, J = 7.2 Hz, 3H), 1.00-0.94 (m, 1H), 0.65 (q, J = 6.4 Hz, 1H) | m/z = 462.1, 464.1 [M + H]⁺ |
| 52 | 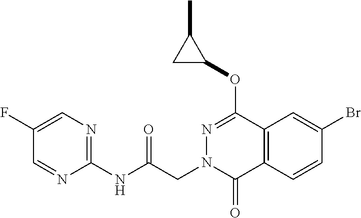 2-[6-bromo-4-[cis-2-methylcyclopropyl]oxy-1- oxophthalazin-2-yl]-N-(5-fluoropyrimidin-2- yl)acetamide | ¹H NMR (400 MHz, DMSO- d₆): δ 11.10 (br s, 1H), 8.78 (s, 2H), 8.26-7.99 (m, 3H), 5.18- 4.86 (m, 2H), 4.21-4.17 (m, 1H), 1.11-1.02 (m, 4H), 0.96- 0.94 (m, 1H), 0.59-0.48 (m, 1H) | m/z = 447.9, 449.9 [M + H]⁺ |

### Example 53

### 2-[6-bromo-4-[2-trans-fluorocyclopropyl]oxy-1-oxophthalazin-2-yl]-N-(5-fluoropyrimidin-2-yl)acetamide

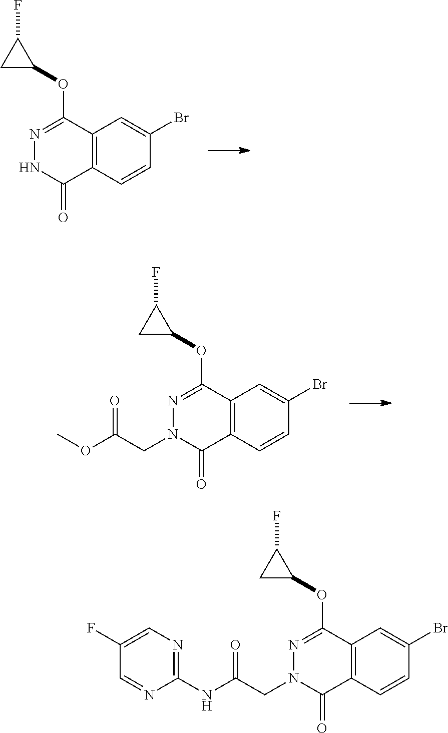

methyl 2-(6-bromo-4-(2-trans-fluorocyclopropoxy)-1-oxophthalazin-2(1H)-yl)acetate: To a solution of 6-bromo-4-(2-trans-fluorocyclopropoxy)phthalazin-1(2H)-one (270 mg, 0.91 mmol) in DMF (2 mL) was added Cs₂CO₃ (588 mg, 1.81 mmol) and methyl 2-iodoacetate (271 mg, 1.35 mmol). The mixture was stirred for 2 h. The mixture was diluted with H₂O (5 mL) and extracted with EtOAc (3×5 mL). The combined organic layers were washed with brine (5 mL), dried over anhydrous Na₂SO₄, filtered and concentrated under reduced pressure. The residue was purified by silica gel column chromatography. LCMS: m/z=371.0, 373.0 [M+H]⁺.

2-(6-bromo-4-(2-trans-fluorocyclopropoxy)-1-oxophthalazin-2(1H)-yl)-N-(5-fluoropyrimidin-2-yl)acetamide: To a solution of 5-fluoropyrimidin-2-amine (12 mg, 0.107 mmol) in DCE (1 mL) was added AlMe₃ (1 M in n-hexane, 0.11 mL). The mixture was stirred for 0.5 h. Methyl 2-(6-bromo-4-(2-trans-fluorocyclopropoxy)-1-oxophthalazin-2(1H)-yl)acetate (20 mg, 0.05 mmol) in DCE (1 mL) was added and the mixture was stirred at 60° C. for 12 h. The mixture was poured into H₂O (5 mL) and extracted with EtOAc (3×5 mL). The combined organic layers were dried over anhydrous Na₂SO₄, filtered and concentrated under reduced pressure. The residue was purified by reverse-phase preparative HPLC. LCMS: m/z=452.0, 454.0 [M+H]⁺. ¹H NMR (400 MHz, DMSO-d₆): δ 8.82 (br s, 1H), 8.49 (s, 2H), 8.29 (d, J=8.4 Hz, 1H), 8.01 (d, J=1.6 Hz, 1H), 7.91 (d, J=8.4, 1.6 Hz, 1H), 5.39-5.32 (m, 2H), 4.89-4.72 (m, 1H), 4.55-4.50 (m, 1H), 1.57-1.49 (m, 1H), 1.31-1.26 (m, 1H).

### Example 54

### 2-[6-bromo-4-[2-cis-fluorocyclopropyl]oxy-1-oxophthalazin-2-yl]-N-(5-fluoropyrimidin-2-yl)acetamide

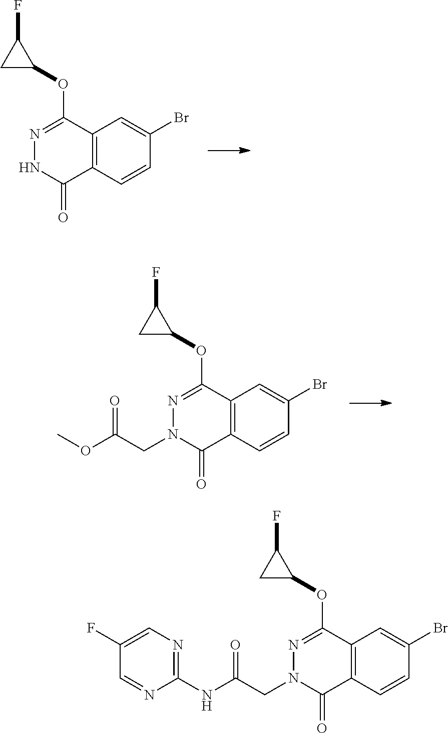

methyl 2-(6-bromo-4-(2-cis-fluorocyclopropoxy)-1-oxophthalazin-2(1H)-yl)acetate: To a solution of 6-bromo-4-(2-cis-fluorocyclopropoxy)phthalazin-1(2H)-one (210 mg, 0.702 mmol) in DMF (2 mL) were added Cs₂CO₃ (457 mg, 1.40 mmol) and methyl 2-iodoacetate (210 mg, 1.05 mmol). The mixture was stirred for 2 h. The mixture was diluted with H₂O (5 mL) and extracted with EtOAc (3×5 mL). The combined organic layers were washed with brine (5 mL), dried over anhydrous Na₂SO₄, filtered and concentrated under reduced pressure. The residue was purified by silica gel column chromatography. LCMS: m/z=370.9, 373.0 [M+H]⁺.

2-[6-bromo-4-[2-cis-fluorocyclopropyl]oxy-1-oxophthalazin-2-yl]-N-(5-fluoropyrimidin-2-yl)acetamide: To a solution of 5-fluoropyrimidin-2-amine (49 mg, 0.43 mmol) in DCE (1 mL) was added AlMe₃ (1 M in n-hexane, 0.43 mL). The mixture was stirred for 0.5 h. methyl 2-(6-bromo-4-(2-cis-fluorocyclopropoxy)-1-oxophthalazin-2(1H)-yl)acetate (80 mg, 0.21 mmol) in DCE (1 mL) was added and the mixture was stirred at 60° C. for 12 h. The mixture was poured into H₂O (5 mL) and extracted with EtOAc (3×5 mL). The organic layers were dried over anhydrous Na₂SO₄, filtered and concentrated under reduced pressure. The residue was purified by reverse-phase preparative HPLC. LCMS: m/z=451.9, 453.8 [M+H]⁺. ¹H NMR (400 MHz, DMSO-d₆): δ 11.1 (br s, 1H), 8.78 (s, 2H), 8.18-8.08 (m, 3H), 5.08-4.75 (m, 3H), 4.15-4.11 (m, 1H), 1.41-1.23 (m, 2H).

### Biological Example 1

### Biochemical Assay of the Compounds

### Procedure for Culturing THP-1 Cells

Compounds as provided herein were tested in the following assay. Cell culture medium employed contained RPMI 1640 medium (89%), FBS (10%), Pen/Strep (1%), and 2-mercaptoethanol (0.05 mM). Freezing medium was made up of 90% FBS and 10% DMSO. THP-1 cells were removed from the liquid nitrogen and placed into a 37° C. water bath to thaw, until signs of ice dissipated. The cells were then added to 9 mL of warm cell culture medium and centrifuged for 5 minutes at 1000 rpm. The supernatant was discarded, and the cells were resuspended in new cell culture medium. THP-1 cells were then split and cultured in the cell culture medium, being passaged every 2-3 days with the cell density will be maintained between 5×10⁵ and 1.5×10⁶ viable cells/mL.

To freeze, cells were resuspended with fresh freezing medium, adjusting the cell density to 5×10⁶ cells/mL. The cell suspension was partitioned into 1 mL aliquots per vial, and the vials were transferred to a −80° C. freezer. After one day at −80° C., the cell vials were transferred to liquid nitrogen freezer for storage.

### Procedure for IL-1β Secretion Assay in 384-Well Plates

PMA was dissolved in DMSO to make a stock solution at 5 mg/ml and stored in 10 μl aliquots at −20° C. for single use. PMA is added to normal growth medium. LPS was diluted with 1 mL of water solution to provide a 1 mg/mL stock solution and stored in 15 μl aliquots at −20° C. for single use. Nigericin is diluted in ice cold 100% ethanol to 5 mg/ml (6.7 mM) and stored in 75 μL aliquots at −20° C. for single use. Serum-free media contains RPMI 1640 medium (99%), Pen/Strep (1%), and 2-mercaptoethanol (0.05 mM). The two control conditions used to qualify and normalize test compound dose-response curves were as follows: High Control=25 ng/mL LPS, 5 M Nigericin, 0.5% DMSO, Low Control=25 ng/mL, LPS, 0.5% DMSO.

Day 1: Differentiation with PMA

THP-1 cells were diluted to provide a suspension at a concentration of 1.0×10⁶ cells/mL with the total volume of suspension required to enable the desired number of assay plates. The growth media was supplemented with PMA (5 ng/mL final concentration) and the cells were incubated at 37° C. under a humidified atmosphere of 5% CO₂ for 40 h.

Day 3: Plating with Sequential LPS and Nigericin Stimulation

All media was carefully aspirated from each culture flask. The cells were washed carefully with 1×DPBS. The cells were then briefly digested with trypsin LE for 5 minutes at 23° C. and immediately resuspended in cell growth media. After resuspension, the cells were centrifuged at 1000 rpm for 3 minutes and the supernatant was discarded. The cells were resuspended in DPBS and once again centrifuged at 1000 rpm for 5 minutes. The supernatant was discarded and the cell pellet was resuspended in serum-free media supplemented with LPS (25 ng/mL final concentration) to enable the distribution of 30K THP-1 cells within 45 μL of media into each well of 384-well PDL-coated plates. The 384-well plates were then incubated at 37° C. under a humidified atmosphere of 5% CO₂ for 2 h. Following this period, test compounds were dispensed by Tecan across the desired concentration range with all wells normalized to a final 0.5% DMSO concentration. The plates were then then incubated at 37° C. under a humidified atmosphere of 5% CO₂ for 1 h. Following this period, 5 μL of the 5 mg/mL nigericin stock solution was added to each of the appropriate wells and plates were centrifuged at 1000 rpm for 30 seconds. The plates were the immediately reintroduced to the incubator at 37° C. under a humidified atmosphere of 5% CO₂ for 2 h. After this time, 35 μL/well of supernatant was collected and transferred into v-bottom plate and centrifuged at 1000 rpm for 1 minute. These supernatant aliquots were analyzed using an IL-Iβ detection kit as described below. If needed, the test samples could be snap frozen and stored at −80° C. until analyzed.

### IL-1β Detection

To prepare each ELISA plate, capture antibody (mAb Mt175) was diluted with PBS to a final concentration of 2 g/mL and then 20 L of this solution was added to each well of the ELISA plate.

Each plate was allowed to incubate overnight at 4° C. The next day, the capture antibody solution was removed and discarded. Each ELISA plate was washed 4 times with PBST followed by the addition of 25 μL/well of blocking buffer (Licor-927-40010) supplemented with 0.1% Tween 20. Each ELISA plate was then allowed to incubate for 1 hour at 23° C. After this time, the blocking buffer was removed and discarded. Each ELISA plate was washed 4 times with PBST. During this time, the v-bottomed plates containing the supernatant aliquots from the assay run were centrifuged at 300 g for 5 minutes before transferring 15 L/well of the supernatant sample to each ELISA plate. Each ELISA plate was then allowed to incubate for 2 h at 23° C. After this time, the supernatant samples were removed and discarded. Each ELISA plate was washed 4 times with PBST. To each ELISA plate was added 15 L/well of mAb7PO-biotin at 0.5 g/mL (1:1000 diluted in blocking buffer). Each ELISA plate was then allowed to incubate for 1 h at 23° C. After this time, the antibody solution was removed and discarded. Each ELISA plate was washed 4 times with PBST. To each ELISA plate was added 20 L/well of streptavidin-HRP (1:2000 diluted in blocking buffer). Each ELISA plate was then allowed to incubate for 1 h at 23° C. After this time, the buffer was removed and discarded. Each ELISA plate was washed 4 times with PBST. To each ELISA plate was added 20 L/well of HRP substrate. Each ELISA plate was then allowed to incubate for 2 minutes at 23° C. After this time, to each ELISA plate was added L/well of stop solution. Each ELISA plate was centrifuged at 1200 rpm for 30 seconds.

The plate was then read at 450 nm in a microplate reader. Percent inhibition was calculated as follows:

% inhibition rate=(treated samples-high control)/(low control-high control)×100

Activity of the tested compounds is provided in Table 3 and Table 4. In Table 3, activity is as follows: +++=IC₅₀<1 μM; ++=IC₅₀ 1-10 μM; +=IC₅₀>10 μM. In Table 4, the activity is expressed in μM.

TABLE 3

| Ex. | Activity |
|---|---|
| 1 | +++ |
| 2 | +++ |
| 3 | +++ |
| 4 | +++ |
| 5 | +++ |
| 6 | +++ |
| 7 | +++ |
| 8 | +++ |
| 9 | +++ |
| 10 | +++ |
| 11 | +++ |
| 12 | +++ |
| 13 | +++ |
| 14 | +++ |
| 15 | +++ |
| 16 | +++ |
| 17 | +++ |
| 18 | ++ |
| 19 | +++ |
| 20 | ++ |
| 21 | +++ |
| 22 | +++ |
| 23 | +++ |
| 24 | +++ |
| 25 | +++ |
| 26 | +++ |
| 27 | +++ |
| 28 | +++ |
| 29 | +++ |
| 30 | +++ |
| 31 | +++ |
| 32 | +++ |
| 33 | +++ |
| 34 | +++ |
| 35 | +++ |
| 36 | +++ |
| 37 | +++ |
| 38 | +++ |
| 39 | ++ |
| 40 | +++ |
| 41 | +++ |
| 42 | +++ |
| 43 | +++ |
| 44 | +++ |
| 45 | +++ |
| 46 | +++ |
| 47 | ++ |
| 48 | +++ |
| 49 | ++ |
| 50 | +++ |
| 51 | +++ |
| 52 | +++ |

TABLE 4

| Ex. | Activity |
|---|---|
| 1 | 0.010 |
| 2 | 0.202 |
| 3 | 0.035 |
| 4 | 0.033 |
| 5 | 0.021 |
| 6 | 0.308 |
| 7 | 0.084 |
| 8 | 0.728 |
| 9 | 0.554 |
| 10 | 0.141 |
| 11 | 0.136 |
| 12 | 0.152 |
| 13 | 0.034 |
| 14 | 0.314 |
| 15 | 0.019 |
| 16 | 0.137 |
| 17 | 0.070 |
| 18 | 1.51 |
| 19 | 0.179 |
| 20 | 3.40 |
| 21 | 0.031 |
| 22 | 0.815 |
| 23 | 0.312 |
| 24 | 0.183 |
| 25 | 0.033 |
| 26 | 0.596 |
| 27 | 0.235 |
| 28 | 0.021 |
| 29 | 0.026 |
| 30 | 0.050 |
| 31 | 0.066 |
| 32 | 0.183 |
| 33 | 0.625 |
| 34 | 0.408 |
| 35 | 0.368 |
| 36 | 0.397 |
| 37 | 0.424 |
| 38 | 0.444 |
| 39 | 1.12 |
| 40 | 0.017 |
| 41 | 0.052 |
| 42 | 0.36 |
| 43 | 0.447 |
| 44 | 0.492 |
| 45 | 0.912 |
| 46 | 0.862 |
| 47 | 1.40 |
| 48 | 0.568 |
| 49 | 1.51 |
| 50 | 0.343 |
| 51 | 0.790 |
| 52 | 0.924 |
| 53 | 0.389 |
| 54 | 0.49 |

Unless otherwise defined, all technical and scientific terms used herein have the same meaning as commonly understood by one of ordinary skill in the art to which this disclosure belongs.

The embodiments illustratively described herein may suitably be practiced in the absence of any element or elements, limitation or limitations, not specifically disclosed herein. Thus, for example, the terms “comprising,” “including,” “containing”, etc. shall be read expansively and without limitation.

Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the embodiments claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A compound or a pharmaceutically acceptable salt, isotopically enriched analog, stereoisomer, mixture of stereoisomers, or prodrug thereof, selected from Table 1.

2. A compound or a pharmaceutically acceptable salt, isotopically enriched analog, stereoisomer, mixture of stereoisomers, or prodrug thereof, selected from Table 2.

3. A pharmaceutical composition comprising a compound of claim 1 or 2, or a pharmaceutically acceptable salt, stereoisomer, mixture of stereoisomers, or prodrug thereof, and a pharmaceutically acceptable carrier.

4. A method for treating a disease or condition mediated, at least in part, by NLRP3, the method comprising administering an effective amount of the pharmaceutical composition of claim 3, to a subject in need thereof.

5. The method of claim 4, wherein the disease or condition is Alzheimer disease, atherosclerosis, asthma, allergic airway inflammation, cryopyrin-associated periodic syndromes, gout, inflammatory bowel disease and related disorders, nonalcoholic fatty liver disease (NAFLD), nonalcoholic steatohepatitis (NASH), hypertension, myocardial infarction, multiple sclerosis, experimental autoimmune encephalitis, oxalate-induced nephropathy, hyperinflammation following influenza infection, graft-versus-host disease, stroke, silicosis, type 1 diabetes, obesity-induced inflammation or insulin resistance, rheumatoid arthritis, myelodysplastic syndrome, contact hypersensitivity, joint inflammation triggered by chikungunya virus, or traumatic brain injury.

6. The method of claim 4, wherein the disease is nonalcoholic fatty liver disease (NAFLD) or nonalcoholic steatohepatitis (NASH).

7. The method of claim 4, wherein the disease is Alzheimer's disease.

8. Use of a compound of claim 1 or 2, or a pharmaceutically acceptable salt, stereoisomer, mixture of stereoisomers, or prodrug thereof, for treating a disease or condition mediated, at least in part, by NLRP3.

9. The use of claim 8, wherein the disease or condition is Alzheimer disease, atherosclerosis, asthma, allergic airway inflammation, cryopyrin-associated periodic syndromes, gout, inflammatory bowel disease and related disorders, nonalcoholic fatty liver disease (NAFLD), nonalcoholic steatohepatitis (NASH), hypertension, myocardial infarction, multiple sclerosis, experimental autoimmune encephalitis, oxalate-induced nephropathy, hyperinflammation following influenza infection, graft-versus-host disease, stroke, silicosis, type 1 diabetes, obesity-induced inflammation or insulin resistance, rheumatoid arthritis, myelodysplastic syndrome, contact hypersensitivity, joint inflammation triggered by chikungunya virus, or traumatic brain injury.

10. A compound of claim 1 or 2, or a pharmaceutically acceptable salt, stereoisomer, mixture of stereoisomers, or prodrug thereof, for use in therapy.

11. A compound of claim 1 or 2, or a pharmaceutically acceptable salt, stereoisomer, mixture of stereoisomers, or prodrug thereof, for use in treating Alzheimer's disease.

12. A compound of claim 1 or 2, or a pharmaceutically acceptable salt, stereoisomer, mixture of stereoisomers, or prodrug thereof, for use in treating nonalcoholic fatty liver disease (NAFLD) or nonalcoholic steatohepatitis (NASH).

13. The use of a compound of claim 1 or 2, or a pharmaceutically acceptable salt, stereoisomer, mixture of stereoisomers, or prodrug thereof, for the manufacture of a medicament for treating a neurodegenerative disease, treating Alzheimer's disease, atherosclerosis, asthma, allergic airway inflammation, cryopyrin-associated periodic syndromes, gout, inflammatory bowel disease and related disorders, nonalcoholic fatty liver disease (NAFLD), nonalcoholic steatohepatitis (NASH), hypertension, myocardial infarction, multiple sclerosis, experimental autoimmune encephalitis, oxalate-induced nephropathy, hyperinflammation following influenza infection, graft-versus-host disease, stroke, silicosis, type 1 diabetes, obesity-induced inflammation or insulin resistance, rheumatoid arthritis, myelodysplastic syndrome, contact hypersensitivity, joint inflammation triggered by chikungunya virus, or traumatic brain injury.
